SEQUENCE LISTING (12 sequences)

SEQ ID NO: 1 - skipped

SEQ ID NO: 2 - DNA, 65 bp, Artificial Sequence
  source: /note=“Description of Artificial Sequence: Synthetic oligonucleotide”
ttgccccacagggcagtaacggcagacttctcctcaggagtcaggtgcaccatggtgtctgtttg

SEQ ID NO: 3 - skipped

SEQ ID NO: 4 - DNA, 60 bp, Artificial Sequence
  source: /note=“Description of Artificial Sequence: Synthetic oligonucleotide”
aaagaataacagtgataatttctgggttaaggcaatagcaatatctctgcatataaatat

SEQ ID NO: 5 - DNA, 22 bp, Artificial Sequence
  source: /note=“Description of Artificial Sequence: Synthetic primer”
caccaacttcatccacgttcac

SEQ ID NO: 6 - DNA, 26 bp, Artificial Sequence
  source: /note=“Description of Artificial Sequence: Synthetic primer”
tctattgcttacatttgcttctgaca

SEQ ID NO: 7 - DNA, 22 bp, Artificial Sequence
  source: /note=“Description of Artificial Sequence: Synthetic oligonucleotide”
cttctccacaggagtcaggtgc

SEQ ID NO: 8 - DNA, 20 bp, Artificial Sequence
  source: /note=“Description of Artificial Sequence: Synthetic primer”
ttgtaaccttgataccaacc

SEQ ID NO: 9 - DNA, 20 bp, Artificial Sequence
  source: /note=“Description of Artificial Sequence: Synthetic primer”
cttacatttgcttctgacac

SEQ ID NO: 10 - DNA, 17 bp, Artificial Sequence
  source: /note=“Description of Artificial Sequence: Synthetic probe”
cagacttctccacagga

SEQ ID NO: 11 - DNA, 17 bp, Artificial Sequence
  source: /note=“Description of Artificial Sequence: Synthetic probe”
cagacttctcctcagga

SEQ ID NO: 12 - DNA, 18 bp, Artificial Sequence
  source: /note=“Description of Artificial Sequence: Synthetic oligonucleotide”
ctcgaggaagggacagta

(19) United States
(12) Patent Application Publication
(10) Pub. No.: US 2019/0298857 A1
(43) Pub. Date: Oct. 3, 2019

(54) POLYMER-BASED NANOPARTICLES, RELATED FORMULATIONS METHODS, AND APPARATUS

(71) Applicant: TRUCODE GENE REPAIR, INC., South San Francisco, CA (US)

(72) Inventors: Bo Ying, Arlington, MA (US); James M. Coull, Westford, MA (US); Derek Sim, Burlingame, CA (US)

(21) Appl. No.: 16/243,928

(22) Filed: Jan. 9, 2019

Related U.S. Application Data
(60) Provisional application No. 62/696,791, filed on Jul. 11, 2018.
(60) Provisional application No. 62/615,389, filed on Jan. 9, 2018.

(51) Int. Cl. A61K 47/69; C12N 15/113; B01J 14/00

(52) U.S. Cl. CPC A61K 47/6937; C12N 15/113; C12N 2310/351; C12N 2310/15; C12N 2310/3181; B01J 14/00

(57) ABSTRACT

This invention pertains to the field of biopolymer-loaded (where the biopolymer(s) are active pharmaceutical ingredient(s) (API(s)) polymer-based nanoparticles formulated, for example, for curative, therapeutic prophylactic and/or diagnostic applications. The polymer used to formulate the nanoparticles can be any biodegradable synthetic polymer or combination of polymers, including combinations of PLGA and PEG. Biopolymers used as active pharmaceutical ingredients (API) can include natural and unnatural nucleic acids such as DNA, RNA and LNA. Biopolymers used as active pharmaceutical ingredients (APIs) can also include neutral and positively charged nucleic acid mimics (NPNAM) such as for example, peptide nucleic acids, morpholinos, pyrrolidine-amide oligonucleotide mimics, morpholinoglycine oligonucleotides and methyl phosphonates and derivatives thereof. In some embodiments, the nanoparticles are loaded with both nucleic acids and NPNAMs as the APIs. Nanoparticles so formulated can be used in curative, therapeutic, prophylactic and/or diagnostic applications. Certain preferred nanoparticles, formulation methodologies, de-formulation methodologies and apparatus are also disclosed.

### CROSS REFERENCE TO RELATED APPLICATIONS

This application claims the benefit of U.S. Provisional Patent application Ser. No. 62/615,389, filed on Jan. 9, 2018, and U.S. Provisional Patent application Ser. No. 62/696,791 filed on Jul. 11, 2018, both of which are incorporated herein by reference.

### SEQUENCE LISTING

The instant application contains a Sequence Listing which has been submitted electronically in ASCII format and is hereby incorporated by reference in its entirety. Said ASCII copy, created on Mar. 18, 2019, is named T2110-700910_SL.txt and is 3,001 bytes in size.

The section headings used herein are for organizational purposes only and should not be construed as limiting the subject matter described in any way.

### BRIEF DESCRIPTION OF DRAWINGS

The skilled artisan will understand that the drawings, described below, are for illustration purposes only. The drawings are not intended to limit the scope of the present teaching in any way. Drawings are not necessarily presented in any scale and should not be interpreted as implying any scale. In various figures and chemical formulas, a point of attachment to another moiety is sometimes illustrated for clarity.

FIG. 1 is an illustration that provides nomenclature of a peptide nucleic acid (PNA).

FIG. 2 is an illustration that provides the anatomy of a tail clamp molecule such as a tail clamp PNA (tcPNA) (e.g., SEQ ID NO: 12).

FIG. 3 is an illustration depicting the process of forming nanoparticles by double emulsion (a.k.a. batch mixing) methodology.

FIG. 4 is an exemplary flow diagram of a process(es) described herein for manufacturing nanoparticles with encapsulated/entrapped neutral or positively charged nucleic acid mimics (NPNAM) such as peptide nucleic acids.

FIG. 5 is an illustration of one possible exemplary embodiment of an apparatus for carrying out the process(es) described herein and illustrated in FIG. 4 for the manufacturing of nanoparticles with encapsulated/entrapped neutral or positively charged nucleic acid mimics (NPNAM).

FIG. 6A is a graphical result for the size distribution of one exemplary batch of PLGA nanoparticles made using the process(es) described herein and having an average size of about 35 nanometers.

FIG. 6B is a graphical result for the size distribution of one exemplary batch of PLGA/PEG nanoparticles made using the process(es) described herein and having an average size of about 60 nanometers.

FIG. 6C is a graphical result for the size distribution of one exemplary batch of PLGA nanoparticles made using the process(es) described herein and having an average size of about 190 nanometers.

FIG. 6D is an overlay of the graphical results shown in FIGS. 6A-6C.

FIG. 6E is a graphical result for the size distribution of one batch of PLGA nanoparticles made using the process(es) described herein and used in the gene editing experiments described in Example 3, below.

FIG. 7A is a graphical result for the size distribution of three different exemplary batches of PLGA/PEG nanoparticles made using the process(es) described herein, wherein in each case, the concentration of PLGA/PEG was 5 mg/mL, 10 mg/mL or 20 mg/mL.

FIG. 7B is a graphical result for the size distribution of three different exemplary batches of PLGA/PEG nanoparticles made using the process(es) described herein, wherein in each case, one of either dimethylsulfoxide (DMSO), acetone or acetonitrile was used as the organic solvent.

FIG. 8A is graphical result for the size distribution of three different batches of PLGA/PEG nanoparticles made using the process(es) described herein, wherein in each case, one of either dimethylsulfoxide (DMSO), acetone or acetonitrile was used as the organic solvent.

FIG. 8B is graphical result for the size distribution of the same exemplary batch of PLGA/PEG nanoparticles made using the process(es) described herein, wherein the nanoparticles have been examined for size: (i) after initial formation; (ii) after lyophilization; and (iii) again after being frozen and thawed.

FIG. 8C is graphical result for the size distribution of two different batches of PLGA/PEG nanoparticles made using the process(es) described herein, wherein in each case, the nanoparticles are formulated with a different PNA/DNA combination.

FIGS. 9A-9E are images and related data from a CryoTEM analysis of exemplary PLGA nanoparticles loaded with PNA and DNA made using the process(es) described herein and used in the experiments described in Example 3. FIGS. 9A-9B are images at medium magnification; FIG. 9C is an image at high magnification; FIG. 9D is an image showing representative detected nanoparticles at low magnification; and FIG. 9E is a size distribution histogram of exemplary particles showing the related size distribution statistics for the particle examined.

FIG. 10 is a graphical result for the zeta potential of one exemplary batch of PLGA nanoparticles made using this process wherein the organic solvent is acetone and the nanoparticles have a zeta potential of about −20.

FIG. 11A is an image of exemplary data obtained for digital drop PCR (ddPCR) analysis of a no target control (NTC) and a sample of bone marrow cells from a mouse (i.e. “mBM”) that have been treated in vitro with nanoparticles prepared using the process(es) disclosed herein and containing PNA and donor DNA that is designed to genetically edit the bone marrow cells to correct the sickle cell disease (SCD) causing mutation wherein the ddPCR analysis is used to determine percent editing of the SCD mutation in the treated sample.

FIG. 11B is graphically presented data summarizing gene editing results obtained by ddPCR for four different samples of mouse bone marrow cells treated in vitro wherein one sample received empty PLGA nanoparticles and the other three samples were treated with PLGA nanoparticles prepared using the process(es) disclosed herein and containing PNA and donor DNA that is designed to genetically edit the bone marrow cells to correct the sickle cell disease (SCD) causing mutation.

FIG. 12A is graphically presented data summarizing gene editing results collected in a dose-dependent study of a human B-cell line that is homozygous for the sickle cell mutation wherein the cells were treated in vitro with PLGA nanoparticles prepared using the process(es) disclosed herein and containing PNA and donor DNA that is designed to correct the sickle cell disease (SCD) causing mutation.

FIG. 12B is graphically presented data summarizing gene editing results collected for a time-course study of a human B-cell line that is homozygous for the sickle cell mutation wherein the cells were treated in vitro with PLGA nanoparticles prepared using the process(es) disclosed herein and containing PNA and donor DNA that is designed to correct the sickle cell disease (SCD) causing mutation.

FIG. 12C is graphically presented data summarizing gene editing results collected for a repeated-treatment study of a human B-cell line that is homozygous for the sickle cell mutation wherein the cells were treated in vitro with PLGA nanoparticles prepared using the process(es) disclosed herein and containing PNA and donor DNA that is designed to correct the sickle cell disease (SCD) causing mutation.

FIG. 13A is graphically presented data summarizing gene editing results collected for sickle cell patient peripheral blood mononuclear cells (PBMCs) wherein the PBMCs were treated in vitro with PLGA nanoparticles prepared using the process(es) disclosed herein and containing PNA and donor DNA that is designed to correct the sickle cell disease (SCD) causing mutation.

FIG. 13B is graphically presented data summarizing gene editing results collected for CD34+ cells of sickle cell patients from patient PBMCs, wherein the CD34+ cells were treated in vitro with PLGA nanoparticles prepared using the process(es) disclosed herein and containing PNA and donor DNA that is designed to correct the sickle cell disease (SCD) causing mutation.

FIG. 14A is graphically presented data summarizing gene editing results for editing/correction of SCD in mouse cells treated in vitro with PLGA nanoparticles prepared using the process(es) disclosed herein and containing PNA and donor DNA that is designed to correct the sickle cell disease (SCD) causing mutation, wherein the comparison is between percent editing as determined either by ddPCR or by next generation sequencing (NGS).

FIG. 14B is graphically presented data summarizing gene editing results for editing/correction of human SCD patient cells treated in vitro with PLGA nanoparticles prepared using the process(es) disclosed herein and containing PNA and donor DNA that is designed to correct the sickle cell disease (SCD) causing mutation, wherein the comparison is between percent editing as determined either by ddPCR or by next generation sequencing (NGS).

FIG. 14C is graphically presented data summarizing gene editing results for editing/correction of a SCD cell line treated in vitro with PLGA nanoparticles prepared using the process(es) disclosed herein and containing PNA and donor DNA that is designed to correct the sickle cell disease (SCD) causing mutation, wherein the comparison is between percent editing as determined either by ddPCR or by next generation sequencing (NGS).

FIG. 15A is graphically presented data summarizing gene editing results collected for human SCD patient cells treated in vitro with freshly made PLGA nanoparticles prepared using the process(es) disclosed herein and containing PNA and donor DNA that is designed to correct the sickle cell disease (SCD) causing mutation.

FIG. 15B is graphically presented data summarizing gene editing results collected for the same human SCD patient cells treated in vitro with the PLGA nanoparticles and protocol used to produce the data in FIG. 15A, but where the PLGA nanoparticles had been stored for 3 weeks at 4° C.

FIG. 16 is graphically presented data summarizing gene editing results collected for human SCD patient cells treated in vitro with PLGA nanoparticles prepared using the process(es) disclosed herein and containing PNA and donor DNA that is designed to correct the sickle cell disease (SCD) causing mutation, wherein the PLGA nanoparticles were subjected to one of three different storage conditions (i.e. Frozen, Lyophilized or Suspended) for 24 hours prior to use.

All literature and similar materials cited in this application, including but not limited to patents, patent applications, articles, books and treatises, regardless of the format of such literature or similar material, are expressly incorporated by reference herein in their entirety for any and all purposes.

### DESCRIPTION

### 1. Field

This invention pertains to the field of polymer-based nanoparticles comprising neutral or positively-charged nucleic acid mimics (NPNAM)s, wherein the NPNAM(s) can be active pharmaceutical ingredient(s) (API(s)). In some embodiments, the NPNAM-loaded polymer-based nanoparticles may be used, inter alia, for curative, therapeutic and/or prophylactic applications. The nanoparticles so formed may comprise only one or more NPNAMs or may comprise both one or more NPNAMs as well as one or more nucleic acids, for example, as APIs.

### 2. Introduction

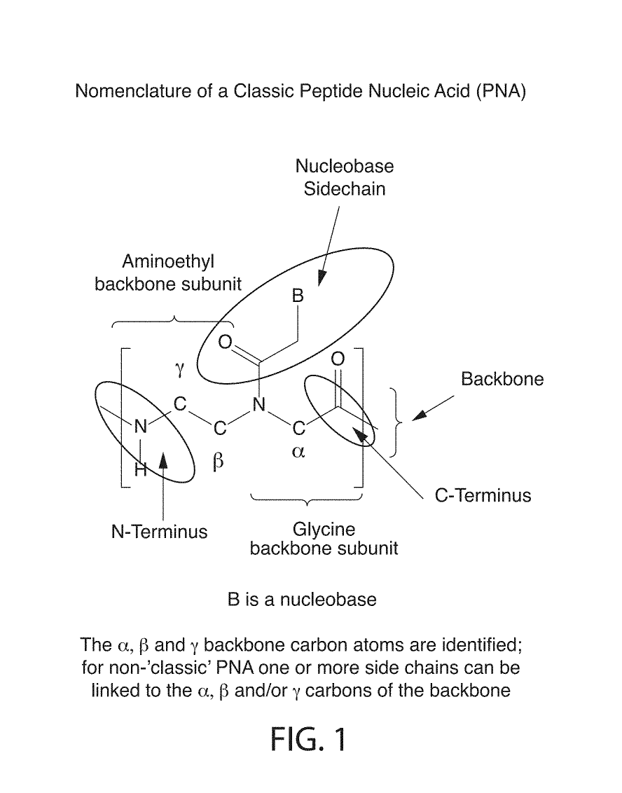

Peptide Nucleic Acids (PNAs) are non-naturally occurring polyamides that can hybridize to nucleic acids (DNA and RNA) with sequence specificity. (See U.S. Pat. No. 5,539,082 and Egholm et al., Nature (1993) 365, 566-568). Despite its name, a peptide nucleic acid is neither a peptide, nor is it a nucleic acid. Classic PNAs are achiral synthetic polyamides (not occurring in nature) of repeating 2-(aminoethyl)glycine subunits comprising linked nucleobases that sequence specifically hybridize to nucleic acid to form hybrids that are generally more thermodynamically stable than a corresponding nucleic acid/nucleic acid complex (Egholm et al., supra). In addition to the ‘classic’ PNA, its inventors contemplated appending amino acid side chains as branches to the α, β and γ positions of the PNA polymer backbone (See: FIG. 1 and U.S. Pat. No. 5,539,082). Various other modifications of PNA have been described in the literature, probably the most notable being the so-called gamma miniPEG PNAs (See: Sahu et al., Synthesis and Characterization of Conformationally Preorganized, γ(R)-Diethylene Glycol-Containing—Peptide Nucleic Acids with Superior Hybridization Properties and Water Solubility, JOC, 76: 5614-5627 (2011)). Being non-naturally occurring molecules, PNAs are not known to be substrates for the enzymes that are known to degrade peptides or nucleic acids. Because they are polyamides, PNAs may be synthesized by adaptation of standard peptide synthesis procedures, including use of automated synthesizers.

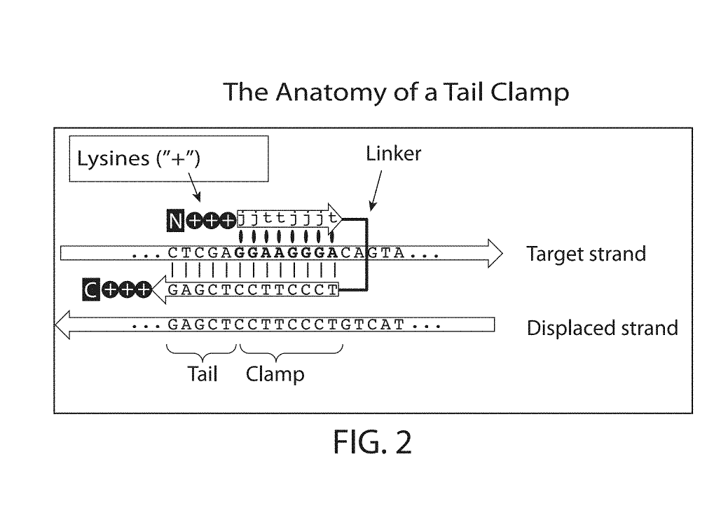

In recent years, much effort and investment has been made in development of CRISPR Cas 9 for gene editing. However, a strictly chemical approach to gene editing has also been shown to cause a phenotypic correction in mice (See: Bahal et al., In vivo correction of anemia in β-thalassemic mice by γPNA-mediated gene editing with nanoparticle delivery, Nat. Commun, 7: 13304, Oct. 26, 2016). In this report, the authors use a nanoparticle to encapsulate/entrap both a donor DNA and a tail-clamp PNA oligomer (See: FIG. 2 for the anatomy of a tail-clamp and also See: Bentin et al., Combined Triplex/Duplex Invasion of Double Stranded DNA by “Tail-Clamp” Peptide Nucleic Acid, Biochemistry, 42: 13987-13995 (2003) and Kaihatsu et al. Extended Recognition by Peptide Nucleic Acids (PNAs): Binding to Duplex DNA and Inhibition of Transcription by Tail-Clam PNA-Peptide Conjugates, Biochemistry, 42: 13996-14002 (2003) for a broader discussion of tail-clamp PNAs (tcPNAs)), wherein the nanoparticle is able to effect cellular delivery of the donor DNA and tcPNA molecules. The donor DNA and tcPNA are designed to cause repair of a genetic defect that causes disease (in this case R-thalassemia). The donor DNA and tcPNA were encapsulated in nanoparticles formed from poly(lactic-co-glycolic acid) (PLGA) using a double emulsion process.

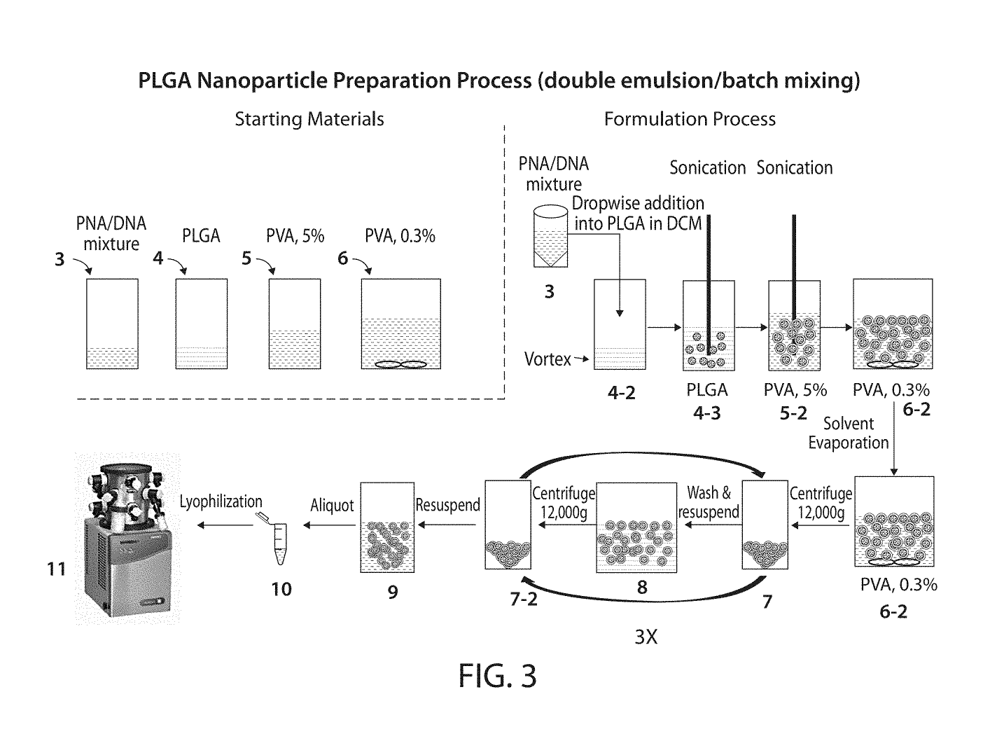

With reference to FIG. 3, the double emulsion process for preparing nanoparticles comprising entrapped/encapsulated PNA and DNA suitable for gene editing as described by Bahal et al., is illustrated. As illustrated, a mixture of DNA and PNA in aqueous buffer 3 is provided. Also prepared is a stock solution of synthetic polymer (e.g. PLGA) in a water immiscible organic solvent (e.g. dichloromethane) 4, a solution containing polyvinyl alcohol (PVA) in a high concentration (5%) 5 in an aqueous solution as compared with a second solution comprising PVA at a desired lower concentration (0.3%) in aqueous solution 6. As illustrated, these solutions are considered the Starting Materials for the Formulation Process.

Generally, the double emulsion process entails adding a mixture of DNA and PNA in aqueous solution 3 dropwise to the mixture of synthetic polymer dissolved in the water immiscible organic solvent 4-2, along with vortexing and then sonication as necessary to form an emulsion 4-3. The contents of 4-3 (the emulsion) are then added to the concentrated solution of surfactant 5 to produce a solution comprising organic solvent, PVA, aqueous solvent/buffer and nanoparticles 5-2. The contents of 5-2 are then transferred to the solution comprising the dilute concentration of PVA in aqueous solvent/buffer to thereby produce the nanoparticles in final form 6-2.

The nanoparticles as formed can then be extracted for analysis/quality control if so desired. Otherwise, the solution can be transferred to a centrifuge tube and spun and then decanted to produce the desired nanoparticles 7. In order to remove any residual organic solvent, PVA and/or buffer, the nanoparticles can be put through one or more cycles of washing by resuspension in an aqueous wash solution 8, followed by centrifugation to re-pellet the nanoparticles 7-2. When washed to a level of purity that is desired, the nanoparticles can be resuspended in a solution 9, and if desired, the batch can be split into portions by aliquoting into separate containers (e.g. an Eppendorf tube) 10. Whether or not split into portions, the nanoparticles can then be prepared for long term storage by lyophilization 11.

The aforementioned “double emulsion” process suffers from some batch-to-batch inconsistency in loading of API (i.e. DNA and PNA), as well as an inability to scale. For these reasons, improved methodologies are desired to make NPNAM-loaded nanoparticles readily available for uses that can include inter alia, curative, therapeutic and/or prophylactic applications.

### 3. Definitions

For the purposes of interpreting of this specification, the following definitions will apply and whenever appropriate, terms used in the singular will also include the plural and vice versa. In the event that any definition set forth below conflicts with the usage of that word in any other document, the definition set forth below shall always control for purposes of interpreting the scope and intent of this specification and its associated claims. Notwithstanding the foregoing, the scope and meaning of a word contained any document incorporated herein by reference should not be altered (for purposes of interpreting said document) by the definition presented below. Rather, said incorporated document (and words found therein) should be interpreted as it/they would be by the ordinary practitioner at the time of its publication based on its content and disclosure and when considered in terms of the context of the content of the description provided herein.

### a. General Definitions

The use of “or” means “and/or” unless stated otherwise or where the use of “and/or” is clearly inappropriate. The use of “a” means “one or more” unless stated otherwise or where the use of “one or more” is clearly inappropriate. The use of “comprise,” “comprises,” “comprising”, “having”, “include,” “includes,” and “including” are interchangeable and not intended to be limiting (i.e. open ended). Furthermore, where the description of one or more embodiments uses the term “comprising,” those skilled in the art would understand that in some specific instances, the embodiment or embodiments can be alternatively described using language “consisting essentially of” and/or “consisting of.”

As used herein, the symbol “ [symbol] ” cutting across a bond indicates the bond that is a point of attachment of the moiety illustrated to another atom, moiety or chemical structure (or subcomponent thereof).

As used herein, the term “biopolymer’ refers to NPNAM(s) and nucleic acids (including, without limitation, locked nucleic acids (LNAs)).

As used herein, the term “deformulation” refers to breaking down a formulated nanoparticle in a manner that permits analysis of at least its encapsulated active ingredients such as encapsulated/entrapped NPNAM(s) and, if applicable, nucleic acid components.

As used herein, the term “diafiltration” refers to a dilution/filtration process for completely removing, replacing, and/or lowering the concentration of components in a solution (e.g. salts, nucleic acids, NPNAMs, small proteins, solvents etc.) in order to obtain a solution with higher purity in some regard, wherein such components can be considered as impurities of a solution comprising the nanoparticles.

As used herein, the term “gene targeting composition” means a composition (e.g. a nanoparticle) comprising a NPNAM capable of altering a nucleic acid. In some embodiments, altering a nucleic acid comprises one or more of the following:
a) altering the state of association of the two strands of a target double stranded nucleic acid;
b) altering the helical structure of a target double stranded nucleic acid;
c) altering the topology in a strand of a target double stranded nucleic acid (e.g., introducing a kink or bend in a strand of the target double stranded nucleic acid);
d) recruiting a nucleic acid-modifying protein (e.g., a nucleic acid-modifying enzyme), for example, a member of the nucleotide excision repair pathway, to a target double stranded nucleic acid. Exemplary members of the nucleotide excision repair pathway include XPA, RPA, XPF, and XPG, or a functional variant or fragment thereof;
e) cleaving a strand of a target double stranded nucleic acid; or
f) altering the sequence of a target double stranded nucleic acid. In some embodiments, the sequence of a target double stranded nucleic acid is altered to the sequence of a template nucleic acid. In some embodiments, the sequence of a target double stranded nucleic acid is altered from a mutant or disorder-associated sequence (e.g., allele) to a non-mutant or non-disease associated sequence (e.g., allele).

In some embodiments, a gene targeting composition is used in vitro. In some embodiments, a gene targeting composition is used in vivo. In some embodiments, a gene targeting composition further comprises a load component (e.g., a nucleic acid, e.g., a nucleic acid that targets the sequence of a target double stranded nucleic acid, e.g., a template nucleic acid). A gene targeting composition may comprise a gene editing composition.

As used herein, a “gene editing composition” refers to a composition comprising a NPNAM capable of editing a nucleic acid. In some embodiments, when placed within an animal (e.g. by injection or otherwise), a gene editing composition is capable of editing the genome of a cell in the animal. For clarity, gene editing compositions may also encompass multi-component systems where different components of the gene editing composition are delivered individually (e.g. different APIs used in combination are delivered individually to the subject). In some embodiments, the NPNAM of the gene editing composition is packaged into a single composition of matter for delivery to the subject (e.g. all APIs of the gene editing composition are loaded into a single nanoparticle). In some embodiments, a gene editing composition further comprises a load component (e.g., a nucleic acid, e.g., a nucleic acid that targets the sequence of a target double stranded nucleic acid, e.g., a template nucleic acid).

As used herein, the terms “Hoogsteen binding” and ‘Hoogsteen base-pairing” are synonymous and refer to a non-canonical/non-Watson-Crick hydrogen-bonded motif wherein a polymeric strand comprising a nucleobase sequence specifically hydrogen bonds to a double stranded duplex via contacts in the major groove.

As used herein, “linker” refers to a chemical moiety that links, e.g., covalently, at least two other atoms, groups, residues, segments or moieties. For example, a linker can link two PNA residues together or two PNA oligomer segments together, such as a linker used in a tail-clamp PNA (tcPNA—See for example FIG. 2 as an illustration of a tcPNA). PEG3 is an example of a linker that can be used to link together two distinct segments of consecutively linked PNA subunits.

As used herein, the abbreviation “NPNAM” refers to a neutral or positively charged nucleic acid mimic. For clarity, a NPNAM (as used herein) can comprise negatively charged groups or subunits so long as the net charge of the biopolymer is neutral or positive. In some embodiments, a NPNAM is a peptide nucleic acid (PNA), e.g., a tail-clamp PNA. In some embodiments, a NPNAM is a PNA oligomer comprising the structure of Formula (I), e.g., as described herein.

As used herein, the term “nucleic acid mimic” refers to a non-naturally occurring polymer composition that nevertheless possesses the ability to sequence-specifically hybridize to nucleic acid. Some non-limiting examples of nucleic acid mimics include peptide nucleic acids (PNAs—including all forms of PNAs as described in more detail herein), morpholinos (also known as PMOs, See: U.S. Pat. Nos. 5,142,047 and 5,185,444), pyrrolidine-amide oligonucleotide mimics (POMs—See: T. H. Samuel Tan et al., Org. Biomol. Chem., 5: 239-248 (2007), morpholinoglycine oligonucleotides (MGOs—See: Tatyana V. et al., Beilstein J Org Chem. 10: 1151-1158 (2014)), and methyl phosphonates. In some embodiments, a nucleic acid mimic is a neutral or positively charged nucleic acid mimic (NPNAM).

As used herein, the term “nucleobase” refers to those naturally occurring and those non-naturally occurring heterocyclic moieties known to those who utilize nucleic acid technology or utilize peptide nucleic acid technology to thereby generate polymers that sequence-specifically hybridize/bind to nucleic acids by any means, including without limitation through Watson-Crick and/or Hoogsteen binding motifs. A non-limiting list of nucleobases includes: adenine, guanine, thymine, cytosine, uracil, pseudoisocytosine, 2-thiopseudoisocytosine, 5-methylcytosine, 5-hydroxymethyl cytosine, xanthine, hypoxanthine, 2-aminoadenine (a.k.a. 2,6-diaminopurine), 2-thiouracil, 2-thiothymine, 2-thiocytosine, 5-chlorouracil, 5-bromouracil, 5-iodouracil, 5-chlorocytosine, 5-bromocytosine, 5-iodocytosine, 5-propynyl uracil, 5-propynyl cytosine, 6-azo uracil, 6-azo cytosine, 6-azo thymine, 7-methylguanine, 7-methyladenine, 8-azaguanine, 8-azaadenine, 7-deazaguanine, 7-deazaadenine, 7-deaza-2,6-diaminopurine, 3-deazaguanine, 3-deazaadenine, 7-deaza-8-aza guanine, 7-deaza-8-aza adenine, and 2-thio-5-propynyl uracil, including tautomeric forms of any of the foregoing.

As used herein, the term “peptide nucleic acid” or the abbreviation “PNA” refers to a non-natural polymer composition comprising linked nucleobases capable of sequence specifically hybridizing to nucleic acid. Exemplary PNAs are disclosed in or otherwise claimed in any of the following: U.S. Pat. Nos. 5,539,082, 5,527,675, 5,623,049, 5,714,331, 5,736,336, 5,773,571 or 5,786,461; (each of the foregoing are herein incorporated herein by reference in its entirety). The term “peptide nucleic acid” or “PNA” shall also apply to polymers comprising two or more subunits of kind described in the following publications: Diderichsen et al., Tett. Lett. 37: 475-478 (1996); Fujii et al., Bioorg. Med. Chem. Lett. 7: 637-627 (1997); Jordan et al., Bioorg. Med. Chem. Lett. 7: 687-690 (1997); Krotz et al., Tett. Lett. 36: 6941-6944 (1995); Lagriffoul et al., Bioorg. Med. Chem. Lett. 4: 1081-1082 (1994); Lowe et al., J. Chem. Soc. Perkin Trans. 1, (1997) 1:539-546; Lowe et al., J. Chem. Soc. Perkin Trans. 11: 547-554 (1997); Lowe et al., J. Chem. Soc. Perkin Trans. 11:5 55-560 (1997); Petersen et al., Bioorg. Med. Chem. Lett. 6: 793-796 (1996); Diederichsen, U., Bioorganic & Med. Chem. Lett., 8: 165-168 (1998); Cantin et al., Tett. Lett., 38: 4211-4214 (1997); Ciapetti et al., Tetrahedron, 53: 1167-1176 (1997); Lagriffoule et al., Chem. Eur. J., 3: 912-919 (1997); WIPO patent application WO96/04000 by Shah et al. and entitled “Peptide-based nucleic acid mimics (PENAMs)”; phosphono-PNA analogues (pPNAs) as described in: Van der Laan A. C. et al., Tetrahedron Lett. 37: 7857-7860 (1996); trans-4-hydroxy-L-proline nucleic acids (HypNAs) as described in Efimov et al., Hydroxyproline-based DNA mimics provide and efficient gene silencing in vitro and in vivo, Nucleic Acids Res. 34(8): 2247-2257 (2006); and (1S,2R/1R,2S)-cis-cyclopentyl PNAs (cpPNAs) as described in Govindaraju, T. et al., J Org Chem. 69(17): 5725-34 (2004); each of the foregoing are herein incorporated herein by reference in its entirety.

In some embodiments, as used herein, “PNA oligomer” refers to any polymeric composition of matter comprising two or more PNA residues. For example, a PNA oligomer can comprise two or more subunits of formula XX (for example as shown in formula XXI):

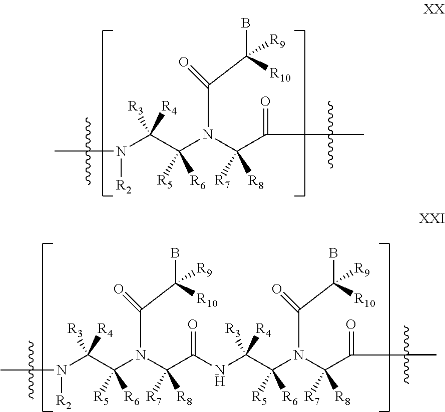

wherein, B is a nucleobase and the moieties: R₂, R₃, R₄, R₅, R₆, R₇, R₈, R₉ and R₁₀ are as defined below wherein the points of attachment of the subunit within the polymer are as illustrated. In some embodiments, the PNA subunits are directly linked to one or more other PNA subunits. In some embodiments, the two or more PNA subunits are not directly linked to another PNA subunit. In some embodiments, two or more PNA subunits of the polymer are linked together by a linker such that the polymer can contain a consecutively-linked stretch of PNA subunits (a ‘segment’) interrupted by a linker followed by another stretch of consecutively-linked PNA subunits (e.g., a second ‘segment). An example of such a PNA oligomer is a tail-clamp PNA or tcPNA (defined below). A PNA oligomer can be a chimeric molecule, for example, a PNA-DNA chimera as described in U.S. Pat. No. 6,063,569.

Specifically, moiety R₂ can be hydrogen (H), deuterium (D) or C₁-C₄ alkyl; each of R₃, R₄, R₅, R₆, R₇ and R₈ can be independently selected from the group consisting of: H, D, F, and a side chain selected from the group consisting of: IIIa, IIIb, IIIc, IIId, IIIe, IIIf, IIIg, IIIh, IIIi, IIIj, IIIk, IIIm, IIIn, IIIo, IIIp, IIIq, IIIr, IIIs, IIIt, IIIu, IIIy, IIIw, IIIx, IIIy, IIIz, IIIaa and IIIab, wherein each of IIIi, IIIj, IIIk, IIIm, IIIn, IIIo, IIIp, IIIq, IIIr, IIIs, IIIt, IIIu, IIIy, IIIw, IIIx, IIIy, and IIIz independently and optionally comprise a protecting group;

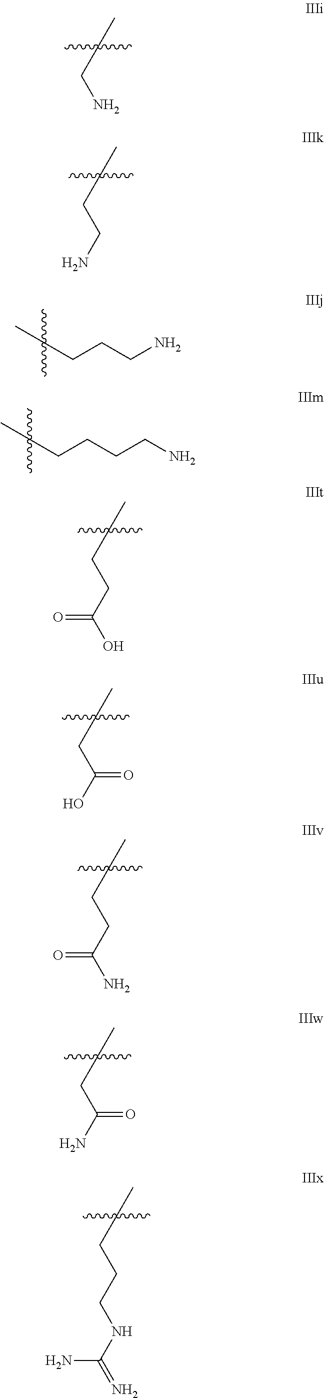

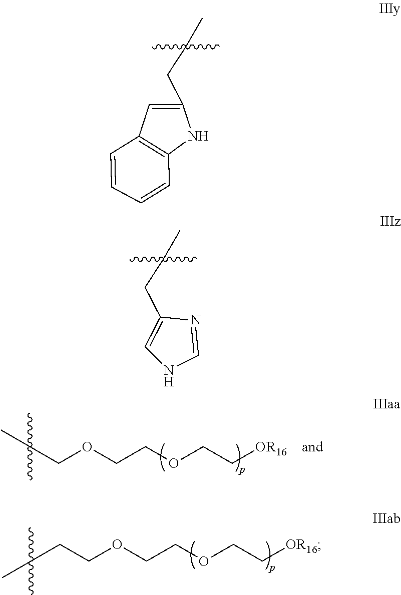

each of R₉ and R₁₀ can be independently selected from the group consisting of: H, D, and F; R₁₆ can be selected from H, D and C₁-C₄ alkyl group; and p can be a whole number from 0 to 10, inclusive.

As used herein the terms “residue” and “subunit” are interchangeable and refer to the part of a PNA monomer, amino acid, linker, label, building block or other moiety that becomes incorporated into a polymer, such as a PNA oligomer, polyamide or oligonucleotide during chemical or enzymatic assembly.

As used herein, “segment” or “segments” refers to a string of consecutively-linked monomer subunits (residues).

As used herein, the term “synthetic polymer’ refers to a non-naturally occurring polymer (including without limitation any form of polymer conjugate, co-polymer, block polymer, block co-polymer, polymer mixture and/or polymer blend). In some embodiments, a synthetic polymer as used herein is capable of encapsulating/entrapping a NPNAM or other payload into a nanoparticle upon formulation. Copolymers can be random, block or comprise a combination of random and block sequences. The repeat units forming the copolymer may be arranged in any order. In some embodiments, a synthetic polymer comprises a structure of Formula (II), described herein. Exemplary synthetic polymers include polylactic acid (PLA) polyglycolic acid, (PGA), poly(lactic-co-glycolic acid) (PLGA), poly(4-hydroxy-L-proline ester), other degradable polyesters, polyanhydrides, poly(ortho)esters, polyesters, polyurethanes, poly(butic acid), poly(valeric acid), poly(caprolactone), poly(hydroxyalkanoates), poly(lactide-co-caprolactone), poly(amine-co-ester) polymers or PEG polymers, or a combination of any two or more of the foregoing, including pegylated versions thereof and wherein the polymer can be a polymer conjugate, a co-polymer, a block polymer, a polymer mixture and/or a polymer blend. Compositions and particles disclosed may or may not contain a water-soluble polymer conjugate that can endow the particle with improved properties, e.g., steric stability. One such water-soluble polymer commonly used is polyethyleneglycol (PEG). Attachment of a PEG moiety to a polymer (e.g., to the terminal end of the polymer) results in formation of a “pegylated polymer” or a “PEG-polymer.” Exemplary synthetic PEG-polymers include PEG-polylactic acid (PLA), PEG-polyglycolic acid, (PGA), PEG-poly(lactic-co-glycolic acid) (PLGA), PEG-poly(4-hydroxy-L-proline ester), other degradable PEG-polyesters, PEG-polyanhydrides, PEG-poly(ortho)esters, PEG-polyesters, PEG-polyurethanes, PEG-poly(butic acid), PEG-poly(valeric acid), PEG-poly(caprolactone), PEG-poly(hydroxyalkanoates), PEG-poly(lactide-co-caprolactone), PEG-poly(amine-co-ester) polymers, or a combination of any two or more of the foregoing or derivatives thereof. Pegylated polymers may include pegylated monomers such as PEG-PLA or pegylated copolymers such as PEG-PLGA. It is appreciated that other water soluble-polymers may also be used to conjugate to a synthetic polymer and used in such nanoparticles such as HPMA (poly[N-(2-hydroxypropyl) methacrylamide]), PVP (poly(vinylpyrrolidone)), PMOX (poly(2-methyl-2-oxazoline)), PDMA (poly(N,N-dimethyl acrylamide)), and PAcM (poly(N-acryloyl morpholine)). A synthetic polymer may be chiral or achiral. If the synthetic polymer comprises one or more chiral centers, the synthetic polymer can be racemic or can be enriched in any state of enantiomeric enrichment. If applicable, the synthetic polymer can adopt any tautomeric form.

As used herein, “tail-clamp” or “tcPNA”, refers to a PNA oligomer capable of forming a PNA/DNA/PNA triplex upon binding to a target nucleic acid sequence (e.g., a target double stranded DNA sequence). A tcPNA comprises: i) a first segment comprising a plurality of PNA residues that participate in binding to the Hoogsteen face of a nucleic acid sequence; and ii) a second segment comprising a plurality of PNA residues that participate in binding to the Watson-Crick face of the nucleic acid sequence. In an embodiment, the first segment and second segment can be linked by a linker (e.g., a polyethylene glycol-based linker such as PEG3). A tcPNA may further comprise: iii) a third segment comprising a plurality of PNA resides; and/or iv) a positively charged region comprising a plurality of positively charged moieties (e.g., positively charged amino acids such as lysine or arginine) which may be present on a terminus of the tcPNA. An exemplary tcPNA is depicted bound to a dsDNA is illustrated in FIG. 2. In some embodiments, a tcPNA can comprise three lysine residues at the N-terminus and three lysine residues at the C-terminus (See legend to illustration in FIG. 2).

As used herein, “Watson-Crick binding” refers to the well-established motif whereby Watson-Crick base pairs (guanine-cytosine and adenine-thymine (uracil for RNA)) allow a double stranded nucleic acid to form a helix and maintain a regular helical structure that is subtly dependent on its nucleotide sequence. The complementary nature of this based-paired structure provides a backup copy of all genetic information encoded within double-stranded nucleic acid. Notwithstanding the foregoing, as used herein, “Watson-Crick binding” is also intended to encompass other base-pair motifs that are designed to allow a double stranded nucleic acid to form a helix and maintain a regular helical structure that is subtly dependent on its nucleotide sequence.

### b. Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

The abbreviations used herein have their conventional meaning within the chemical and biological arts. The chemical structures and formulae set forth herein are constructed according to the standard rules of chemical valency known in the chemical arts.

When a range of values is listed, it is intended to encompass each value and sub-range within the range. For example “C₁-C₆ alkyl” is intended to encompass, C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl.

The following terms are intended to have the meanings presented therewith below and are useful in understanding the description and intended scope of the present invention.

As used herein, “alkyl” refers to a radical of a straight-chain or branched saturated hydrocarbon group having from 1 to 12 carbon atoms (“C₁-C₁₂ alkyl”). In some embodiments, an alkyl group has 1 to 10 carbon atoms (“C₁-C₁₀ alkyl”). In some embodiments, an alkyl group has 1 to 8 carbon atoms (“C₁-C₈ alkyl”). In some embodiments, an alkyl group has 1 to 6 carbon atoms (“C₁-C₆ alkyl”). In some embodiments, an alkyl group has 1 to 5 carbon atoms (“C₁-C₅ alkyl”). In some embodiments, an alkyl group has 1 to 4 carbon atoms (“C₁-C₄alkyl”). In some embodiments, an alkyl group has 1 to 3 carbon atoms (“C₁-C₃ alkyl”). In some embodiments, an alkyl group has 1 to 2 carbon atoms (“C₁-C₂ alkyl”). In some embodiments, an alkyl group has 1 carbon atom (“C₁ alkyl”). In some embodiments, an alkyl group has 2 to 6 carbon atoms (“C₂-C₆ alkyl”). Examples of C₁-C₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Each instance of an alkyl group may be independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In certain embodiments, the alkyl group is substituted C₁₋₆ alkyl.

As used herein, “alkenyl” refers to a radical of a straight-chain or branched hydrocarbon group having from 2 to 12 carbon atoms, one or more carbon-carbon double bonds, and no triple bonds (“C₂-C₁₂ alkenyl”). In some embodiments, an alkenyl group has 2 to 10 carbon atoms (“C₂-C₁₀ alkenyl”). In some embodiments, an alkenyl group has 2 to 8 carbon atoms (“C₂-C₈ alkenyl”). In some embodiments, an alkenyl group has 2 to 6 carbon atoms (“C₂-C₆ alkenyl”). In some embodiments, an alkenyl group has 2 to 5 carbon atoms (“C₂-C₅ alkenyl”). In some embodiments, an alkenyl group has 2 to 4 carbon atoms (“C₂-C₄ alkenyl”). In some embodiments, an alkenyl group has 2 to 3 carbon atoms (“C₂-C₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂-C₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂-C₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Each instance of an alkenyl group may be independently optionally substituted, ie., unsubstituted (an “unsubstituted alkenyl”) or substituted (a “substituted alkenyl”) with one or more substituents e.g., for instance from 1 to 5 substituents, 1 to 3 substituents, or 1 substituent. In certain embodiments, the alkenyl group is unsubstituted C₂₋₁₀ alkenyl. In certain embodiments, the alkenyl group is substituted C₂₋₆ alkenyl.

As used herein, the term “alkynyl” refers to a radical of a straight-chain or branched hydrocarbon group having from 2 to 12 carbon atoms, one or more carbon-carbon triple bonds (“C₂-C₁₂ alkynyl”). In some embodiments, an alkynyl group has 2 to 10 carbon atoms (“C₂-C₁₀ alkynyl”). In some embodiments, an alkynyl group has 2 to 8 carbon atoms (“C₂-C₈ alkynyl”). In some embodiments, an alkynyl group has 2 to 6 carbon atoms (“C₂-C₆ alkynyl”). In some embodiments, an alkynyl group has 2 to 5 carbon atoms (“C₂-C₅ alkynyl”). In some embodiments, an alkynyl group has 2 to 4 carbon atoms (“C₂-C₄ alkynyl”). In some embodiments, an alkynyl group has 2 to 3 carbon atoms (“C₂-C₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂-C₄ alkynyl groups include ethynyl (C₂), 1-propynyl (C₃), 2-propynyl (C₃), 1-butynyl (C₄), 2-butynyl (C₄), and the like. Each instance of an alkynyl group may be independently optionally substituted, ie., unsubstituted (an “unsubstituted alkynyl”) or substituted (a “substituted alkynyl”) with one or more substituents e.g., for instance from 1 to 5 substituents, 1 to 3 substituents, or 1 substituent. In certain embodiments, the alkynyl group is unsubstituted C₂₋₁₀ alkynyl. In certain embodiments, the alkynyl group is substituted C₂₋₆ alkynyl.

The terms “alkylene,” “alkenylene,” “alkynylene,” or “heteroalkylene,” alone or as part of another substituent, mean, unless otherwise stated, a divalent radical derived from an alkyl, alkenyl, alkynyl, or heteroalkyl, respectively. The term “alkenylene,” by itself or as part of another substituent, means, unless otherwise stated, a divalent radical derived from an alkene. An alkylene, alkenylene, alkynylene, or heteroalkylene group may be described as, e.g., a C₁-C₆-membered alkylene, C₁-C₆-membered alkenylene, C₁-C₆-membered alkynylene, or C₁-C₆-membered heteroalkylene, wherein the term “membered” refers to the non-hydrogen atoms within the moiety. In the case of heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— may represent both —C(O)₂R′— and —R′C(O)₂—. Each instance of an alkylene, alkenylene, alkynylene, or heteroalkylene group may be independently optionally substituted, i.e., unsubstituted (an “unsubstituted alkylene”) or substituted (a “substituted heteroalkylene) with one or more substituents.

As used herein, “amino” refers to the radical —N(R^{A})(R^{B}), wherein each of R^{A} and R^{B} is independently hydrogen, alkyl, alkenyl, alkynyl, aryl, heteroaryl, cycloalkyl, or heterocyclyl.

As used herein, “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆-C₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). An aryl group may be described as, e.g., a C₆-C₁₀-membered aryl, wherein the term “membered” refers to the non-hydrogen ring atoms within the moiety. Aryl groups include phenyl, naphthyl, indenyl, and tetrahydronaphthyl. Each instance of an aryl group may be independently optionally substituted, i.e., unsubstituted (an “unsubstituted aryl”) or substituted (a “substituted aryl”) with one or more substituents. In certain embodiments, the aryl group is unsubstituted C₆-C₁₄ aryl. In certain embodiments, the aryl group is substituted C₆-C₁₄ aryl.

As used herein, the terms “arylene” and “heteroarylene,” alone or as part of another substituent, mean a divalent radical derived from an aryl and heteroaryl, respectively. Each instance of an arylene or heteroarylene may be independently optionally substituted, ie., unsubstituted (an “unsubstituted arylene”) or substituted (a “substituted heteroarylene”) with one or more substituents.

As used herein, the term “arylalkyl” refers to an aryl or heteroaryl group that is attached to another moiety via an alkylene linker. As used herein, the term “arylalkyl” refers to a group that may be substituted or unsubstituted. The term “arylalkyl” is also intended to refer to those compounds wherein one or more methylene groups in the alkyl chain of the arylalkyl group can be replaced by a heteroatom such as —O—, —Si— or —S—.

As used herein, “cycloalkyl” refers to a radical of a non-aromatic cyclic hydrocarbon group having from 3 to 10 ring carbon atoms (“C₃-C₁₀ cycloalkyl”) and zero heteroatoms in the non-aromatic ring system. In some embodiments, a cycloalkyl group has 3 to 9 ring carbon atoms (“C₃-C₉ cycloalkyl”). In some embodiments, a cycloalkyl group has 3 to 8 ring carbon atoms (“C₃-C₈ cycloalkyl”). In some embodiments, a cycloalkyl group has 3 to 7 ring carbon atoms (“C₃-C₇ cycloalkyl”). In some embodiments, a cycloalkyl group has 3 to 6 ring carbon atoms (“C₃-C₆ cycloalkyl”). In some embodiments, a cycloalkyl group has 5 to 7 ring carbon atoms (“C₅-C₇ cycloalkyl”). A cycloalkyl group may be described as, e.g., a C₄-C₇-membered cycloalkyl, wherein the term “membered” refers to the non-hydrogen ring atoms within the moiety. Exemplary C₃-C₆ cycloalkyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃-C₈ cycloalkyl groups include, without limitation, the aforementioned C₃-C₆ cycloalkyl groups as well as cycloheptyl (C₇), cycloheptenyl (C₇), cycloheptadienyl (C₇), and cycloheptatrienyl (C₇), bicyclo[2.1.1]hexanyl (C₆), bicyclo[3.1.1]heptanyl (C₇), cyclooctyl (C₈), cyclooctenyl (C₈), cubanyl (C₈), and the like. Exemplary C₃-C₁₀ cycloalkyl groups include, without limitation, the aforementioned C₃-C₈ cycloalkyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the cycloalkyl group is either monocyclic (“monocyclic cycloalkyl”) or contain a fused, bridged or spiro ring system such as a bicyclic system (“bicyclic cycloalkyl”) and can be saturated or can be partially unsaturated. “Cycloalkyl” also includes ring systems wherein the cycloalkyl ring, as defined above, is fused with one or more aryl groups wherein the point of attachment is on the cycloalkyl ring, and in such instances, the number of carbons continue to designate the number of carbons in the cycloalkyl ring system. Each instance of a cycloalkyl group may be independently optionally substituted, i.e., unsubstituted (an “unsubstituted cycloalkyl”) or substituted (a “substituted cycloalkyl”) with one or more substituents.

As used herein, the term “halo” or “halogen” refers to a fluorine, chlorine, bromine, or iodine radical (i.e., —F, —Cl, —Br, and —I).

As used herein, the term “heteroalkyl,” “heteroalkenyl,” and “heteroalkynyl,” refer to non-cyclic stable straight or branched chain alkyl, alkenyl, or alkynyl groups that include at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) O, N, P, S, and Si may be placed at any position of the heteroalkyl, heteroalkenyl, or heteroalkynyl groups. Exemplary heteroalkyl, heteroalkenyl, or heteroalkynyl groups include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —CH═CH—O—CH₃, —Si(CH₃)₃, —CH₂—CH═N—O—CH₃, —CH═CH—N(CH₃)—CH₃, —O—CH₃, and —O—CH₂—CH₃. Up to two or three heteroatoms may be consecutive, such as, for example, —CH₂—NH—OCH₃ and —CH₂—O—Si(CH₃)₃.

As used herein, the term “heteroaryl,” refers to an aromatic heterocycle that comprises 1, 2, 3 or 4 heteroatoms selected, independently of the others, from nitrogen, sulfur and oxygen. As used herein, the term “heteroaryl” refers to a group that may be substituted or unsubstituted. A heteroaryl may be fused to one or two rings, such as a cycloalkyl, an aryl, or a heteroaryl ring. The point of attachment of a heteroaryl to a molecule may be on the heteroaryl, cycloalkyl, heterocycloalkyl or aryl ring, and the heteroaryl group may be attached through carbon or a heteroatom. Examples of heteroaryl groups include imidazolyl, furyl, pyrrolyl, thienyl, thiazolyl, isoxazolyl, isothiazolyl, thiadiazolyl, oxadiazolyl, pyridinyl, pyrimidyl, pyrazinyl, pyridazinyl, quinolyl, isoquinolinyl, indazolyl, benzoxazolyl, benzisooxazolyl, benzofuryl, benzothiazolyl, indolizinyl, imidazopyridinyl, pyrazolyl, triazolyl, oxazolyl, tetrazolyl, benzimidazolyl, benzoisothiazolyl, benzothiadiazolyl, benzoxadiazolyl, indolyl, tetrahydroindolyl, azaindolyl, imidazopyridyl, quinazolinyl, purinyl, pyrrolo[2,3]pyrimidyl, pyrazolo[3,4]pyrimidyl or benzo(b)thienyl, each of which can be optionally substituted.

As used herein, “heterocyclyl” refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more cycloalkyl groups wherein the point of attachment is either on the cycloalkyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. A heterocyclyl group may be described as, e.g., a 3-7-membered heterocyclyl, wherein the term “membered” refers to the non-hydrogen ring atoms, i.e., carbon, nitrogen, oxygen, sulfur, boron, phosphorus, and silicon, within the moiety. Each instance of heterocyclyl may be independently optionally substituted, i.e., unsubstituted (an “unsubstituted heterocyclyl”) or substituted (a “substituted heterocyclyl”) with one or more substituents.

In some embodiments, a heterocyclyl group is a 5-10 membered non-aromatic ring system having ring carbon atoms and 1-4 ring heteroatoms, wherein each heteroatom is independently selected from nitrogen, oxygen, sulfur, boron, phosphorus, and silicon (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

As used herein, the term “hydroxy” refers to the radical —OH.

As used herein, the term “oxo” refers to the radical —C═O.

### c. Stereochemistry Considerations

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, N Y, 1962); and Wilen, Tables of Resolving Agents and Optical Resolutions p. 268 (E. L. Eliel, Ed., Univ. of Notre Dame Press, Notre Dame, Ind. 1972). The invention additionally encompasses compounds described herein as individual isomers substantially free of other isomers, and alternatively, as mixtures of various isomers.

As used herein, a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. In some embodiments, ‘substantially free’, refers to: (i) an aliquot of an “R” form compound that contains less than 2% “S” form; or (ii) an aliquot of an “S” form compound that contains less than 2% “R” form. The term “enantiomerically pure” or “pure enantiomer” denotes that the compound comprises more than 90% by weight, more than 91% by weight, more than 92% by weight, more than 93% by weight, more than 94% by weight, more than 95% by weight, more than 96% by weight, more than 97% by weight, more than 98% by weight, more than 99% by weight, more than 99.5% by weight, or more than 99.9% by weight, of the enantiomer. In certain embodiments, the weights are based upon total weight of all enantiomers or stereoisomers of the compound.

In the compositions provided herein, an enantiomerically pure compound can be present with other active or inactive ingredients. For example, a pharmaceutical composition comprising enantiomerically pure “R” form compound can comprise, for example, about 90% excipient and about 10% enantiomerically pure “R” form compound. In certain embodiments, the enantiomerically pure “R” form compound in such compositions can, for example, comprise, at least about 95% by weight “R” form compound and at most about 5% by weight “S” form compound, by total weight of the compound. For example, a pharmaceutical composition comprising enantiomerically pure “S” form compound can comprise, for example, about 90% excipient and about 10% enantiomerically pure “S” form compound. In certain embodiments, the enantiomerically pure “S” form compound in such compositions can, for example, comprise, at least about 95% by weight “S” form compound and at most about 5% by weight “R” form compound, by total weight of the compound. In certain embodiments, the active ingredient can be formulated with little or no excipient or carrier.

### 4. General

It is to be understood that the discussion set forth below in this “General” section can pertain to some, or to all, of the various embodiments of the invention(s) described herein.

### a. Methods of Producing Nanoparticles

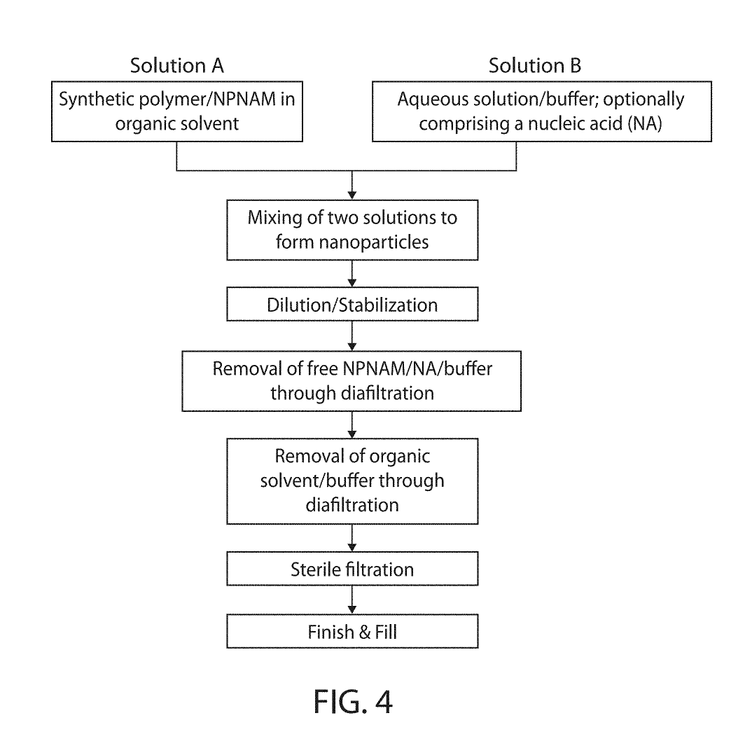

Described herein are methods for producing nanoparticles comprising a polymer (e.g., synthetic polymer, e.g. PLGA) that encapsulate/entrap nucleic acids and/or neutral or positively charged nucleic acid mimics (NPNAM). Applicant has found that the process described herein can be efficiently scaled compared with other formulation methods, and can also produce nanoparticles of higher quality in a more consistent manner. An example of the process described herein is depicted in the flow chart illustrated in FIG. 4. As shown in FIG. 4, two solutions are prepared and ultimately combined. The first solution can comprise a first solvent (e.g., water or buffer) and optionally comprise a nucleic acid (or mixture of nucleic acids) dissolved in the first solvent (e.g., water/buffer). The first solution may optionally comprise a small percentage of water miscible organic solvent. In the second solution, the NPNAM (or mixture of NPNAMs) and the polymer (e.g., synthetic polymer) are dissolved in a second solvent (e.g., an organic solvent, or an organic solvent that can optionally comprise a small percentage of water). As illustrated in FIG. 4, the first solution is mixed with the second solution to form nanoparticles (e.g. via nanoprecipitation) of the polymer (e.g., synthetic polymer) thereby encapsulating/entrapping the NPNAM(s), and if present the nucleic acid(s), within the nanoparticles so formed. As illustrated, after the initial step of mixing the first and second solutions, the mixture can be further diluted, typically with additional water or aqueous buffer to further stabilize the particles so formed. Once diluted to a desired concentration that permits formation of stable nanoparticles comprising the encapsulated/entrapped NPNAM(s), and nucleic acid(s) if present, removal of excess reagents can be performed if desired. For example, as illustrated in FIG. 4, excess (i.e. floating free in solution) NPNAM(s) and nucleic acids can be removed from the nanoparticles by diafiltration. The organic solvent and buffer can also be removed from the nanoparticles through, for example, diafiltration.

Again, with reference to FIG. 4, once the nanoparticles are properly free of contaminating excess reagents and buffer, they can optionally be sterilized. For example, by filtration through a filter of proper pore size so as to remove all bacteria and virus particles. Once sterile, the nanoparticles can optionally be transferred to a module for finish and fill of containers suitable for administration to patients.

### b. Features of Nanoparticles

The present disclosure features nanoparticles comprising a neutral or positively charged nucleic acid mimic (NPNAM) and a synthetic polymer and preparations thereof, as well as methods of making and using the same. In an embodiment, the NPNAM can comprise, for example, a peptide nucleic acid (PNA), morpholino, pyrrolidine-amide oligonucleotide mimic, morpholinoglycine oligonucleotide or methyl phosphonate. In some embodiments, the NPNAM is a peptide nucleic acid (PNA) oligomer. In some embodiments, the PNA oligomer is a tail-clamp peptide nucleic acid (tcPNA).

In some embodiments, the NPNAM is a PNA oligomer comprising a structure of Formula (I):

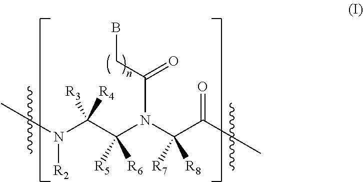

wherein B, R₂, R₃, R₄, R₅, R₆, R₇, R₈; n is an integer from 0 to 3, inclusive; and “ [symbol] ” is previously defined.

In some embodiments, B is a naturally occurring nucleobase (e.g., adenine, cytosine, guanine, thymine, or uracil). In some embodiments, B is a non-naturally occurring nucleobase, e.g., pseudoisocytosine (i.e., j). In some embodiments, B is selected from adenine, cytosine, guanine, thymine, uracil, and pseudoisocytosine. In some embodiments, B is selected from adenine, cytosine, guanine, thymine, uracil, pseudoisocytosine, 2-thiopseudoisocytosine, hypoxanthine, 2-aminoadenine (a.k.a. 2,6-diaminopurine), 2-thiouracil, 2-thiothymine, 7-deazaguanine, 7-deazaadenine and 7-deaza-2,6-diaminopurine.

In some embodiments, R₃ and/or R₄ is heteroalkyl (e.g., a polyethylene glycol, e.g., a C₂-C₃₀ polyethylene glycol). In some embodiments, R₃ is heteroalkyl (e.g., a polyethylene glycol, e.g., a C₂-C₃₀ polyethylene glycol) and R₄ is hydrogen. In some embodiments, R₄ is a polyethylene glycol (e.g., a C₂-C₃₀ polyethylene glycol) and R₃ is hydrogen. In some embodiments, R₃ is (—CH₂—(OCH₂CH₂)_{q}—OH), wherein q is an integer from 1-3, inclusive. In some embodiments, R₄ is (—CH₂—(OCH₂CH₂)_{q}—OH), wherein q is an integer from 1-3, inclusive. In some embodiments, each of R₃ and R₄ is independently (—CH₂—(OCH₂CH₂)_{q}—OH), wherein q is an integer from 1-3, inclusive.

In some embodiments, each of R₂, R₃, R₄, R₅, R₆, R₇ and R₈ is independently hydrogen or deuterium. In some embodiments, each of R₂, R₃, and R₄ is independently hydrogen. In some embodiments, each of R₃ and R₄ is independently hydrogen. In some embodiments, R₂ is hydrogen or methyl. In some embodiments, R₃ is hydrogen. In some embodiments, R₄ is hydrogen.

In some embodiments, B is selected from adenine, cytosine, guanine, thymine, uracil, pseudoisocytosine, 2-thiopseudoisocytosine, hypoxanthine, 2-aminoadenine (a.k.a. 2,6-diaminopurine), 2-thiouracil, 2-thiothymine, 7-deazaguanine, 7-deazaadenine and 7-deaza-2,6-diaminopurine; R₃ is a polyethylene glycol (e.g., a C₂-C₃₀ polyethylene glycol such as: —CH₂—(OCH₂CH₂)_{q}—OH), wherein q is an integer from 1-3, inclusive); each of R₂, R₄, R₅, R₆, R₇ and R₈ is hydrogen and n is 1, 2 or 3.

In some embodiments, B is selected from adenine, cytosine, guanine, thymine, uracil, pseudoisocytosine, 2-thiopseudoisocytosine, hypoxanthine, 2-aminoadenine (a.k.a. 2,6-diaminopurine), 2-thiouracil, 2-thiothymine, 7-deazaguanine, 7-deazaadenine and 7-deaza-2,6-diaminopurine; R₄ is a polyethylene glycol (e.g., a C₂-C₃₀ polyethylene glycol such as: (—CH₂—(OCH₂CH₂)_{q}—OH), wherein q is an integer from 1-3, inclusive); each of R₂, R₃, R₅, R₆, R₇ and R₈ is hydrogen and n is 1, 2 or 3.

In some embodiments, n is 1. In some embodiments, n is 2. In some embodiments, n is 3.

In some embodiments, the NPNAM is a PNA oligomer comprising greater than 2, 3, 4, 5, 6, 7, 8, 9, 10, 15, 20, 25, 30, 35, 40, 45, or 50 residues. In some embodiments, the NPNAM is a PNA oligomer comprising between about 5 to 50 residues, e.g., between about 6 and 45, 8 and 40, 12 and 38, 15 and 36, and 18 and 24 residues.

In some embodiments, the NPNAM is a PNA oligomer comprising a structure of Formula (I-a):

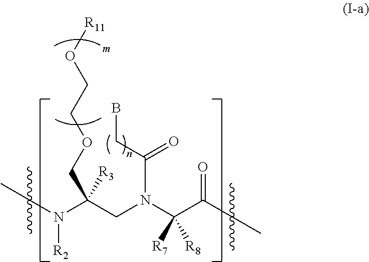

wherein B, R₂, R₃, R₇, R₈, “ [symbol] ” is oreviously defined, R₁₁ is hydrogen or C₁-C₄ alkyl, m is an integer from 1 to 3 (inclusive) and n is 1 to 3 (inclusive).

In some embodiments, B is a nucleobase and each of R₂, R₃, R₇ and R₈ is independently hydrogen, deuterium or C₁-C₄ alkyl.

In some embodiments, B is a naturally occurring nucleobase (e.g., adenine, cytosine, guanine, thymine, or uracil). In some embodiments, B is a non-naturally occurring nucleobase, e.g., pseudoisocytosine (i.e., j). In some embodiments, B is selected from is selected from adenine, cytosine, guanine, thymine, uracil, and pseudoisocytosine. In some embodiments, B is selected from adenine, cytosine, guanine, thymine, uracil, pseudoisocytosine, 2-thiopseudoisocytosine, hypoxanthine, 2-aminoadenine (a.k.a. 2,6-diaminopurine), 2-thiouracil, 2-thiothymine, 7-deazaguanine, 7-deazaadenine and 7-deaza-2,6-diaminopurine.

In some embodiments, the NPNAM is a PNA comprising a structure of Formula (I-b):

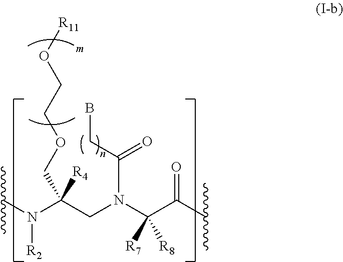

wherein B, R₂, R₄, R₇, R₈, n and “ [symbol] ” are previously defined, R₁₁ is alkyl and m is an integer from 1 to 3, inclusive.

In some embodiments, B is a nucleobase; each of R₂, R₄, R₇ and R₈ is independently hydrogen, D or C₁-C₄ alkyl.

In some embodiments, B is a naturally occurring nucleobase (e.g., adenine, cytosine, guanine, thymine, or uracil). In some embodiments, B is a non-naturally occurring nucleobase, e.g., pseudoisocytosine (i.e., j). In some embodiments, B is selected from is selected from adenine, cytosine, guanine, thymine, uracil, and pseudoisocytosine. In some embodiments, B is selected from adenine, cytosine, guanine, thymine, uracil, pseudoisocytosine, 2-thiopseudoisocytosine, hypoxanthine, 2-aminoadenine (a.k.a. 2,6-diaminopurine), 2-thiouracil, 2-thiothymine, 7-deazaguanine, 7-deazaadenine and 7-deaza-2,6-diaminopurine.

In some embodiments, the NPNAM comprising a structure of Formula (I) is a tail-clamp PNA (tcPNA). In some embodiments, the NPNAM comprising a structure of Formula (I-a) is a tail-clamp PNA (tcPNA). In some embodiments, the NPNAM comprising a structure of Formula (I-b) is a tail-clamp PNA (tcPNA).

In some embodiments, the NPNAM comprises a PNA having the sequence of PNA-1 or PNA-2. In an embodiment, the NPNAM comprises a PNA having the sequence of PNA-1. In an embodiment, the NPNAM comprises a PNA having the sequence of PNA-2.

The amount of a NPNAM encapsulated and/or entrapped within the nanoparticle may vary depending on the identity of the NPNAM or plurality of NPNAMs. For example, the amount of NPNAM may be between 0.05% and 40% by weight of NPNAMs to the total weight of the nanoparticle. In some embodiments, the amount of NPNAM in the nanoparticle is greater than about 0.05%, e.g., greater than about 0.1%, 0.5%, 1%, 2%, 3%, 4%, 5%, 6%, 7%, 8%, 9%, 10%, 12.5%, 15%, 20%, 25%, 30%, 35%, or 40% by weight of NPNAM to the total weight of the nanoparticle. In some embodiment, the amount of NPNAM in the nanoparticle is between 0.5% and 20% by weight of NPNAMs to the total weight of the nanoparticle, or between 1% and 10% by weight of NPNAM to the total weight of the nanoparticle, or between 2% to 5% by weight of NPNAM to the total weight of the nanoparticle.

A nanoparticle described herein may comprise a single type of NPNAM (e.g., a single type of PNA oligomer, or a PNA oligomer of a single sequence), or may comprise multiple types of NPNAMs. In some embodiments, the nanoparticle comprises a single type of NPNAM. In some embodiments, the nanoparticles comprise a plurality of NPNAMs (e.g., a plurality of PNAs).

In addition to a NPNAM, the nanoparticles of the present disclosure comprise a polymer, e.g., a synthetic polymer. The synthetic polymer may be linear or branched. In some embodiments, the synthetic polymer comprises a co-polymer or conjugate (e.g. PLGA-PEG or PLA-PEG). In some embodiments, the synthetic polymer is a mixture or blend of polymers. In some embodiments, the synthetic polymer is degradable (e.g., biodegradable). In some embodiments, the synthetic polymer is a mixture or blend of polymers such as a mixture of PLGA and PEG, PLA and PEG, or PLGA, PLA and PEG. Exemplary synthetic polymers include polylactic acid (PLA), polyglycolic acid, (PGA), poly(lactic-co-glycolic acid) (PLGA), poly(4-hydroxy-L-proline ester), a polyester, a polyanhydride, a poly(ortho)ester, a polyurethane, a poly(butic acid), poly(valeric acid), poly(caprolactone), a poly(hydroxyalkanoate), a poly(lactide-co-caprolactone), a poly(amine-co-ester) polymer, or a combination of any two or more of the foregoing, including, blends, co-polymers or conjugates with, for example, a PEG polymer. In some embodiments, the synthetic polymer is PLGA. In some embodiments, the synthetic polymer is PLGA. In some embodiments, the synthetic polymer is PLGA-PEG.

A synthetic polymer may be any size. For example, a synthetic polymer may range in size from 500 Da to about 1,000,000 Da (e.g., from about 1,000 Da to about 500,000 Da, from about 1,000 Da to about 100,000 Da, or from about 5,000 Da to about 50,000 Da).

In some embodiments, the synthetic polymer comprises a structure of Formula (II):

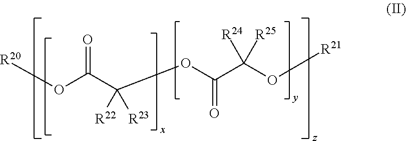

wherein each of R²⁰ and R²¹ is independently hydrogen or alkyl; each of R²², R²³, R²⁴, and R²⁵ is independently hydrogen, alkyl, alkenyl, alkynyl, heteroalkyl, cycloalkyl, heterocyclyl, aryl, or heteroaryl, wherein each of alkyl, alkenyl, alkynyl, heteroalkyl, cycloalkyl, heterocyclyl, aryl, and heteroaryl is optionally substituted with one or more R²⁶; R²⁶ is hydrogen or alkyl; each of x and y is an integer between 0 and 100 (inclusive), wherein each of x and y cannot simultaneously be 0; and z is an integer between 1 and 10,000 (inclusive).

In some embodiments, each of R²⁰ and R²¹ is independently hydrogen. In some embodiments, each of R²⁰ and R²¹ is independently alkyl.

In some embodiments, one of R²² and R²³ is hydrogen and the other of R²² and R²³ is alkyl (e.g., methyl). In some embodiments, each of R²⁴ and R²⁵ is independently hydrogen.

In some embodiments, x is an integer greater than 0 and y is an integer greater than 0. In some embodiments, y is 0. In some embodiments, y is an integer between 1 and 50 (inclusive), between 1 and 25 (inclusive), between 1 and 10 (inclusive), or between 1 and 5 (inclusive). In some embodiments, y is 1. In some embodiments, x is 0. In some embodiments, x is an integer between 1 and 50 (inclusive), between 1 and 25 (inclusive), between 1 and 10 (inclusive), or between 1 and 5 (inclusive). In some embodiments, x is 1.

In some embodiments, z is an integer between 1 and 1,000 (inclusive), between 1 and 5,000 (inclusive), between 1 and 2,500 (inclusive), between 1 and 1,000 (inclusive), between 1 and 750 (inclusive), between 1 and 500 (inclusive), between 1 and 250 (inclusive), between 1 and 100 (inclusive), or between 1 and 50 (inclusive).

In some embodiments, the synthetic polymer having a structure of Formula (II) is selected from PLGA, PGA, and PLA. In some embodiments, the synthetic polymer having a structure of Formula (II) is PLGA. In some embodiments, the synthetic polymer having a structure of Formula (II) is PGA. In some embodiments, the synthetic polymer having a structure of Formula (II) is PLA.

In some embodiments, the synthetic polymer may further comprise a polyethylene glycol moiety. For example, the synthetic polymer comprising a structure of Formula (II) may further comprise a PEG moiety. Exemplary synthetic polymers include mPEG-PLA or mPEG-PLGA.

In an embodiment, nanoparticle comprises a single type of synthetic polymer. In an embodiment, the nanoparticle comprises a plurality of synthetic polymers. For example, a nanoparticle of the present disclosure may comprise PLGA, or may comprise PLGA and a second synthetic polymer. An example would include a nanoparticle containing both PLGA and mPEG-PLGA.

The amount of a synthetic polymer encapsulated and/or entrapped within the nanoparticle may vary depending on the identity of the synthetic polymer or plurality of synthetic polymer. For example, the amount of a synthetic polymer may be between 0.05% and 90% by weight of synthetic polymers to the total weight of the nanoparticle. In some embodiments, the amount of a synthetic polymer in the nanoparticle is greater than about 0.05%, e.g., greater than about 0.1%, 0.5%, 1%, 2%, 3%, 4%, 5%, 6%, 7%, 8%, 9%, 10%, 12.5%, 15%, 20%, 25%, 30%, 35%, 40%, 45%, 55%, 60%, 65%, 70%, 75%, 80%, 85% or 90% by weight of synthetic polymers to the total weight of the nanoparticle. In some embodiment, the amount of a synthetic polymer in the nanoparticle is between 0.5% and 20% by weight of synthetic polymers to the total weight of the nanoparticle, or between 1% and 10% by weight of a synthetic polymer to the total weight of the nanoparticle, or between 2% to 5% by weight of a synthetic polymer to the total weight of the nanoparticle.

A nanoparticle may further comprise a load component. A load component may be any additional biological component (e.g., a polymeric biological component), for example, a nucleic acid or polypeptide. In some embodiments, the load component is a nucleic acid. A nucleic acid may be double stranded or single stranded, for example, an oligonucleotide, a single stranded DNA, a single stranded RNA, a double stranded DNA, or a double stranded RNA. In some embodiments, the load component is a nucleic acid (e.g., DNA) between 5 and 250 nucleotides in length (e.g., between 10 and 200 nucleotides in length, or 20 and 100 nucleotides in length).

In some embodiments, the load component is a nucleic acid and comprises one phosphorothioate linkage at a terminus. In some embodiments, the load component comprises more than one phosphorothioate linkages at a terminus, for example, at each of its 3′ and 5′ termini. The nucleic acid may comprise 2, 3, 4, 5, 6, 7, 8, 9, 10 or more phosphorothioate linkages at a terminus. In some embodiments, the nucleic acid comprises 1, 2 or 3 phosphorothioate linkages at each of its 3′ and 5′ termini, wherein the number of phosphorothioate linkages can be the same or different at each of the 3′ and 5′ termini.

The load component may comprise a nucleic acid that is an antisense agent. A load component may comprise a nucleic acid having a sequence which is the same or the complement of a sequence to which the NPNAM, e.g., a clamp system, e.g., a tail clamp system, e.g., a PNA oligomer comprising a structure of Formula (I) as described herein. In some embodiments, the load component has Watson Crick homology and optionally Hoogsteen homology to a sequence. A load component may comprise a nucleic acid having a sequence which is the same or the complement of a sequence to which the NPNAM, e.g., a clamp system, e.g., a tail clamp system, e.g., a PNA oligomer comprising a structure of Formula (I) as described herein, has Hoogsteen homology. A load component may comprise a nucleic acid having a sequence that is the same or the complement of a sequence that is within 1,000, 500, or 200 base pairs of a sequence to which the NPNAM, e.g., a clamp system, e.g., a tail clamp system, e.g., a PNA oligomer comprising a structure of Formula (I) as described herein, has Watson Crick homology. A load component may comprise a nucleic acid having a sequence which is the same or the complement of a sequence that is within 1,000, 500, or 200 base pairs of a sequence to which the NPNAM, e.g., a clamp system, e.g., a tail clamp system, e.g., a PNA oligomer comprising a structure of Formula (I) as described herein, has Hoogsteen homology.

In some embodiments, a nanoparticle may further comprise polyethylene glycol (PEG) conjugated to a synthetic polymer. A polyethylene glycol may be any size, for example, between 2 PEG subunits and 5,000 PEG subunits. Inclusion of polymer-PEG in the nanoparticles can improve the long-term stability of cryopreserved nanoparticles.

A nanoparticle described herein may have a diameter between 5 and 500 nm, e.g., between 25 and 400 nm, 35 and 350 nm, 40 and 250 nm, 50 and 200 nm, 150 and 250 nm, 175 and 325 nm, 180 and 250 nm, 250 nm and 310 nm and 30 and 100 nm. In some embodiments, the nanoparticle has a diameter between 20 and 100 nm, between 20 and 80 nm, and between 20 and 60 nm. In some embodiments, the nanoparticle has a diameter between 175 and 325 nm. In some embodiments, the nanoparticle has a diameter between 250 nm and 310 nm.

In some embodiments, a nanoparticle described herein has a neutral to negative surface charge of less than −100 mv, for example, less than −90 mv, −80 mv, −70 mv, −60 mv, −50 mv, −40 mv, −30 mv, and −20 mv. In some embodiments, a nanoparticle described herein has a neutral to negative surface charge of between −100 my and 100 mv, between −75 my to 0, −30 my to −5 my or between −50 my and −10 mv.

In the presence of a target sequence, a nanoparticle described herein may behave in a certain manner. For example, in some embodiments, a nanoparticle, when contacted with a target nucleic acid, allows binding of its component NPNAM to a target nucleic acid sequence, e.g., as evaluated by UV melting temperature in hybridization experiments (e.g., at 260 nm), thermodynamic analysis, or surface plasmon resonance. In some embodiments, a nanoparticle, when contacted with a target nucleic acid, decreases the Tm of a target nucleic acid sequence, e.g., as evaluated by UV melting temperature in hybridization experiments (e.g., at 260 nm), thermodynamic analysis, or surface plasmon resonance. In some embodiments, a nanoparticle, when contacted with a target nucleic acid, promotes melting or dissociation of the strands of a target nucleic acid sequence, e.g., as evaluated by strand invasion assay. In some embodiments, a nanoparticle, when contacted with a target nucleic acid, allows its component NPNAM to cleave a target nucleic acid sequence. In some embodiments, a nanoparticle, when contacted with a target nucleic acid, allows its component NPNAM and nucleic acid to edit a target nucleic acid sequence, e.g., as evaluated by NGS or ddPCR.

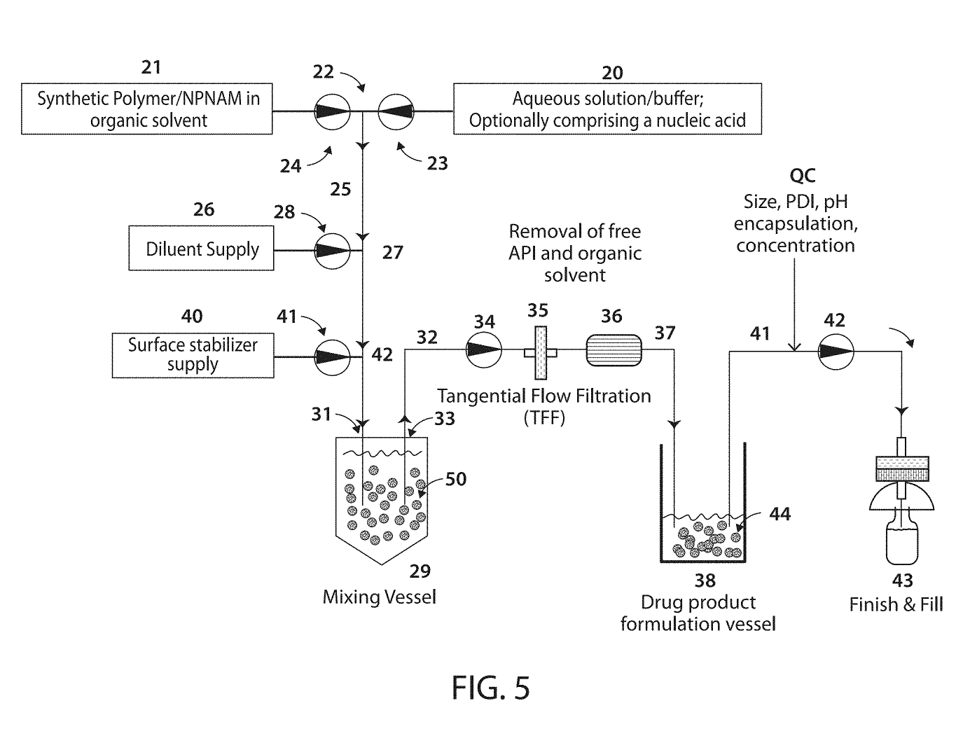

In one aspect, the present disclosure features a nanoparticle prepared by a method described herein. For example, the nanoparticle is prepared by the nanoprecipitation method illustrated in, e.g., FIG. 4 or FIG. 5.

### c. Preparations of Nanoparticles

The present disclosure features a preparation comprising a plurality of nanoparticles, wherein each nanoparticle of the plurality comprises a neutral or positively charged nucleic acid mimic (NPNAM(s)) and a synthetic polymer or mixture of synthetic polymers (including without limitation any form of polymer conjugate, co-polymer, block polymer, polymer mixture and/or polymer blend). In some embodiments, the NPNAM is a NPNAM as described herein, e.g., a PNA oligomer, e.g., a PNA oligomer comprising a structure of Formula (I). In some embodiments, the synthetic polymer is a synthetic polymer described herein, e.g., selected from include polylactic acid (PLA), polyglycolic acid, (PGA), poly(lactic-co-glycolic acid) (PLGA), poly(4-hydroxy-L-proline ester), a polyester, a polyanhydride, a poly(ortho)ester, a polyurethane, a poly(butic acid), poly(valeric acid), poly(caprolactone), a poly(hydroxyalkanoate), a poly(lactide-co-caprolactone), a poly(amine-co-ester) polymer, or a combination of any two or more of the foregoing, including copolymers (e.g. PEG copolymers), polymer conjugates (e.g. PEG conjugates), block polymers (e.g. PEG block polymers), polymer blends or polymer mixtures. In some embodiments, the synthetic polymer comprises the structure of Formula (II).

The preparation comprising a plurality of nanoparticles may further comprise one of the following properties: (i) the amount of neutral or positively charged nucleic acid mimic(s) encapsulated and/or entrapped within each nanoparticle of the plurality is greater than or equal to 0.05% by weight of NPNAM(s) to the total weight of the nanoparticle(s); (ii) at least 5% of the nanoparticles of the preparation have a diameter of between 5 and 500 nm; (iii) at least 5% of the nanoparticles of the preparation have a neutral to negative surface charge of less than −100 mv, (iv) the preparation contains less than 0.05% by weight of free NPNAM, free synthetic polymer, or a free load component; or (v) the preparation contains less than 0.05% by weight of empty nanoparticles.

In some embodiments, the preparation comprises two of properties (i)-(v). In some embodiments, the preparation comprises three of properties (i)-(v). In some embodiments, the preparation comprises four of properties (i)-(v). In some embodiments, the preparation comprises all of properties (i)-(v). In some embodiments, the preparation comprises property (i). In some embodiments, the preparation comprises property (ii). In some embodiments, the preparation comprises property (iii). In some embodiments, the preparation comprises property (iv). In some embodiments, the preparation comprises property (v).

The preparation comprising a plurality of nanoparticles described herein may comprise an amount of NPNAM(s) greater than or equal to about 0.05% by weight of NPNAM(s) to the total weight of the nanoparticle(s), e.g., greater than about 0.1%, 0.5%, 1%, 2%, 3%, 4%, 5%, 6%, 7%, 8%, 9%, 10%, 12.5%, 15%, 20%, 25%, 30%, 35%, or 40% by weight of NPNAM(s) to the total weight of the nanoparticle(s). In some embodiments, the preparation comprising a plurality of nanoparticles comprises an amount of NPNAM(s) between about 0.05% to about 40% by weight NPNAM(s) to the total weight of the nanoparticle(s). In some embodiments, the amount of NPNAM(s) in the plurality of nanoparticles in the preparation is between 0.5% and 20% by weight, or between 1% and 10% by weight, or between 2% to 5.

The preparation comprising a plurality of nanoparticles described herein may have an average diameter between 5 and 500 nm, e.g., between 10 and 400 nm, 20 and 300 nm, 25 and 250 nm, 30 and 200 nm, and 30 and 100 nm. In some embodiments, the preparation comprising a plurality of nanoparticles has an average diameter between 20 and 100 nm, between 20 and 80 nm, and between 20 and 60 nm.

In some embodiments, at least 5% (e.g., at least 10%, at least 15%, at least 20%, at least 25%, at least 30%, at least 40%, at least 50%, at least 60%, at least 70%, at least 80%, at least 90%, at least 95%, or at least 99%) of the nanoparticles of the preparation have a diameter between 5 and 500 nm. The nanoparticles of the preparation may range in diameter from about 10 to 400 nm, 20 to 300 nm, 25 to 250 nm, 30 to 200 nm, and 30 to 100 nm. In some embodiments, the nanoparticles of the preparation may range in diameter from about 20 to 100 nm, 20 to 80 nm, and between 20 to 60 nm.

In some embodiments, the preparation comprising a plurality of nanoparticles described herein has an average neutral to negative surface charge of less than −100 mv, for example, less than −90 mv, −80 mv, −70 mv, −60 mv, −50 mv, −40 mv, −30 mv, and −20 myv. In some embodiments, the preparation comprising a plurality of nanoparticles has a neutral to negative surface charge of between −100 my and 100 mv, between −75 my to 0, or between −50 my and −10 mv.

In some embodiments, at least 5% (e.g., at least 10%, at least 15%, at least 20%, at least 25%, at least 30%, at least 40%, at least 50%, at least 60%, at least 70%, at least 80%, at least 90%, at least 95%, or at least 99%) of the nanoparticles of the preparation have an average neutral to negative surface charge of less than −100 mv. In some embodiments, the preparation comprising a plurality of nanoparticles has an average neutral to negative surface charge of between −100 my and 100 mv, between −75 my to 0, between −50 my and −10 my or between −35 my and −2 mv.

In some embodiments, at least 0.05% (e.g., at least about 0.1%, 0.5%, 1%, 2%, 3%, 4%, 5%, 6%, 7%, 8%, 9%, 10%, 12.5%, 15%, 20%, 25%, 30%, 35%, or 40%) of the nanoparticles in the preparation contain less than 0.05% by weight of free NPNAM, free synthetic polymer, or a free load component. In some embodiments, at least 0.05% (e.g., at least about 0.1%, 0.5%, 1%, 2%, 3%, 4%, 5%, 6%, 7%, 8%, 9%, 10%, 12.5%, 15%, 20%, 25%, 30%, 35%, or 40%) of the nanoparticles in the preparation contain less than 0.05% by weight of free NPNAM. In some embodiments, at least 0.05% (e.g., at least about 0.1%, 0.5%, 1%, 2%, 3%, 4%, 5%, 6%, 7%, 8%, 9%, 10%, 12.5%, 15%, 20%, 25%, 30%, 35%, or 40%) of the nanoparticles in the preparation contain less than 0.05% by weight free synthetic polymer. In some embodiments, at least 0.05% (e.g., at least about 0.1%, 0.5%, 1%, 2%, 3%, 4%, 5%, 6%, 7%, 8%, 9%, 10%, 12.5%, 15%, 20%, 25%, 30%, 35%, or 40%) of the nanoparticles in the preparation contain less than 0.05% by weight of a free load component.

In some embodiments, at least 5% (e.g., at least 10%, at least 15%, at least 20%, at least 25%, at least 30%, at least 40%, at least 50%, at least 60%, at least 70%, at least 80%, at least 90%, at least 95%, or at least 99%) of the nanoparticles in the plurality comprise a second type of NPNAM. In some embodiments, each nanoparticle of the plurality comprises a second type of NPNAM. In some embodiments, at least 5% (e.g., at least 10%, at least 15%, at least 20%, at least 25%, at least 30%, at least 40%, at least 50%, at least 60%, at least 70%, at least 80%, at least 90%, at least 95%, or at least 99%) of the nanoparticles in the plurality comprise a plurality of NPNAMs. In some embodiments, each nanoparticle of the plurality comprises a plurality of NPNAMs.

In some embodiments, at least 5% (e.g., at least 10%, at least 15%, at least 20%, at least 25%, at least 30%, at least 40%, at least 50%, at least 60%, at least 70%, at least 80%, at least 90%, at least 95%, or at least 99%) of the nanoparticles in the plurality comprise a second type of synthetic polymer. In some embodiments, each nanoparticle of the plurality comprises a second type of synthetic polymer. In some embodiments, at least 5% (e.g., at least 10%, at least 15%, at least 20%, at least 25%, at least 30%, at least 40%, at least 50%, at least 60%, at least 70%, at least 80%, at least 90%, at least 95%, or at least 99%) of the nanoparticles in the plurality comprise a plurality of synthetic polymers. In some embodiments, each nanoparticle of the plurality comprises a plurality of synthetic polymers.

The preparation comprising a plurality of nanoparticles may further comprise nanoparticles comprising a load component, e.g., a load component described herein. A load component may be any additional biological component (e.g., a polymeric biological component), for example, a nucleic acid or polypeptide. In some embodiments, the load component is a nucleic acid. A nucleic acid may be double stranded or single stranded, for example, an oligonucleotide, a single stranded DNA, a single stranded RNA, a double stranded DNA, or a double stranded RNA. In some embodiments, the load component is a nucleic acid (e.g., DNA) between 5 and 250 nucleotides in length (e.g., between 10 and 200 nucleotides in length, or 20 and 100 nucleotides in length). In some embodiments, at least 5% (e.g., at least 10%, at least 15%, at least 20%, at least 25%, at least 30%, at least 40%, at least 50%, at least 60%, at least 70%, at least 80%, at least 90%, at least 95%, or at least 99%) of the nanoparticles in the plurality comprise a load component.

The preparation comprising a plurality of nanoparticles may further comprise nanoparticles comprising a polyethylene glycol (PEG)-polymer conjugate, PEG-copolymer or PEG block polymer, e.g., a PEG described herein. A polyethylene glycol may be any size, for example, between 2 PEG subunits and 5,000 subunits. In some embodiments, at least 5% (e.g., at least 10%, at least 15%, at least 20%, at least 25%, at least 30%, at least 40%, at least 50%, at least 60%, at least 70%, at least 80%, at least 90%, at least 95%, or at least 99%) of the nanoparticles in the plurality comprise a PEG.

In one aspect, a preparation comprising a plurality of nanoparticles disclosed herein is made by a method described herein. For example, the preparation may be prepared using a method depicted in any one of FIG. 4 or FIG. 5.

In some embodiments, the preparation comprising a plurality of nanoparticles is a pharmaceutically acceptable preparation.

The preparation described herein may be disposed in a delivery device, for example, for ease of use. Exemplary delivery devices include a cannula, a syringe, a depot, a pump, or a tube. In some embodiments, the preparation described herein is disposed in a storage device, such as a vial.

### d. Apparatus

The present disclosure also described an apparatus suitable for formation of nanoparticles comprising a synthetic polymer and a NPNAM, e.g., as illustrated in FIG. 5. As shown in FIG. 5, the apparatus may comprise a first solvent supply 20 and a second solvent supply 21. The first solvent supply 20 can comprise water, an aqueous solution or aqueous buffer. In some embodiments, the first solvent supply further comprises up to at least 60% by volume of at least one water miscible organic solvent, e.g., up to at least about 0.05%, 0.1%, 0.5%%, 1%, 2%, 3%, 4%, 5%, 10%, 15%, 20%, 25%, 30%, 35%, 40%, 45%, 50%, 55% or 60% by volume of at least one organic solvent (e.g., a water miscible organic solvent). In an embodiment, the first solvent supply comprises between about 0.05% and 60% by volume organic solvent, e.g., between about 0.05% and 50%, about 0.05% and 40%, or about 5% and 20% by volume organic solvent (e.g., a water miscible organic solvent).

The second solvent supply 21 comprises at least one neutral or positively charged nucleic acid mimic (NPNAM) and at least one water miscible organic solvent. In some embodiments, the second solvent supply further comprises up to at least 60% by volume of water, e.g., up to at least about 0.05%, 0.1%, 0.5%%, 1%, 2%, 3%, 4%, 5%, 10%, 15%, 20%, 25%, 30%, 35%, 40%, 45%, 50%, 55% or 60% water. In an embodiment, the second solvent supply comprises between about 0.05% and 60% water, e.g., between about 0.05% and 50%, about 0.05% and 40%, about 5% and 20% water. In an embodiment, the second solvent supply comprises up to 40% by volume of water.

The apparatus further comprises at least one junction 22 to which the first solvent supply and second solvent supply are in fluid connection. This junction can permit efficient mixing of the first and second solvents (and their associated contents) as they are forced into said junction and as they exit the junction into a post-junction conduit 25 that contains the post-junction fluid stream. In some cases, junction 22 is referred to as a ‘tee’ mixer or ‘tee’ or just ‘T” junction because it can be made to resemble the letter “T”. However, there in no requirement that the geometry of the mixer be a made in a T-shape. For example, a Y-shape or other three way junction would also suffice.

A supply may comprise any physical mode that can affect the supply of the first and second solvents to the junction (or the diluent supply to the post-junction conduit as described below). For example, each supply can be a reservoir wherein the components of each solvent to be supplied to the junction are first mixed in appropriate concentrations and then delivered to the appropriate junction. Alternatively, the ‘supply’ can be a conduit into which more than one solution is combined (typically at another junction) in a ratio suitable to produce the supply of reagent(s) in the proper ratios and concentrations needed to feed said junction. Simply stated, so long as the relevant junction is properly supplied, it is not relevant how such supply is created.

In order to achieve efficient mixing in the post-junction fluid stream, the apparatus can optionally comprise one or more pumps in fluid connection with junction and one or more of the supplies. For example, the apparatus as illustrated in FIG. 5 comprises a pump 23 that is in fluid connection with the first solvent supply and the junction and a pump 24 that is in fluid connection with the second solvent supply. If pumps are not used, pressurized chambers or even gravity can be used to deliver the first solvent supply and the second solvent supply to the junction so long as mixing in the post-junction fluid stream is efficient enough to induce nanoprecipitation of the synthetic polymer and encapsulation/entrapment of the NPNAM(s) and optionally any nucleic acid(s) that might be added to (or present in) the first solvent supply.

As illustrated in FIG. 5, the apparatus can optionally further comprise at least one additional diluent supply 26 that is in fluid connection with the post-junction conduit, preferably at a second junction 27 (as illustrated) but optionally at the junction 22. The fluid connection between the diluent supply and the junction (27 or 22) may optionally comprise a pump 28 that is suitable to pump the contents of the diluent supply through said junction and into the post-junction conduit. Again, a pump is optional as gravity or a pressurized supply may suffice to provide sufficient flow into the post-junction conduit. The diluent supply 26 is generally added to stabilize the newly formed nanoparticles.

As illustrated in FIG. 5, the apparatus optionally further comprises a mixing vessel 29 positioned to capture the post-junction fluid stream that exits the post-junction conduit. Said vessel can be open or closed. If closed, it can comprise at least one input port 31 in fluid connection with the post-junction conduit and positioned to receive the post-junction fluid stream.

As illustrated in FIG. 5, the apparatus optionally further comprises a surface stabilizer supply 40 in fluid connection with the post-junction conduit at yet another junction 42 to thereby introduce a solution comprising surface stabilizer (e.g. sucrose, trehalose or cyclodextrin) to the post-junction conduit. The surface stabilizer supply may be actively pumped into the post-junction conduit using a pump 41. Again, a pump is optional as gravity or a pressurized supply may suffice to provide sufficient flow of the surface stabilizer supply into the post-junction conduit.

It is to be understood that this is not the only possible configuration for the surface stabilizer supply. For example, the apparatus optionally further comprises an input port fluidly connected directly to the mixing vessel and positioned and suitable to introduce the surface stabilizer supply (e.g. sucrose, trehalose or cyclodextrin) directly into the mixing vessel. Other configurations are within the scope of this disclosure so long as they permit mixing of the surface stabilizer supply with nanoparticles so formed and that comprise entrapped/encapsulated NPNAM(s) and optionally one or more load components.

As illustrated in FIG. 5, the apparatus also optionally includes an exit port 33, structured and positioned to permit fluid contents of the mixing vessel to exit the mixing vessel and flow into a post-mixing vessel conduit 32. The apparatus may also optionally comprise yet another pump 34 structured and positioned to permit the pumping of a solution comprising nanoparticles 50 that has accumulated in said mixing vessel 29 into the post-mixing vessel conduit 32 to thereby produce a post-mixing vessel fluid stream contained by said post-mixing vessel conduit 32.

As illustrated in FIG. 5, the apparatus also optionally includes tangential flow filtration (TFF) devices 35 and 36 in fluid communication with the post-mixing vessel conduit 32. As illustrated, the post-mixing vessel fluid stream passes through said tangential flow filtration devices 35 and 36 to thereby permit removal of organic solvent, buffer, excess NPNAM(s), load component(s) (e.g. nucleic acid(s)) and/or any small molecule contaminates that might be undesired or detrimental to a pharmaceutical product. For example, tangential flow filtration devices 35 and 36 can be affixed in-line in the post-mixing vessel conduit 32. When functioning, contaminates can be shunted away from the tangential flow filtration devices 35 and 36 and post-mixing vessel conduit 32. When structured and positioned, effluent containing predominately the nanoparticles can be eluted from the flow filtration devices 35 and 36 and into a post-TFF conduit 37 that contains a post-TFF fluid stream.

As illustrated in FIG. 5, the apparatus also optionally includes a drug product formulation vessel 38. Said buffer exchange vessel captures the post-TFF fluid stream emanating from the post-TFF conduit and that comprises a fluid and nanoparticles. Said vessel can be open or closed. If closed, the vessel can comprise at least one input port in fluid connection with the post-TFF conduit 37 to permit entry of the post-TFF fluid stream. If open, the vessel can be structured and positioned to receive effluent from the post-TFF conduit 37.

As illustrated in FIG. 5, the drug product formulation vessel 38 permits buffer exchange of the nanoparticles so that the preferred concentration of excipients and/or other reagents and compositions can be added to the nanoparticles prior to finish and fill of pharmaceutical product/ingredients.

As illustrated in FIG. 5, the apparatus optionally further comprises a post-drug product formulation vessel conduit 41 that permits flow of the contents of the drug product formulation vessel 38 to a finish and fill apparatus 43. The apparatus may also optionally comprise yet another pump 42 structured and positioned to permit the pumping of a solution comprising nanoparticles 44 that has accumulated in said drug product formulation vessel.

Also, as mentioned in FIG. 5, it is also possible to retrieve some of the nanoparticles from the post-drug product formulation vessel conduit 41 for any of the quality control (QC) purposes one may wish to perform. Non-limiting examples of QC processes include sizing of the particles, confirming pH of the solution carrying the particles, the pH of the nanoparticles the zeta potential of the particles, the concentration of particles in the solution, the amount of API in the nanoparticles, etc.

In one aspect, the present disclosure also features a method comprising: a) treating a sample of nanoparticles comprising a synthetic polymer (e.g., PLGA) or mixture of polymers and an encapsulated and/or entrapped NPNAM, with a fluid (optionally comprising an acidic or basic solution (e.g., ammonia)) for a period of time suitable to dissolve or depolymerize the synthetic polymer (e.g., PLGA) and thereby release the encapsulated and/or entrapped NPNAM. In an embodiment, the method further comprises analyzing the sample for the presence, absence, and/or amount of the released NPNAM.

In an embodiment, the sample of nanoparticles to be treated further comprises a loading component. In some embodiments, the loading component is a nucleic acid. In one embodiment, the fluid comprising the acidic or basic solution (e.g., ammonia) is gaseous. In another embodiment, the fluid comprising the acidic or basic solution (e.g., ammonia) is an aqueous solution.

In another aspect, the present disclosure features a method of manufacturing, or evaluating, a nanoparticle or preparation of nanoparticles comprising providing a preparation of nanoparticles described herein, and acquiring, directly or indirectly, a value for a preparation parameter. In an embodiment, the method further comprises making the preparation of nanoparticles by a method described herein (e.g., the method illustrated by FIG. 4 or FIG. 5). In an embodiment, the method further comprises evaluating the value for the preparation parameter, by comparing it with a standard or reference value. In an embodiment, wherein responsive to the evaluation, the method further comprises selecting a course of action, and optionally, performing the action. For example, the method may comprise providing a preparation of nanoparticles comprising a NPNAM (e.g., a PNA oligomer) and a synthetic polymer (e.g., PLGA), acquiring a value for a preparation parameter (e.g., average nanoparticle size), evaluating the preparation the value of the preparation parameter by comparing it with a standard or reference value, selecting a course of action (e.g., selecting to administer the preparation of nanoparticles to a subject), and performing the action (e.g. administering the preparation of nanoparticles to a subject).

It is to be understood that not all (or even any) of the optional components of the apparatus must be present to operate. However, the apparatus is so configured to permit efficient preparation of nanoparticles formulated with encapsulated/entrapped NPNAM (and optionally one or more load components (e.g. nucleic acid(s)) and optionally provides for integrated removal of excess reagents as well as for post-production operations such as sterilization and finish and fill.

### e. General

In general, the process described herein entails a nanoprecipitation process (See for Example: WO2004/002453 for the formation of lipid nanoparticles), rather than a single or double emulsion process. One difference between the presently disclosed nanoprecipitation process and prior art processes is that in the presently disclosed process, the NPNAM is supplied in a water miscible organic solvent (or mixture of organic solvents) that may optionally comprise a relatively small amount of water. Thus, when making a nanoparticle that comprises both a nucleic acid and a NPNAM, the nucleic acid and the NPNAM are separated from each other and therefore unable to react to form aggregates or other secondary structures (immediately) prior to being available for encapsulation/entrapment into a nanoparticle, which is commonly an issue in other methods.

For clarity, the water miscible organic solvent may be suitable to dissolve the NPNAM at a concentration sufficient for nanoparticle formation. The water miscible organic solvent may also dissolve the synthetic polymer. In some embodiments, a water miscible solvent is chosen so that the second solvent supply (i.e. the solution comprising the NPNAM in organic solvent) can immediately and completely mix with the first solvent supply (an aqueous solution) at the junction (i.e. when the two solutions are put in contact). In this way, the presence of a first solvent supply and second solvent supply in the presently disclosed process differs from other emulsion processes, as those processes require use of an organic solvent that is not miscible with water, or in other words, a choice of solvents that do not mix. Because the products of the process will, in some cases, be pharmaceutical products, preferences for selection of solvents may also include review of Food and Drug Administration (FDA) data and recommendations or guidelines for organic solvents used in the formulation of pharmaceutical products/ingredients, with a view towards avoiding those solvents that are less preferred by the FDA. Non-limiting examples of suitable water miscible organic solvents include (but are not limited to): acetone, acetonitrile, methanol, ethanol, isopropanol, t-butyl alcohol, dimethyl sulfoxide, diethylene glycol dimethyl ether, 1,4-dioxane and ethylene glycol.

In some embodiments, the first solvent supply (or first solvent) is an aqueous solution. It can be plain water. It can be a buffer. It can be an unbuffered salt solution. It can comprise one or more load components (e.g. nucleic acid(s)) that may be incorporated with a NPNAM in the same nanoparticle. It can optionally contain another water-soluble compound (e.g. a small molecule) that one may wish to encapsulate/entrap in a nanoparticle.

In some embodiments, the second solvent supply (or second solvent) is a made from a water-miscible organic solvent. A polymer (e.g., a synthetic polymer) and at least one NPNAM may be dissolved in the water miscible organic solvent. The polymer (e.g., synthetic polymer) can be selected as the material to form the nanoparticle structure and encapsulate/entrap the active ingredients, such as the NPNAM. The second solvent supply can optionally contain additional NPNAM(s) and one or more other organic solvent-soluble compounds (e.g. a small molecule) that one may wish to encapsulate/entrap in a nanoparticle.

A non-limiting list of suitable polymers (e.g., synthetic polymers) that can be used to produce the second solvent supply include polylactic acid (PLA), polyglycolic acid, (PGA), poly(lactic-co-glycolic acid) (PLGA), poly(4-hydroxy-L-proline ester), other degradable polyesters, polyanhydrides, poly(ortho)esters, polyesters, polyurethanes, poly(butic acid), poly(valeric acid), poly(caprolactone), poly(hydroxyalkanoates), poly(lactide-co-caprolactone), poly(amine-co-ester) polymers, PEGylated polymers (e.g., a PEGylated PLGA), or a combination of any two or more of the foregoing.

A preferred NPNAM used in the practice of this invention is the peptide nucleic acid. In some embodiments, the NPNAM is a tail-clamp PNA (tcPNA—See FIG. 2 for a discussion of tail clamp molecules). Tail-clamp PNAs have been shown to be useful in gene editing applications (See: Bahal et al, infra). In an embodiment, the NPNAM used in a nanoparticle comprises a structure of Formula (I), as described herein.

After the initial mixing of the first solvent supply and second solvent supply, the solution so formed may be further diluted. Further dilution is thought to increase the stability of the initially formed nanoparticles. It also reduces the concentration of salt/buffer and free floating (i.e. not encapsulated/entrapped) biomolecules. Thus, practice of the methods disclosed herein generally will involve adding a dilute to the mixture resulting from mixing the first solvent supply and the second solvent supply.

Applicant has observed that processing nanoparticles through a diafiltration process, such as tangential flow filtration (TFF), can result in significant losses of nanoparticles, presumably through shearing or through adsorption cause by interactions of the nanoparticles with the surfaces of the TFF devices. To abate this effect, Applicant has found that addition of a surface stabilizer, such as a sugar (e.g. trehalose), to the nanoparticles prior to their processing through a TFF can stabilize the nanoparticles and thereby provide for a significantly increased yield of nanoparticle product out of the TFF device. The surface stabilizer can be any compound that improves the yield of the nanoparticles getting through the TFF device or devices. In some embodiments, the surface stabilizer can be trehalose, sucrose, cyclodextrin or a combination of two or more of the foregoing.

Tangential Flow Filtration (TFF) is one form of diafiltration. Applicant has found that flow through one or more TFF devices can be used to remove the buffer, organic solvent as well as residual NPNAM and nucleic acid (NA) (if present) or at least lower the concentration of these components to acceptable levels, to thereby provide a solution containing primarily only the so formed nanoparticles. Consequently, this permits in-line processing of the nanoparticles to a high level of purity and is amenable to scaling.

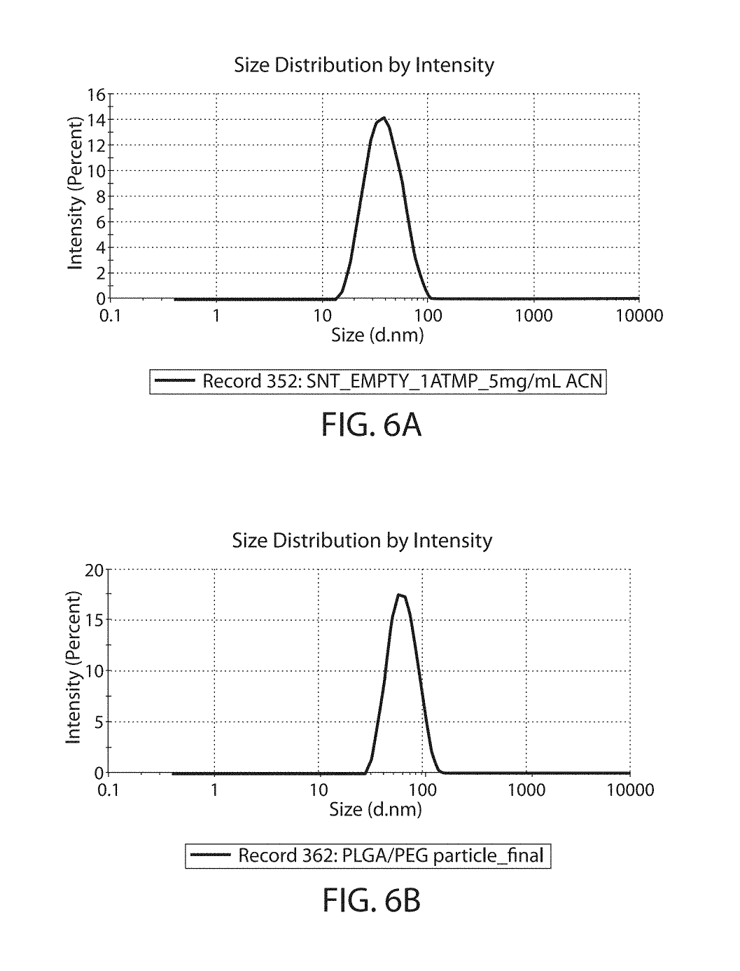

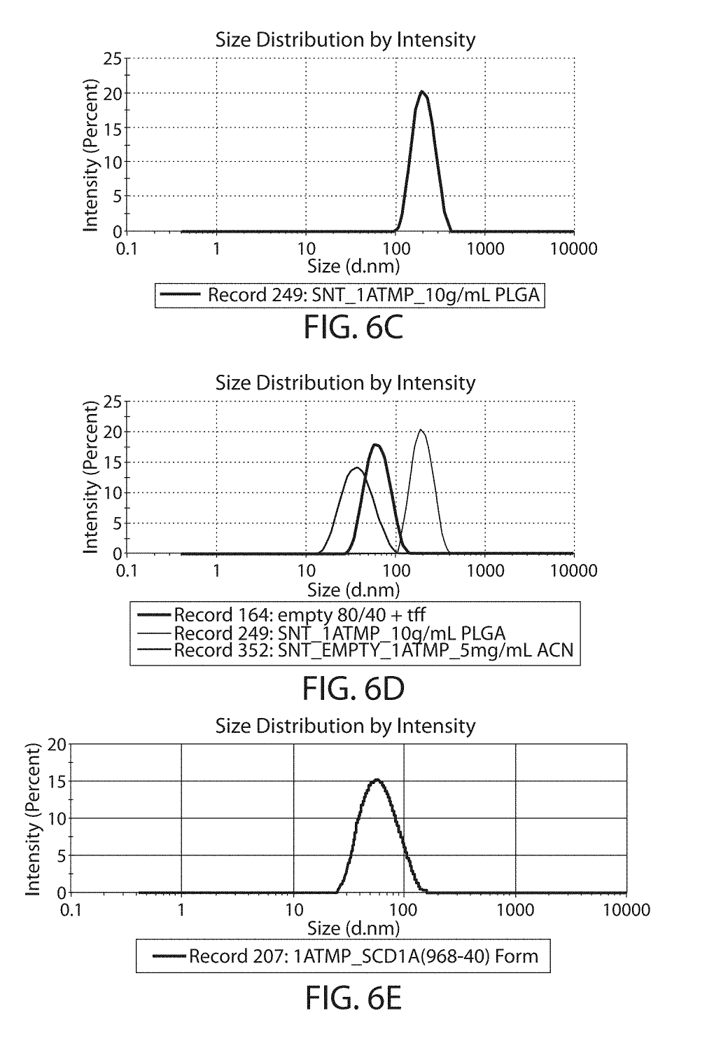

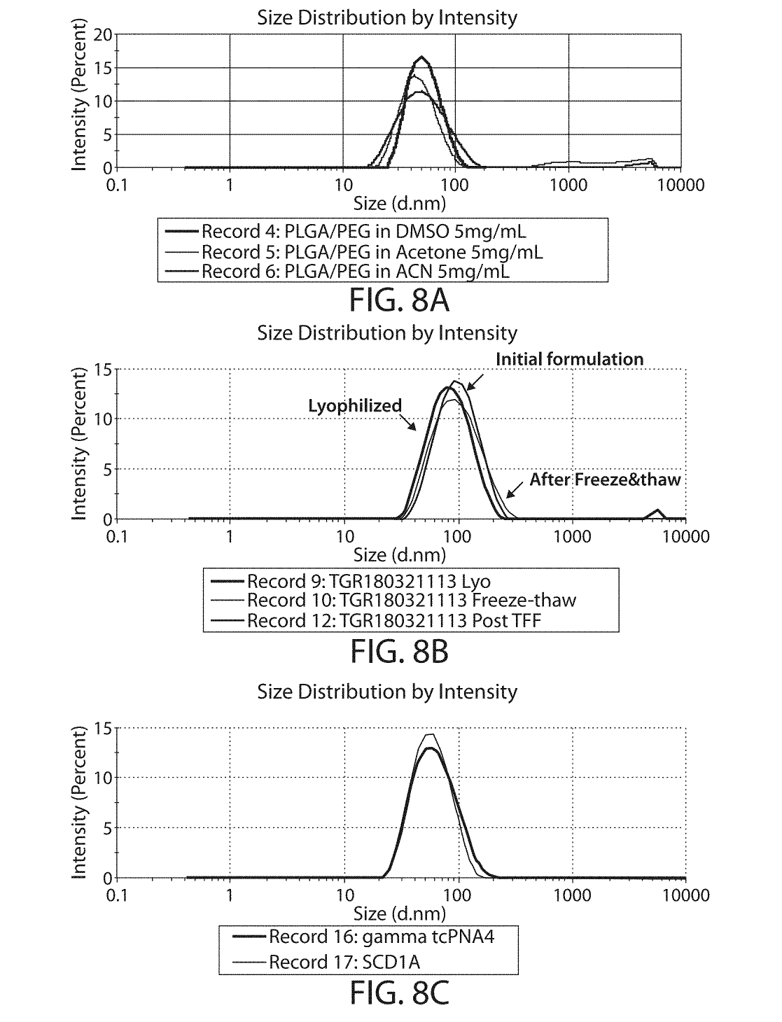

With the nanoparticles formed and relatively pure, the nanoparticles may then be used to formulate the drug product. Applicant has found that though adjustment of concentrations of input materials as well as choice of solvents and input APIs, the nanoparticle sizes can be tuned (See: Example 1 and FIGS. 6A to 6E, 7A, 7B, 9A-9E, 8A, 8B and 8C) to produce nanoparticle of desired size, polymer content and API content. Nanoparticles can be formulated to be remarkably stable to processes such as lyophilization as well as freezing and thawing (See: FIG. 8B). Further, the particles can be made to be suitable for sterilization by filtration, e.g., through a 0.22 μm filtration device, which is an industry standard for sterilization. Again, sterilization by filtration permits in-line processing of the nanoparticles as they are prepared for final formulation and packaging.

Before or after sterilization, any excipients, cyropreservative or other additives can be added to the nanoparticles before they are packaged for use. These additives can be added to a vat of the nanoparticles or they can be added in-line. Regardless of how they are added, this can be done in a quick and efficient manner to put the nanoparticles in suitable condition for packaging for use.

When finally formulated for packaging (and sterile, if applicable), the nanoparticles can be transferred to a finish and fill machine wherein the nanoparticles are packaged for dosing of subjects/patients. Again, all this can be performed in-line. If necessary, the packaged products can be stored under suitable conditions prior to use. In some cases, storage may require freezing of the sample and storage at 0° C., at −20° C., at −80° C. or even lower.

It should not go unsaid that nanoparticles can be diverted from any or all points in the process for analysis and quality control.

With specific reference to FIGS. 6A to 6E, FIG. 6A illustrates the size distribution by intensity of blank (empty—i.e. no API) PLGA nanoparticles formed using 5 mg/mL PLGA/PEG in acetonitrile as the water miscible organic solvent and PLGA/PEG as the polymer. These particles are roughly 45 nm on average in size with a distribution running from approximately 20 nm to 100 nm in diameter in size.

With reference to FIG. 6B, the size distribution by intensity analysis reveals that for this formulation, the PLGA/PEG nanoparticles are roughly 60 nm on average in size with a distribution running from approximately 30 nm to 110 nm in diameter in size.

With reference to FIG. 6C, the size distribution by intensity for yet another formulation prepared using 10 mg/mL PLGA as the polymer produced nanoparticles that are roughly 110 nm on average in size with a distribution running from approximately 100 nm to 130 nm in diameter in size.

With reference to FIG. 6D, it is clear from the overlay of several different formulations, it is possible to produce nanoparticles of significantly different size characteristics depending on choice of water miscible organic solvent and starting concentration of polymer present in said water miscible organic solvent.

With reference to FIG. 6E, the size distribution by intensity analysis is presented for PLGA nanoparticles loaded with PNA (PNA-1) and donor DNA (SEQ ID No: 2) and used in Example 3, below, to elicit gene editing/correction of cells bearing the SCD mutation. For this formulation, PLGA nanoparticles are roughly 60 nm on average in size with a distribution running from approximately 35 nm to 110 nm in diameter in size.

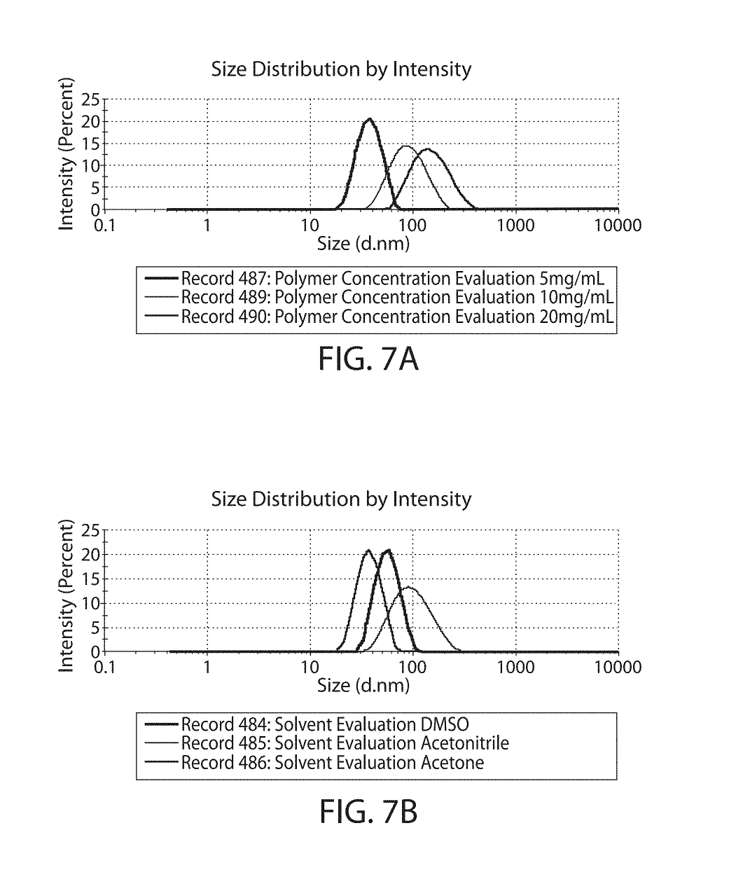

In order to examine how variation in formulation conditions would impact size distribution by intensity results, various parameters were systematically examined. For example, FIG. 7A illustrates how the size distribution by intensity of a formulation can be changed depending of the concentration of a polymer (in this case PLGA/PEG present in the water miscible organic solvent when all other possible variables are held constant). As the graph illustrates, as the polymer concentration increases, the average nanoparticle size increases.

Similarly, FIG. 7B illustrates that different solvents can be used to more precisely tune particle size as the data demonstrates that, with other possible variables held constant, acetone provides smaller nanoparticles, as compared with DMSO (second smallest nanoparticles) and acetonitrile (largest nanoparticles).

With reference to FIG. 8A, three traces are seen, one for each of three different batches of PLGA/PEG nanoparticles loaded with a PNA and donor DNA formed using 5 mg/mL polymer in the water miscible organic solvent in each of DMSO, acetone or acetonitrile (ACN) as the water miscible organic solvent. As can be seen from the traces, the size distribution of the nanoparticles so formed does not seem to be all that different regardless of the water miscible solvent chosen.

With reference to FIG. 8B, three traces are seen, all for the same batch of particles, but each after: (i) initial formation; (ii) after lyophilization and finally, (iii) after a freeze/thaw cycle. As can be seen from the data, the steps of lyophilization and freeze/thaw does not significantly affect the nanoparticle size distribution by intensity for these particles.

With reference to FIG. 8C, two traces are shown for PLGA nanoparticles formed using the same procedure except that in each different formulation, a different combination of PNA and load component (e.g., donor DNA) was used. More specifically, in one formulation, the PNA was PNA-1 and the donor DNA was SEQ ID NO: 2. This formulation is prepared for correction of the sickle cell disease mutation. In the second formulation, the PNA was PNA-2 and the donor DNA was SEQ ID NO: 4. This formulation is prepared for correction of a mutation that causes beta thalasemia. As can be seen from the data, the size distributions by intensity are similar regardless of the nature of the active pharmaceutical ingredients that are loaded.

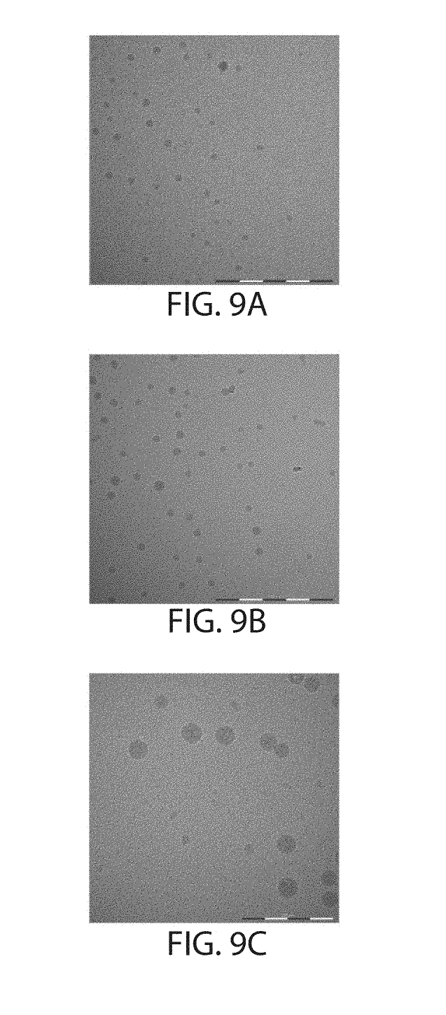

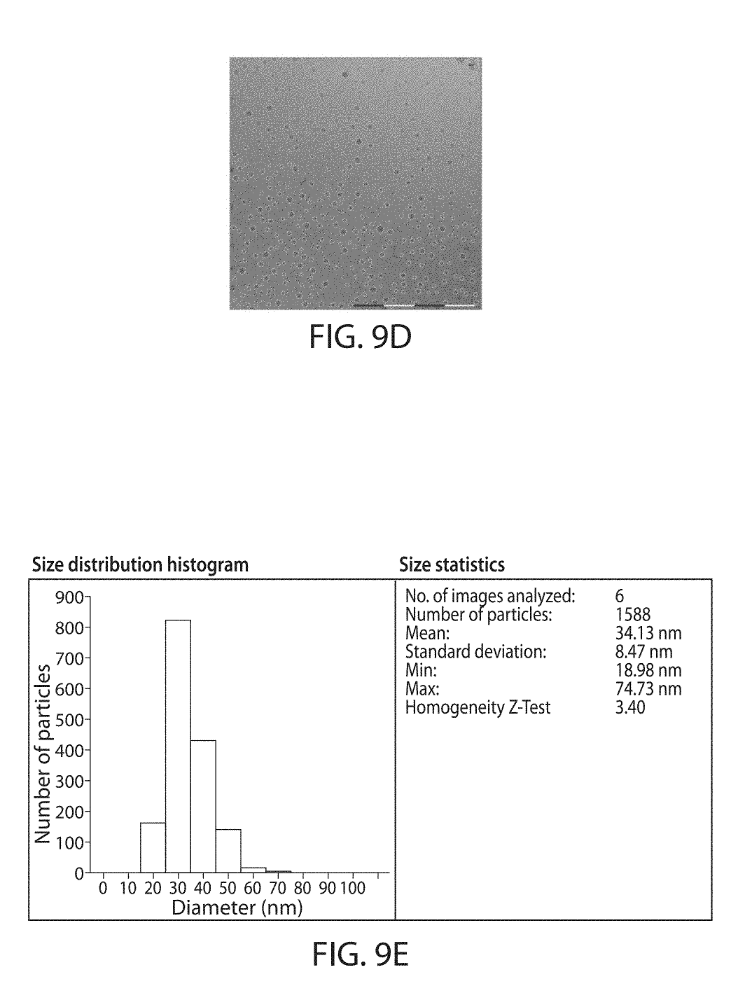

With reference to FIGS. 9A-9D, CyroTEM images of nanoparticles formed using the novel methodology described herein are shown. These images show that the nanoparticles formed are generally spherical and for this batch, the size distribution is between 20 nm and 60 nm in diameter, with most of the nanoparticles being about 30 nm in diameter (FIG. 9E).

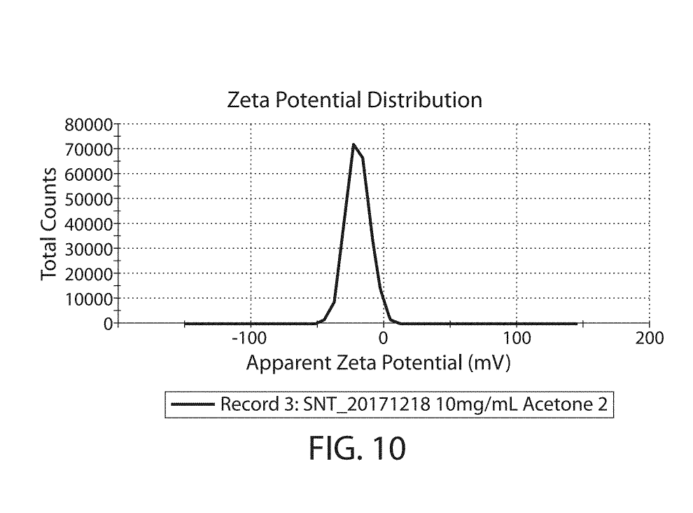

As is common for nanoparticles used in biological applications, it is typical to analyze the particles for zeta potential. FIG. 10 is illustrative of a formulation prepared using 10 mg/mL PLGA and acetone as the water miscible organic solvent and has a zeta potential of approximately −20 on average.

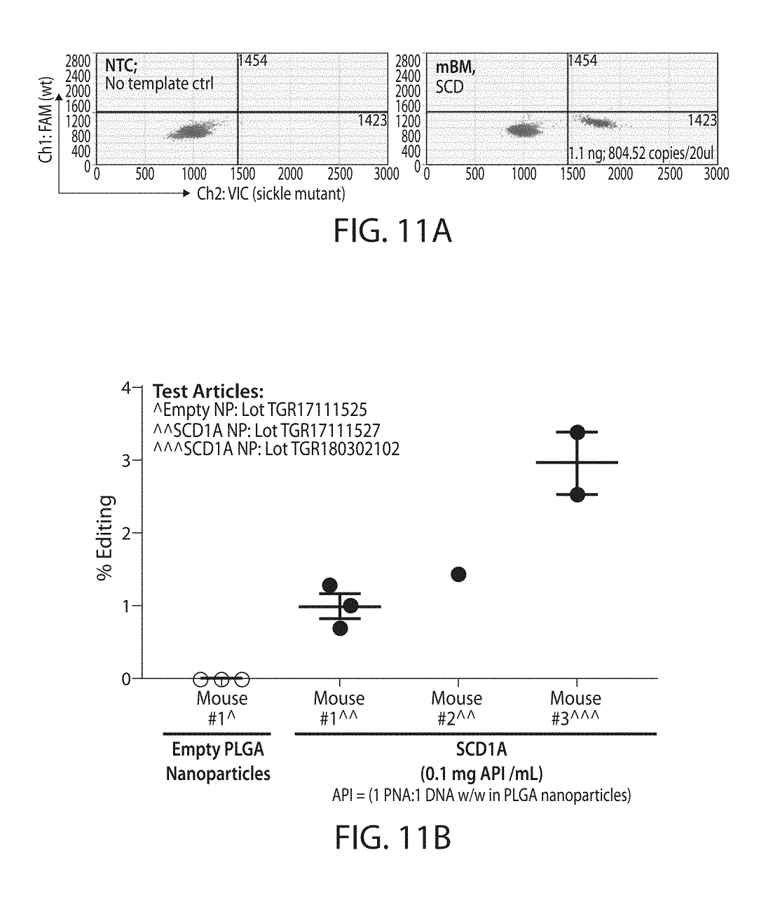

From the foregoing, it should be apparent that Applicant has discovered a robust process that is scalable and very adaptable. It can be used to produce nanoparticles comprising at least one or more NPNAM(s), but optionally one or more other active ingredients such as one or more load component (e.g. nucleic acid(s)). The process can be used to produce nanoparticles suitable for gene editing as illustrated in Examples 3-5 and FIGS. 11A to 16. In some embodiments, the process is used to produce a gene targeting composition (e.g., a gene editing composition). The process if particularly efficient in that all steps can be performed sequentially in a flow through process using a properly designed apparatus from starting materials put into a first and second solution and running all the way to finish goods.

For example, FIG. 11A presents ddPCR data graphically for a gene editing sample, wherein a sample containing SCD mutant genes is run against a no template control. In this graph, a clear separation is observed for positive samples and blanks. FIG. 11B presents summarized data for ddPCR for various samples, all of which (expect for the empty particle control) exhibited about 1 to 3 percent gene editing.

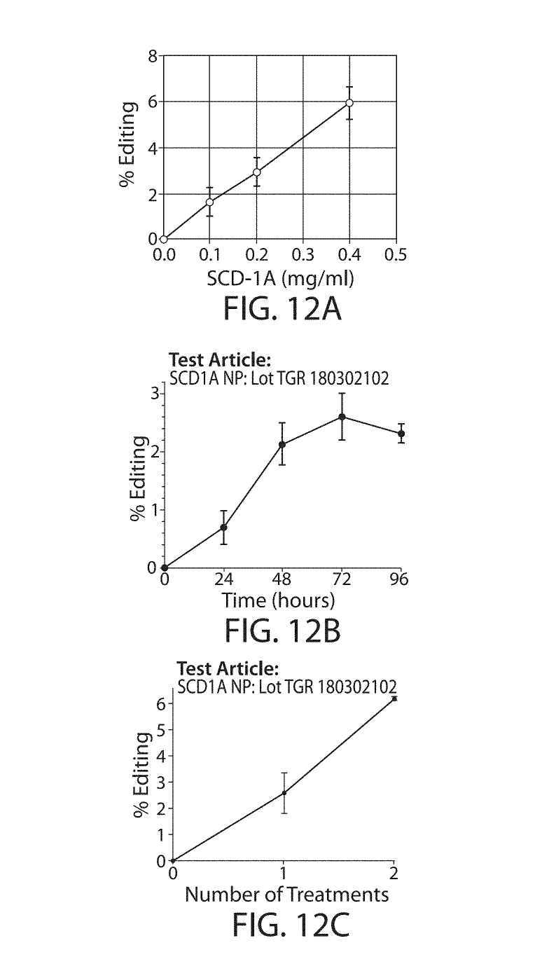

FIG. 12A illustrates a correlation between increasing dose and increasing gene editing. FIG. 12B illustrates that percent editing can be affected by time the nanoparticles are allowed to incubate with the target cells. FIG. 12C shows a correlation between an increase in the number of treatments with an increase in the percent gene editing.

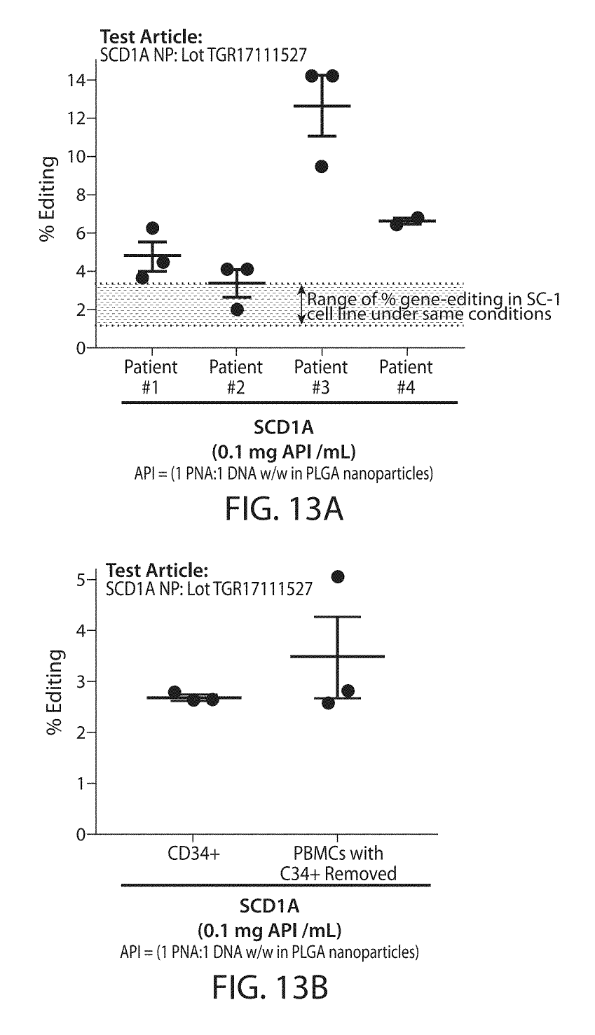

FIGS. 13A and 13B show gene editing data SCD in human cells, as compared with mouse cells. Generally, there was a larger observed percent gene editing in the human cells as compared with mouse cells.

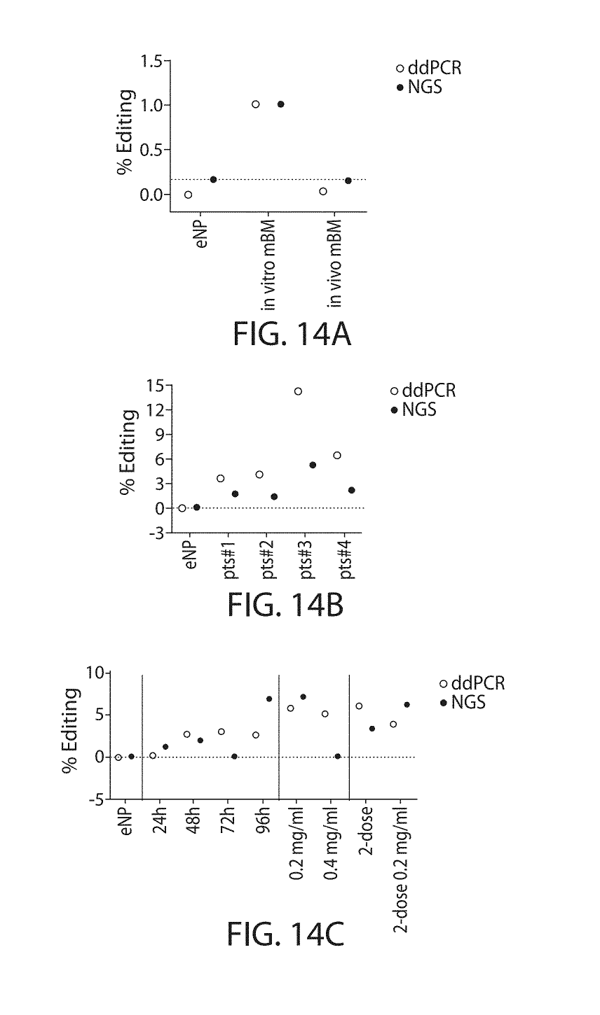

FIGS. 14A to 14C compare gene editing data for ddPCR vs NGS. The correction of mutation by gene-editing detected by ddPCR is further confirmed by NGS (FIGS. 14A to 14C). NGS is used to determine the gene sequences within the DNA samples, and the abundance of the corrected allele. Similar levels of gene correction are observed by both ddPCR and NGS.

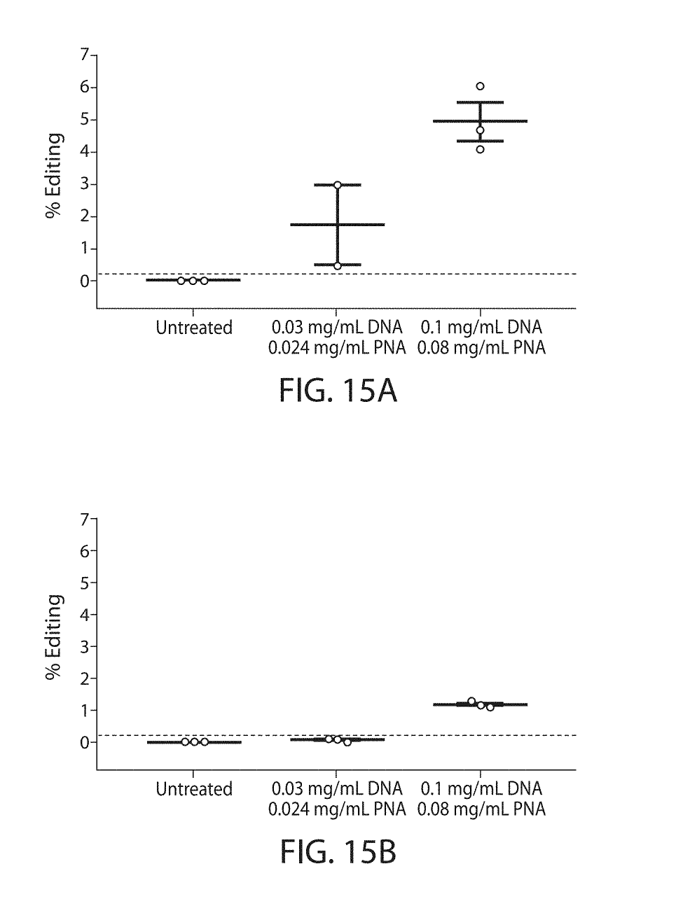

FIGS. 15A to 15B compare gene editing data (generated with ddPCR) for the same formulation, but where the cells were treated either with freshly made nanoparticles (FIG. 15A) or with the same formulation of nanoparticles that had been stored at about 4° C. for approximately 3 weeks before again being used to effect gene editing in vitro (FIG. 15B). The data indicates that while the efficacy of the formulation deteriorates over time under these storage conditions, the efficacy of the nanoparticles is not completely destroyed in that time frame.

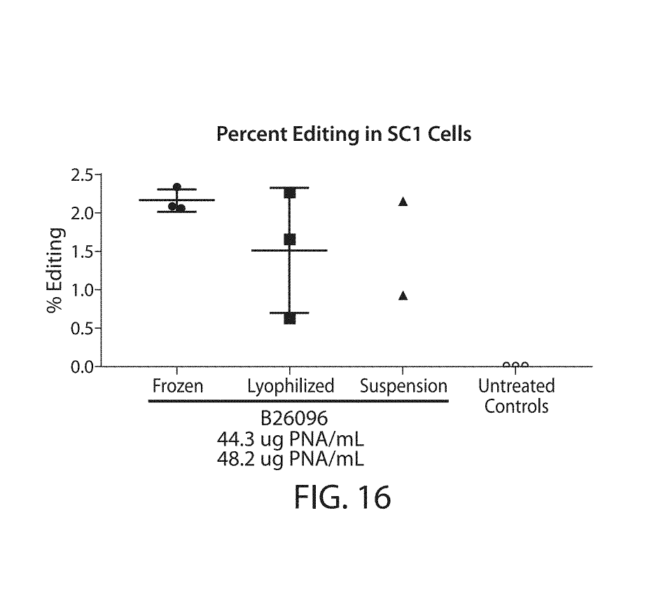

FIG. 16 compares gene editing data (generated with ddPCR) for the same formulation, wherein the formulation was split into three aliquots and each of the aliquots was subjected to a different storage condition (i.e. either Frozen, Lyophilized or Suspended) for about 24 hours prior to being used to generate the gene editing data. The data indicates that in all cases the sample exhibited gene editing after storage.

### f. Gene Targeting Compositions

The present disclosure further entails methods of altering a target nucleic acid using the nanoparticles and related preparations described herein. In one aspect, the present disclosure features a method of altering a target nucleic acid, comprising providing a preparation of nanoparticles described herein, e.g., comprising a NPNAM and/or a synthetic polymer described herein (e.g., a NPNAM of Formula (I) or a synthetic polymer of Formula (II)), or a preparation of nanoparticles made by a method described herein, e.g., as depicted in FIG. 4 or FIG. 5. The method of altering a target nucleic acid may further comprise contacting the NPNAM of a nanoparticle with a target nucleic acid under conditions sufficient to alter the target nucleic acid. In some embodiments, the method comprises administering a gene targeting composition (e.g., a gene editing composition).

The method of altering a target nucleic acid may be performed in an in vitro cell system, in a cell, or in vivo (e.g., in a subject, e.g., a human subject). In some embodiments, the method is performed in an in vitro cell free system. In some embodiments, the method is performed in a cell. The cell may be a cultured cell, e.g., a cell from a cell line, or may be a cell derived from a subject. In some embodiments, the method is performed in vivo, e.g., in a subject. The subject may be a mammal (e.g., a mouse, other non-human primate or a human).

In some embodiments, altering a nucleic acid comprises one or more of the following:
a) altering the state of association of the two strands of a target double stranded nucleic acid;
b) altering the helical structure of a target double stranded nucleic acid;
c) altering the topology in a strand of a target double stranded nucleic acid (e.g., introducing a kink or bend in a strand of the target double stranded nucleic acid);
d) recruiting a nucleic acid-modifying protein (e.g., enzyme), for example, a member of the nucleotide excision repair pathway, to a target double stranded nucleic acid. Exemplary members of the nucleotide excision repair pathway include XPA, RPA, XPF, and XPG, or a functional variant or fragment thereof;
e) cleaving a strand of a target double stranded nucleic acid; or
f) altering the sequence of a target double stranded nucleic acid. In some embodiments, the sequence of a target double stranded nucleic acid is altered to the sequence of a template nucleic acid. In some embodiments, the sequence of a target double stranded nucleic acid is altered from a mutant or disorder-associated sequence (e.g., allele) to a non-mutant or non-disease associated sequence (e.g., allele).

In some embodiments, altering a nucleic acid comprises two of (a)-(f). In some embodiments, altering a nucleic acid comprises three of (a)-(f). In some embodiments, altering a nucleic acid comprises four of (a)-(f). In some embodiments, altering a nucleic acid comprises five of (a)-(f). In some embodiments, altering a nucleic acid comprises each of (a)-(f). In some embodiments, altering a nucleic acid comprises (a). In some embodiments, altering a nucleic acid comprises (b). In some embodiments, altering a nucleic acid comprises (c). In some embodiments, altering a nucleic acid comprises (d). In some embodiments, altering a nucleic acid comprises (e). In some embodiments, altering a nucleic acid comprises (f).

A gene targeting composition (e.g., a gene editing composition) may also encompass multi-component systems where different components of the gene targeting composition are delivered individually (e.g. different APIs used in combination are delivered individually to the subject). In some embodiments, the NPNAM of the gene targeting composition is packaged into a single composition of matter for delivery to the subject (e.g. all APIs of the gene editing composition are loaded into a single nanoparticle).

The nanoparticle may promote a particular effect in a target nucleic acid sequence. For example, a nanoparticle when contacted with a target nucleic acid may allow binding of its component NPNAM to a target nucleic acid sequence. A nanoparticle when contacted with a target nucleic acid may provide a decrease in the melting point (Tm) of a target nucleic acid sequence (e.g., a decrease of about 0.5%, 1%, 5%, 10%, 20%, 30%, 40%, 50%, or more). A nanoparticle when contacted with a target nucleic acid may promote melting or dissociation of the strands of a target nucleic acid sequence (e.g., a melting or dissociation of about 0.5%, 1%, 5%, 10%, 20%, 30%, 40%, 50%, or more of the strands of the target sequence). A nanoparticle when contacted with a target nucleic acid, may allow its component NPNAM to cleave a target nucleic acid sequence (e.g., effect cleavage in about 0.5%, 1%, 5%, 10%, 20%, 30%, 40%, 50%, or more target nucleic acid sequences). A nanoparticle when contacted with a target nucleic acid may allow its component NPNAM and nucleic acid to edit a target nucleic acid sequence (e.g., edit about 0.5%, 1%, 5%, 10%, 20%, 30%, 40%, 50%, or more of the strands of the target sequence).

The extent of gene editing achieved by a gene editing composition (e.g., a preparation of a plurality of nanoparticles described herein) may be measured by any method known in the art. For example, the extent of gene editing may be achieved by polymerase chain reaction (PCR) or a particular sequencing method.

### 5. Various Embodiments of the Invention

With respect to this section 5 and the claims, it should be understood that the order of steps or order for performing certain actions is immaterial so long as the present teachings remain operable or unless otherwise specified. Moreover, in some embodiments, two or more steps or actions can be conducted simultaneously so long as the present teachings remain operable or unless otherwise specified.

In some embodiments, the present disclosure pertains to a method comprising: a) mixing at least a first solution and a second solution at a junction under conditions suitable to produce nanoprecipitation in a post-junction fluid stream comprising a post-junction fluid, wherein: (i) said first solution comprises a first solvent (e.g., water or an aqueous solution or buffer) optionally comprising up to 20% by volume of at least one water miscible organic solvent); and (ii) said second solution comprises a second solvent (e.g., water miscible organic solvent) comprising: (w) at least one neutral or positively charged nucleic acid mimic (NPNAM); (x) a polymer (e.g., a synthetic polymer) or mixture of polymers and (y) optionally up to 40% by volume of water. The method further comprises: (b) forming nanoparticles of the polymer (e.g., synthetic polymer) comprising the neutral or positively charged nucleic acid mimic(s) (NPNAM(s)) encapsulated and/or entrapped therein in the post-junction fluid stream. In some embodiments, the post-junction fluid stream is further diluted by addition of a diluent directly into said stream.

For clarity, it is to be understood that the polymer (e.g., synthetic polymer) used in performance of the method can comprise polymer mixtures, polymer blends and co-polymers. It is also to be understood that in some embodiments, a surfactant (e.g. polyvinyl alcohol) can be added to the first solution, the second solution or both the first solution and the second solution.

In some embodiments, the first solution can further comprise a nucleic acid and the nanoparticles so formed in the post-junction fluid stream comprise both nucleic acid(s) and the NPNAM(s) encapsulated and/or entrapped therein. The nucleic acid(s) can be a DNA oligomer, preferably of between 20 and 100 nucleotides in length. The nucleic acid can be an RNA oligomer. The nucleic acid can comprise one or more phosphorothioate linkages. In some embodiments, the nucleic acid can comprise at least two phosphorothioate linkages at each of its 3′ and 5′ termini. In some embodiments, the nucleic acid is an antisense agent.

In some embodiments, the neutral or positively charged nucleic acid mimic(s) (NPNAM(s)) is/are selected from the group consisting of: peptide nucleic acid, morpholino, pyrrolidine-amide oligonucleotide mimic, morpholinoglycine oligonucleotide and methyl phosphonate. It is to be understood that mixtures of two more different types of NPNAM can be used at the same time. For example, both a PNA and a morpholino can be mixed in the second solution. In some embodiments the NPNAM is a peptide nucleic acid. In some embodiments, the NPNAM is a PNA comprising a structure of Formula (I) (e.g., as described herein). In some embodiments, the peptide nucleic acid is a tail-clamp peptide nucleic acid (tcPNA).

According to the method the first solution can comprise from 0.05% to 20% organic solvent, wherein the organic solvent can be selected from the group consisting of: acetone, acetonitrile, methanol, ethanol, isopropanol, t-butyl alcohol, dimethyl sulfoxide, diethylene glycol dimethyl ether, 1,4-dioxane and ethylene glycol. Furthermore, in any of the various embodiments of the invention, the organic solvent can be selected from the group consisting of: acetone, acetonitrile, methanol, ethanol, isopropanol, t-butyl alcohol, dimethyl sulfoxide, diethylene glycol dimethyl ether, 1,4-dioxane and ethylene glycol. Some preferred but non-limiting examples of organic solvents include methanol, ethanol, acetone, acetonitrile, and dimethyl sulfoxide.

In some embodiments, the polymer (e.g., synthetic polymer) is selected from the group consisting of polymers, copolymers, mixtures of blends of: polylactic acid (PLA), polyglycolic acid, (PGA), poly(lactic-co-glycolic acid) (PLGA), poly(4-hydroxy-L-proline ester), other degradable polyesters, polyanhydrides, poly(ortho)esters, polyesters, polyurethanes, poly(butic acid), poly(valeric acid), poly(caprolactone), poly(hydroxyalkanoates), poly(lactide-co-caprolactone), poly(amine-co-ester) polymers, or a combination of any two or more of the foregoing. In some embodiments, the polymer (e.g., synthetic polymer) is PLGA or PLA or PLG, or mixtures, blends or co-polymers thereof. In some embodiments, the polymer (e.g., synthetic polymer) comprises a structure of Formula (II) (e.g., as described herein).

In some embodiments, the second solution can further comprise polyethylene glycol conjugated to a synthetic polymer, which can be incorporated into the nanoparticles to improve properties of the resulting nanoparticles, such as increased stability through the various steps of the process, including without limitation, cryostability. For example, the second solution can comprise from about 0.1% to about 5% PEG.

In some embodiments, the second solution can comprise up to about 20% water. The water content may assist in solubilizing one or more of the reagents added to said second solution, including the PLGA. For example, the second solution can comprise from 0.05% to 20% water.

According to the method, the nanoparticles are formed by nanoprecipitation in the post-junction fluid stream. It is known in the art that once formed, the nanoparticles can be further reduced in size and strengthen by dilution with water or aqueous buffer. Thus, in some embodiments, this method further comprises the step of: (c) diluting the post-junction fluid stream.

In some embodiments, the method contemplates use of a tangential flow filtration (TFF) step to remove certain impurities from the nanoparticles. It has been determined that introduction of a surface stabilizer to the solution comprising the nanoparticles prior to their introduction to the TFF permits improved recovery of nanoparticles. Without being bound to any theory, it is believed that the surface stabilizer binds to the outer surface of the nanoparticles and shields the surface of the nanoparticles from interactions with the surfaces of the TFF device, thereby avoiding shearing of the nanoparticles and/or adhesion of the nanoparticles upon interactions with said surfaces of the TFF device. Thus, in some embodiments, this invention pertains to both: (i) adding a surface stabilizer to a solution containing nanoparticles prior to performing TFF of said solution; and (ii) in some embodiments of the aforementioned method: (d) adding a surface stabilizer to stabilize the nanoparticles formed in the post-junction fluid stream. The surface stabilizer can be any composition that stabilized the nanoparticles to degradation and/or losses during performance of TFF. For example, the surface stabilizer can be a cryopreservative such as cyclodextrin, trehalose or sucrose.

In any case, to formulate a therapeutic nanoparticle, excess reagents may be removed from the nanoparticles (e.g., the preparation of nanoparticles), post formation. For example, an excess reagent may be removed by performing TFF. Thus, in some embodiments, the method further comprises the step of: (e) removing from the post-junction fluid stream the NPNAM(s) that is/are not encapsulated and/or entrapped in the nanoparticles. In some embodiments, the method further comprises the step of: (f) removing from the post-junction fluid stream the organic solvent.

Because it is anticipated that in some applications of the invention(s) disclosed herein, the nanoparticles so formulated can be used as gene editing compositions, in some embodiments, practice of the method can also comprise the step of: (g) sterilizing the post-junction fluid comprising the nanoparticles. Sterilization of the nanoparticles (and the solution in which they are suspended) would be a requirement before they could be introduced into a subject.

Again, because it is anticipated that in some applications of the invention(s) disclosed herein, the nanoparticles so formulated can be used as gene editing compositions, in some embodiments, practice of the method can also comprise the step of: (h) finish and filling a sterile container with the nanoparticles as this phrase is understood in the pharmaceutical industry for production of pharmaceuticals.

It is to be understood that each of steps (c) through (h) are optional steps such that one or more of them can be used in practice of the inventive method described herein. Thus, the inventive methods of this disclosure contemplate practice of steps (a) and (b) in combination with any one or more of steps (c) through (h).

It is also to be understood that in some embodiments, the first solution can comprise nucleic acid(s) and consequently the nanoparticles so formed by practice of steps (a) and (b), in combination with any one or more of steps (c) through (h) can comprise encapsulated and/or entrapped nucleic acid(s) and neutral or positively charged nucleic acid mimic(s).

In some embodiments, the nanoparticles so formed have average diameters ranging from 30 to 200 nm and neutral to negative −40 mv surface charge.

Applicant has determined (by deformulation (as described herein) of the nanoparticles comprising the NPNAM(s), and optionally nucleic acid(s)) that nanoparticles prepared according to practice of the aforementioned method produce nanoparticles having an extremely high loading of NPNAM as compared with nanoparticles formed using the prior art double emulsion process. Thus, in some embodiments, this invention pertains to a nanoparticle comprising: (a) a synthetic polymer or mixture of polymers; and (b) at least one neutral or positively charged nucleic acid mimic encapsulated and/or entrapped within said synthetic polymer, wherein, the amount of NPNAM(s) encapsulated and/or entrapped within the nanoparticle is greater than or equal to (on average of a sample of nanoparticles so produced) 2 percent (2%) by weight of NPNAM(s) to the total weight of the nanoparticle(s). In some embodiments, the amount of NPNAM(s) encapsulated and/or entrapped within the nanoparticle is greater than or equal to (on average of a sample of nanoparticles so produced) 2.5 percent (2.5%) by weight of NPNAM(s) to the total weight of the nanoparticle(s). In some embodiments, the amount of NPNAM(s) encapsulated and/or entrapped within the nanoparticle is greater than or equal to (on average of a sample of nanoparticles so produced) 3 percent (3%) by weight of NPNAM(s) to the total weight of the nanoparticle(s). In some embodiments, the amount of NPNAM(s) encapsulated and/or entrapped within the nanoparticle is greater than or equal to (on average of a sample of nanoparticles so produced) 5 percent (5%) by weight of NPNAM(s) to the total weight of the nanoparticle(s). In some embodiments, the amount of NPNAM(s) encapsulated and/or entrapped within the nanoparticle is greater than or equal to (on average of a sample of nanoparticles so produced) 7 percent (7%) by weight of NPNAM(s) to the total weight of the nanoparticle(s).

Thus, in some embodiments, the amount of neutral or positively charged nucleic acid mimic encapsulated and/or entrapped within the nanoparticle is between 2 to 5 percent (2-5%) by weight of NPNAM(s) to the total weight of the nanoparticle(s). In some embodiments, the amount of neutral or positively charged nucleic acid mimic encapsulated and/or entrapped within the nanoparticle is between 2.5 to 5 percent (2.5-5%) by weight of NPNAM(s) to the total weight of the nanoparticle(s). In some embodiments, the amount of neutral or positively charged nucleic acid mimic encapsulated and/or entrapped within the nanoparticle is between 3 to 5 percent (3-5%) by weight of NPNAM(s) to the total weight of the nanoparticle(s). In some embodiments, the amount of neutral or positively charged nucleic acid mimic encapsulated and/or entrapped within the nanoparticle is between 2 to 7 percent (2-7%) by weight of NPNAM(s) to the total weight of the nanoparticle(s). In some embodiments, the amount of neutral or positively charged nucleic acid mimic encapsulated and/or entrapped within the nanoparticle is between 2.5 to 7 percent (2.5-7%) by weight of NPNAM(s) to the total weight of the nanoparticle(s). In some embodiments, the amount of neutral or positively charged nucleic acid mimic encapsulated and/or entrapped within the nanoparticle is between 3 to 7 percent (3-7%) by weight of NPNAM(s) to the total weight of the nanoparticle(s). When reference is made to a composition being a certain % by weight of a nanoparticle herein, it is to be understood that such calculation of % by weight is to be made considering only the API (e.g. NPNAM(s) and load component(s)) and the synthetic polymer(s) such that other compounds and reagents that might be incorporated into the nanoparticle (such as water, salt and sugars) are not considered in such calculation of % by weight.

As previously described, practice of the disclosed novel method can comprise nanoparticles comprising both NPNAM(s) and nucleic acid(s). Thus, in some embodiment, this invention contemplates and all forms of said nanoparticle can further comprise at least one nucleic acid encapsulated and/or entrapped therein. In some embodiments, the nucleic acid is a DNA oligomer of between 20 and 100 nucleotides in length. In some embodiments, the nucleic acid comprises at least two phosphorothioate linkages at each of its 3′ and 5′ termini. In some embodiments, the nucleic acid is an antisense agent. In some embodiments, the nucleic acid is an RNA oligomer.

According to the invention, the synthetic polymer of the nanoparticle can be selected from the group consisting of polymers or copolymers of: polylactic acid (PLA), polyglycolic acid, (PGA), poly(lactic-co-glycolic acid) (PLGA), poly(4-hydroxy-L-proline ester), other degradable polyesters, polyanhydrides, poly(ortho)esters, polyesters, polyurethanes, poly(butic acid), poly(valeric acid), poly(caprolactone), poly(hydroxyalkanoates), poly(lactide-co-caprolactone), poly(amine-co-ester) polymers, or a PEGylated polymer (e.g., PEGylated PLGA), or a combination of any two or more of the foregoing. In some embodiments, the synthetic polymer comprises a structure of Formula (II), e.g., as described herein.

According to the invention, the neutral or positively charged nucleic acid mimic(s) can be selected from the group consisting of: peptide nucleic acid, morpholino, pyrrolidine-amide oligonucleotide mimic, morpholinoglycine oligonucleotide and methyl phosphonate. In some embodiments the neutral or positively charged nucleic acid mimic is a peptide nucleic acid (PNA). In some embodiments, the NPNAM is a PNA comprising a structure of Formula (I), e.g., as described herein. In some embodiments, the PNA is a tail-clamp peptide nucleic acid (tcPNA).

In some embodiments, the nanoparticles further comprise polyethylene glycol (PEG) conjugated to a polymer. Inclusion of PEG in the nanoparticles can improve the long-term stability of cryopreserved nanoparticles.

In some embodiments, the diameter of the nanoparticles can range from 30 to 200 nanometers.

In some embodiments, this invention is also directed to an apparatus suitable for practice of the novel methodology disclosed herein, to thereby produce the novel nanoparticles as disclosed herein. Thus, in some embodiments, this invention pertains to an apparatus comprising: (a) a first solvent supply comprising at least water or an aqueous buffer; (b) a second solvent supply comprising at least one water miscible organic solvent and at least one neutral or positively charged nucleic acid mimic; (c) a junction to which the first solvent supply and second solvent supply are in fluid connection; and (d) a post-junction conduit suitable to contain a post-junction fluid stream; wherein said junction permits mixing of solvent forced through said junction simultaneously from the first and second solvent supplies and into the post-junction conduit.

As previously discussed, in some embodiments, practice of the novel methods disclosed herein produce nanoparticles comprising both NPNAM(s) and nucleic acid(s). Thus, in some embodiments, the first solvent supply of the novel apparatus further comprises at least one nucleic acid.

As previously discussed, in some embodiments, the fluids comprising freshly produced nanoparticles formed by nanoprecipitation are further diluted. Thus, in some embodiments, the apparatus of this invention can further comprise a second junction that fluidly connects a diluent supply to the post-junction conduit to thereby permit dilution of the contents of the post-junction fluid stream.

In some embodiments, the apparatus of this invention can further comprise a mixing vessel positioned to capture the post-junction fluid stream that exits the post-junction conduit. In some embodiments, the apparatus of this invention can still further comprise an input port fluidly connected to said mixing vessel and positioned and suitable to introduce a surface stabilizer to the contents of said mixing vessel. In some embodiments, said mixing vessel can further comprise an exit port, structured and positioned to permit fluid contents of the mixing vessel to exit the mixing vessel and flow into a post-mixing vessel conduit.

In some embodiments, the apparatus of this invention can further comprise a tangential flow filtration (TFF) device in fluid communication with the post-mixing vessel conduit. In some embodiments, the apparatus of this invention can further comprise a buffer exchange vessel structured and positioned to receive fluid exiting the post-mixing vessel conduit, and if so fitted, the fluid so filtered by the TFF.

In some embodiments, the apparatus of this invention can further comprise a sterilization device structured and positioned to sterilize fluid within or that has exited the post-mixing vessel conduit. For clarity, this includes fluid exiting the post-mixing vessel conduit filtered as by the TFF, if the apparatus is so fitted.

Because it is contemplated that in some cases, the nanoparticles so produced using this apparatus might be used as a therapeutic agent, in some embodiments, the apparatus of this invention can further comprise a finish and fill apparatus in fluid connection with the buffer exchange vessel.

As discussed above, Applicant has been able to determine the amount of a specific NPNAM within a formed nanoparticle (e.g. composed primarily of PLGA) by deformulation of said nanoparticles and analysis of the NPNAM recovered therefrom (See: Example 2). From this analysis, Applicant has been able to determine that practice of the disclosed methods using the disclosed apparatus can produce nanoparticles that comprise much higher loadings of a NPNAM than can be achieved with the double emulsion process.

Accordingly, this invention further relates to a method comprising: (a) treating a sample of nanoparticles composed primarily of PLGA and comprising encapsulated and/or entrapped neutral or positively charged nucleic acid mimic, and optionally a nucleic acid, with a fluid comprising ammonia for a period of time suitable to depolymerize the PLGA and thereby release the encapsulated and/or entrapped biopolymers; and (b) analyzing the sample for the presence, absence and/or amount of the released neutral or positively charged nucleic acid mimic(s), and optionally nucleic acid(s). According to the invention, the fluid comprising ammonia can be gaseous or an aqueous solution.

In still another embodiment, this invention pertains to method for gene editing. Said method comprises: (a) contacting cells in culture or an organism with a nanoparticle or nanoparticles described herein; and (b) analyzing a sample of cells or tissue from the organism to determine if gene editing occurred in said cells or tissue. In some embodiments, the contacting is performed by injection or infusion of the nanoparticle(s) into the bloodstream of the organism. In some embodiments, the contacting is performed by injection or infusion of the nanoparticle(s) directly into tissue of the organism. In some embodiments, the contacting is performed by adding the nanoparticles to a cell culture. In some embodiments, the analyzing of the sample of cells or tissue is performed using digital drop polymerase chain reaction (ddPCR) or by next generation sequencing (NGS) to thereby determine the percent editing of the cells or tissue.

### 6. Advantages of the Invention

Very high loading of NPNAM (e.g. PNA) within the nanoparticle can be achieved as shown by deformulation of the nanoparticles. Loading can be as high as 25 times (often 10-25 times) that which can be achieved with the double emulsion process.
Very good control of particle size and consistent loading of nanoparticles.
Very amenable to scaling to produce amounts of nanoparticles needed for therapeutic applications.
Smaller nanoparticles with tighter size distribution as compared with what can be achieved with the double emulsion process

### 7. Examples

Aspects of the present teachings can be further understood in light of the following examples, which should not be construed as limiting the scope of the present teachings in any way. Furthermore, it should be readily apparent to those of skill in the art that the following general procedures can be altered by variations on solvent, volumes and amounts of reagents in various steps to achieve optimal results for a particular compound without deviating from the scope and intent of the following guidance.

### Abbreviations

WFI: Water for injection

PLGA: Poly(DL-lactide-co-glycolide)

mPEG-PLA: Methoxy Poly(ethylene glycol)-b-Poly(D,L-lactide-co-glycolide)

MWCO: Molecular weight cut-off

TFF: Tangential flow filtration

### PNA and DNA Oligomers Used in the Examples:

The sequences for PNA and DNA oligomers used in these experiments were:

SCD1A PNA:
H-Lys Lys Lys j j t j t t j PEG3 C t T c T c C a
C a G g A g T c A g Lys Lys Lys-NH₂(PNA-1)-
Molecular Weight is approximately: 8681 g/mole
SCD1A DNA:
(SEQ ID NO: 2)
T*T*G* CCC CAC AGG GCA GTA ACG GCA GAC TTC TCC TCA
GGA GTC AGG TGC ACC ATG GTG TCT GT*T* T*G-Molecular
Weight is approximately 9984 g/mole
tcPNA4 PNA:
H-Lys Lys Lys j t t t j t t t j t j t PEG3 t C t C
t T t C t T t C a G g G c A Lys Lys Lys NH₂(PNA-2)
tcPNA4 DNA:
(SEQ ID NO. 4)
A*A*A* GAA TAA CAG TGA TAA TTT CTG GGT TAA GGC AAT
AGC AAT ATC TCT GCA TAT AAA *T*A*T

For the PNA oligomer, Lys refers to the amino acid, L-lysine (but D-lysine or a racemic mixture could be used as a substitute for one or more lysine moieties), j refers to a classic PNA monomer comprising the nucleobase pseudoisocytosine, PEG3 is a long chain linker construct of formula: —NH—(CH₂CH₂O)₃CH₂CO—, and the PNA subunits using an upper-case letter contain a gamma miniPEG substituent of formula (—CH₂—(OCH₂CH₂)—OH) as compared with those having a lower-case letter which are based on the classic PNA backbone (i.e. aminoethyl glycine backbone; See: Sahu et al., infra) for a discussion of the preparation and properties of gamma miniPEG PNAs. The asterisk shown in the DNA oligomers indicate internucleoside linkages that are phosphorothioates instead of phosphates. Polymers were obtained from commercial sources such as PolySci Tech and Durect Corp.

Droplet digital PCR (ddPCR) was performed with Bio-Rad QX200 using primers and probes as described below. ddPCR is a quantitative PCR method useful for the detection and measuring the amount of rare genetic variant in a DNA sample. This was achieved by partitioning DNA molecules in a sample, mixing with PCR reagents, into nanoliter-sized droplets formed in a water-oil emulsion. These individual droplets functioned as an individual PCR sample reaction.

For the quantification of the amount of rare genetic variant in a DNA sample, the number of droplets without DNA, droplets positive for rare variant allele, and droplets positive for WT allele were measured fluorescently by the ddPCR reader, and the amount of rare variant allele was measured based on the Poisson distribution and the number of these droplets. More information on droplet digital PCR can be found at the BioRad website (www.biorad.com) as they manufacture instruments and reagents for performing this technique.

Amplicons were prepared using PCR and primers designed around SCD mutation in human hemoglobin gene (Forward: 5′-TTGTAACCTTGATACCAACC-3′ (SEQ ID NO: 8) and Reverse: 5′-CTTACATTTGCTTCTGACAC-3′ (SEQ ID NO: 9), PCR conditions: 95° C., 3 min; ×35[95° C., 30 sec; 49.6° C., 30 sec; 72° C., 1 min]; 72° C., 10 min; 4° C. forever). PCR products were subjected to column clean-up (QIAquick Qiagen) and amplicons were evaluated on Qubit and later on 2% gel for size and purity. IIIumina TruSeq Paired-End Sequencing workflow was used to prepare libraries using purified amplicons as starting material. Samples were then sequenced on IIIumina MiSeq (2×150 bp) platform (merged paired reads). Unique nucleotide sequences in the region of interest were identified and a relative abundance was calculated for each unique sequence. In edited samples, variant abundance of unique sequences with correction of mutant A to wild-type T can be calculated. and shown in FIG. 15A (next to percent editing measured using ddPCR on genomic DNA of these samples).

### Example 1: General Process for Preparation of Nanoparticles

PNA/DNA/Polymer nanoparticles were prepared by combining: (a) 0.125 mg/mL of DNA dissolved in 100 mL of water (Solution A); with (b) 50 mL of solution comprising the components of Table 1 dissolved in 9/1 water miscible organic solvent/water (Solution B; for example: 9/1 acetone/water or 9/1 acetonitrile/water or 9/1 DMSO/water, as the case may be.

TABLE 1
Composition of 9/1 acetone/water solution used to
form Polymer/PNA/DNA nanoparticles

| | PLGA* | PLGA-PEG2000* | PNA |
|---|---|---|---|
| MW (Da) | 41000 | 22000 | ≈10,000** |
| Mol (μmol) | 6.03 | 0.12 | 1.25 |
| wt (mg) | 247.29 | 2.71 | 12.5 |

*These calculations were used to formulate nanoparticles at 5 mg/mL. Suitable adjustments were made to formulate nanoparticles with 10 mg/mL and 20 mg/mL (See: FIG. 7A)
**Actual molecular weights for each PNA oligomer are provided above.

Solution A and Solution B were mixed by flowing them simultaneously through a T-junction using syringe pump at a combined flow rate of 120 mL/min to form an initial polymer suspension comprising encapsulated DNA/PNA. This suspension was further mixed with 2× volume of water through another T-junction using a peristaltic pump, which further reduced acetone concentration to about 10% and resulted in a final volume of 450 mL of polymer suspension. Then, 22.5 mg of trehalose was added into the suspension to achieve a final trehalose concentration of about 5% (wt./vol). This mixed suspension was then subjected to tangential flow filtration (TFF) and diafiltrated against 900 mL of 5% trehalose, concentrated to a volume of about 50 mL, then diafiltrated against 400 mL of 5% trehalose and further concentrated to 10 mL of final volume. Particle size (approximately 60-90 nm), polydispersity index (0.083) and concentration was then measured. As illustrated in FIGS. 7A and 7B, the process described herein can be used with various concentrations of input synthetic polymer(s) (FIG. 7A) and with several different water miscible organic solvents (FIG. 7B and FIG. 8A).

Particle size and zeta potential were measured on a Malvern zetasizer according to manufacturer's instruction. Briefly, for particle size measurement, 10 μL particle suspension was added to 1 mL of WFI water in a transparent cuvette and the measurement was performed at 25° C. on a Malvern zetasizer. For zeta potential, a dip cell was used for the measurement.

Data provided in FIGS. 6A, 6B, 6C, 6D, 6E, 7A, 7B, 8A, 8B, 8C, 9A-9E and 10 are for nanoparticle batches performed using this process (with no API, just PNA, or PNA and DNA, as described) and analyzed on the Malvern zetasizer. It should also be understood that this process was successfully used with other organic miscible solvents, such as acetonitrile (ACN) and dimethylsulfoxide (DMSO). As can be seen in FIGS. 6D, 7A, 7B, 8A, 8B and 8C, variables such as starting concentrations of polymer, type of water miscible organic solvent, nature of API (e.g. DNA and/or PNA) and ratios of the input solvent supplies can be altered and adapted to achieve desired results (e.g. API concentrations and particle size). Performing the process by fine adjustment to these variables to achieve desired particle sizes, zeta potential and/or particle loading of API are certainly within the skill of the art when using the disclosure provided herein.

This nanoprecipitation process not only enables the scale-up production of PNA/DNA loaded PLGA particles, but also improved the loading of PNA/DNA inside PLGA particles. The smaller particle size due to the fine control of the mixing step has also made it feasible to achieve sterile product by 0.22 um filtration.

Finally, the robustness of nanoparticles that can be formed using this novel process is illustrated in FIG. 8B, wherein it is shown that particles can be lyopyhilozed and subjected to at least one freeze/thaw cycle without significant change in particle size.

### Example 2: Protocol for Deformulation of PLGA Particles

### Principle of the Procedure

A known amount of PLGA particles are reacted with concentrated aqueous ammonia to convert the PLGA into water soluble glycoamide and lactamide (possibly other compounds as well) and to release encapsulated DNA and PNA. PNA and DNA are known to be stable to treatment with ammonia. Because the digested PGLA monomers and cryoprotectant (trehalose) have no significant UV absorbance above 230 nm, the amount of total PNA and DNA in the digest can be determined directly by UV absorbance. Additionally, aliquots of the digest may be analyzed by high performance liquid chromatography (HPLC) and other means (e.g. OliGreen/RiboGreen) to accurately determine the respective amount of PNA and DNA in the initial particle sample.

Additionally, particles may be deformulated by dissolving them in DMSO (e.g., at 65° C. for 5 minutes) followed by dilution with a buffer, e.g., TE buffer at pH 7.4. As outlined above, aliquots of the digest may be analyzed by high performance liquid chromatography (HPLC) and other means (e.g. OliGreen/RiboGreen) to accurately determine the respective amount of PNA and DNA in the initial particle sample.

### Digestion Procedure

1. The nanoparticles (2-4 mg exclusive of weight of excipients such as trehalose) were weighed or dried into a tared 2 mL screw cap polypropylene tube. If the nanoparticles were in an un-tared tube, see Notes 1 & 2, below.

2. 0.5 mL of aqueous ammonia was added to the tube and the screw cap was tightened (28-30% by weight ammonia in water, JT Baker P/N 9721-02).

3. The tube was vortexed occasionally over 10 minutes to suspend nanoparticles. After about 20 minutes the solution was clear with all nanoparticles and solids dissolved.

4. The tube was then left to stand at room temperature for 3 hours.

5. The tube was then spun in a centrifuge to ensure that all liquid was at the tube bottom.

6. The tube was then cooled in dry ice or −20 freezer for 20 minutes.

7. The open tube was then placed in a Speed Vac and concentrated for 1 hour or until volume was 200 μL.

8. About 200 μL of MQ water was then added and the drying in a Speed Vac was repeated.

9. About 200 μL of MQ water was again added and the drying in a Speed Vac was repeated until 200-250 μL of solution remains and the solution had no ammonia smell (See: Note 3, below).

10. The tube was weighed to determine the volume of liquid in the tube (assuming that density of solution is 1 μg/mL).

crude approximation of total nucleic acid (NA—Meaning, in this Case, the Total PNA and DNA) as Weight Percent

1. Using a NanoDrop spectrophotometer or other small volume photometer the spectrum and A260 nm of 1.5 μL of the digestions mix was recorded, against a MQ water blank.

2. If “blank” particles were available from the same formulation run, these were used as the blank to ensure that the PLGA and other materials (other than PNA and DNA) used to produce particles were not confounding or contributing to the absorbance measurements.

3. To estimate the total nucleic acid (i.e. PNA and DNA) an extinction coefficient (EC), which is the average of the EC's for the PNA and DNA, was used. For example, if the PNA EC is 260/pmol and the DNA EC is 600/pmol then the average is ((260+600)/2)=430/pmol. Similarly, an average molecular weight (MW) for the two compounds was used. For example, if the PNA MW is 10,000 and the DNA MW is 20,000, then the average MW is 15,000.

4. Using the absorbance value (A260 nm) obtained for the digested sample, the amount of g of PNA+DNA was calculated and that value was divided by 10 and then divided by the amount of particles digested (mg) to obtain the weight percent (i.e. Xμg/(1000 μg/mg))/Xmg×100=(Xμg/10)/Xmg).

### PNA Measurement as Pmol/Mg Particles or Weight Percent

1. To accurately determine the amount of PNA present in the nanoparticles (see Footnote 4), reversed-phase HPLC was performed using typical conditions (C₁₈ column run at 55° C., Buffer A 0.1% TFA/water, Buffer B 0.1% TFA/ACN, linear gradient).

2. A PNA standard curve was generated by injecting known amounts of the pure PNA on the HPLC. Typically, this was done by performing at least four injections of different amounts covering a range from 5 to 100 pmol of PNA. The area vs. pmol injected was plotted to obtain the standard curve (y=mx+b). The linear regression should have R²>0.97.

3. To determine the amount of PNA in a sample, an amount of the deformulated material (which was 50-75 pmol of total nucleic acid (NA) based upon total NA measurement obtained as described above, was then injected on the HPLC under the same conditions as used to generate the standard curve. For example, if the absorbance measurement for total NA provides a solution which is 1-2 nmol/per mL of digest (using the average EC of 430 OD/gmol) then about 25-50 μL of sample was injected. If the response was too low or not within the standard curve, the amount to be injected was adjusted and reinjected until a response within the standard curve was obtained.

4. The PNA peak area of the sample from the digest was then measured and the standard curve was used to determine the pmol of PNA in the injection volume.

5. The amount of PNA in the total volume of digest was then back calculated and this number was divided by the weight (mg) of particles digested to obtain the pmol/mg value. The PNA MW was also used to convert to weight percent of PNA if needed.

### DNA Measurement as Weight Percent or Pmol/Mg Particles

1. The manufacturer's protocol for the OliGreen or RiboGreen assay using pure oligonucleotide was used to generate a standard curve for measuring the DNA content of the particles. Typical values of the curve will be in μg.

2. Using the total NA estimate (g) from the procedure performed as described above and assuming that 50% of the value is DNA, a series of dilutions on a portion of the digest were performed such that one or more dilutions fell within the range of the standard curve.

3. The diluted samples with OliGreen or RiboGreen were measured and using the standard curve the amount of DNA was determined in the diluted samples.

4. The amount of DNA (g) present in total digest was then back calculated.

5. Total amount DNA for the digested sample (mg) was used to calculate weight percent of DNA (i.e. Xμg/(1000 μg/mg))/Xmg×100=(Xμg/10)/Xmg).

6. If needed, the MW of the DNA was used to convert the value to pmol DNA/mg nanoparticles.

### Footnotes

1. If the particles were in a screw cap tube that had not been tared, then the tube was weighed with particles in it. The digest and assays were then performed and the contents removed from tube. The tube was then dried and weighed to determine mass of particles originally present.

2. If the particles were in a tube that does not have a screw cap (e.g., snap-cap Eppendorf), then the ammonia used for the dilution was used to transfer the particles to another tube that had a screw cap. To do this, 200 μL of ammonia was added to the initial tube, this was mixed until the particles were fully suspended or dissolved. This solution was then transferred to a tared screw cap tube. 3×100 μL portions of ammonia were used to rinse the initial tube, transferring each portion to the screw cap tube. This solution was then incubated for 3 hours as described in Step 4 of the digestion procedure as discussed above.

3. It is important not to dry the digest completely or use heat while drying in the Speed Vac as we have observed that this leads to lower recoveries as compared to partial drying and no heat.

4. Additional data collected indicated that some of the side chain amine groups on lysine moieties of the PNA oligomers were modified with glycolic and lactic acid—presumably during treatment with ammonia. These modifications potentially affect use of this methodology for absolute quantification.

### Example 3: Preparation of Nanoparticles for Gene Editing and Gene Editing Results

In this experiment, 10 mg DNA (SEQ ID No: 2) was dissolved in 80 mL of WFI water, 10 mg PNA (PNA-1) was dissolved in 2 mL of WFI water, a mixture of polymers including PLGA (MW 31,000, 50:50, LA:GA) and mPEG-PLA (MW 2,000:20,000) were dissolved in 38 mL of acetone based on composition in Table 2. PNA was then added into polymer solution to achieve a final concentration of 0.25 mg/mL in 95% acetone.

TABLE 2
Polymer Composition

| | MW (Da) | Mol % | Wt % | Weight (mg) |
|---|---|---|---|---|
| PLGA | 31,000 | 98 | 98.57 | 197.14 |
| mPEG-PLA | 22,000 | 2 | 1.43 | 2.86 |

DNA solution and PNA/polymer solution was then mixed through a “Tee” or “T” mixer at flow rate of 80 mL/min and 40 mL/min, respectively.

The above suspension was diluted with 240 mL of WFI water through another “Tee” (“T”) mixer.

Trehalose was added to the above solution to achieve a final concentration of 8% (wt/wt).

The suspension was then diafiltrated against 2.88 liter of 8% trehalose through a 100 kD MWCO membrane on TFF and then was further concentrated to 5 mL.

Particle size and PDI was measured using Malvern zetasizer, DNA and PNA concentration was determined on Nanodrop Spectrometer. Formulation was further diluted to a final API concentration of 1 mg/mL and then stored at −20° C. freezer. Data for the resulting nanoparticles can be found in FIGS. 1a to 14c.

Total bone marrow was harvested from the femurs and tibias of sickle cell Townes mice. Cells were filtered through a 30 micron mesh into a single cell suspension and cultured in 24-well plates in Roswell Park Memorial Institute (RPMI) culture medium supplemented with 20% fetal bovine serum (FBS). Nanoparticles formulated with PNA (PNA-1) and donor DNA (SEQ ID NO: 2) as APIs as described above (final 0.1 mg/mL total API) was added to cells. Cells treated with empty nanoparticles (i.e. no API) were included as negative control. After 48 hours of incubation, cells were harvested, washed with phosphate-buffered saline (PBS) and subjected to whole genomic DNA extraction using Promega Wizard SV Genomic DNA purification kit. Double-stranded DNA concentration was measured by fluorometrically by Qubit Fluorometer with double stranded DNA (dsDNA) High Sensitivity (HS) Assay Kit before using digital droplet PCR (ddPCR) to evaluate the percentage of gene editing (FIG. 11). Primer sequences are as follows: primer-forward (5′-CACCAACTTCATCCACGTTCAC-3′ (SEQ ID NO: 5)); primer-reverse (5′-TCTATTGCTTACATTTGCTTCTGACA-3′ (SEQ ID NO: 6). Probes are designed with 5′ Dye and 3′ minor groove binder non-fluorescent quencher (MGBNFQ): mutant (VIC®), (5′-CAGACTTCTCCACAGGA-3′ (SEQ ID NO: 10)); wildtype (fluorescein amidite; FAM) (5′-CAGACTTCTCCTCAGGA-3′ (SEQ ID NO: 11)). PCR was performed under the following conditions: 95° C., 10 min; ×40 [94° C., 30 s; 54.8° C., 4 min ramp 2° C./s]; 98° C., 10 min; 4° C. forever. As shown in FIGS. 1a to 14c, nanoparticles prepared by the processes disclosed herein demonstrated gene correction activity in sickle cell disease mouse bone marrow cells with a gene editing activity of 1-3%.

SC1 human cell line (homozygous for sickle mutation) were cultured at density of 0.5 million cells in final volume of 0.5 mL complete media (RPMI plus 20% FBS). For dose-dependent study, cells were treated with increasing doses of nanoparticles for 48 hours. Untreated cells were included as negative control. Cells were harvested, washed and then lysed to isolate whole genomic DNA. Samples were evaluated fluorometrically by Qubit Fluorometer with double stranded DNA (dsDNA) High Sensitivity (HS) Assay Kit and later analyzed by ddPCR using the condition described above with the addition of single stranded DNA (5′-CTTCTCCACAGGAGTCAGGTGC/3Phos-3′; SEQ ID NO: 7) to reduce the interference of PNA (PNA-1) that might be present in the sample during amplification. A dose-dependent (0-0.4 mg/mL) increase in gene editing was observed (FIG. 12a). At 0.4 mg API/mL, a 6% gene correction was observed.

To evaluate the effect of exposure length on gene editing, cells harvested and prepared as described above were treated with 0.1 mg/mL of nanoparticles comprising PNA (PNA-1) and DNA (SEQ ID NO: 2) and harvested after 24, 48, 72, and 96 hours. After washing, genomic DNA was isolated from the cells and evaluated using Qubit. Editing as measured by ddPCR increased by time, plateauing around 48 to 72 hours post treatment (FIG. 12b).

To measure the effect of repeated treatment on gene editing, cells harvested and prepared as described above were first incubated with 0.1 mg/mL nanoparticles comprising PNA (PNA-1) and DNA (SEQ ID NO: 2) for 48 hours. Cells were then washed and resuspended in fresh media and treated with fresh supply of 0.1 mg/mL nanoparticles comprising PNA (PNA-1) and DNA (SEQ ID NO: 2) for additional 48 hours. Genomic DNA from these samples was prepared and evaluated by ddPCR to measure percentage of gene editing. As shown in FIG. 12c, repeated treatment of cells in vitro increased the correction rate of SCD mutation from 2.5% to 6% (FIG. 12c).

Gene editing was measured in peripheral blood mononuclear cells (PBMCs) from 4 individual sickle cell anemia patients. Patient PBMCs were resuspended in complete media (RPMI with 20% FBS) at density of 0.2×10⁶ cells/mL and were treated with 0.1 mg/mL nanoparticles comprising PNA (PNA-1) and DNA (SEQ ID NO: 2) for 48 hours. After washing with PBS, whole genomic DNA was isolated, and samples were subjected to ddPCR. Percent editing achieved in primary patient cells in vitro varied among different patient samples (4 to 14%, FIG. 13a) and was generally higher than editing often achieved in the cell line obtained from Townes mice under similar conditions.

PBMCs from a primary sickle cell anemia patient sample was used to isolate CD34+ hematopoietic stem and progenitor cell (HSPC) population using Miltenyi CD34 positive selection magnetic beads (following manufacturer's instructions). CD34+ HSPCs and remaining PBMCs depleted of CD34+ cells were cultured side-by-side in StemSpan SFEM II media with CD34+ Expansion Supplement (contains recombinant human FMS-like tyrosine kinase 3 ligand (FIt3L), stem cell factor (SCF), interleukin-3 (IL-3), interleukin-6 (IL-6), and thrombopoietin (TPO)). Later, nanoparticles comprising PNA (PNA-1) and DNA (SEQ ID NO: 2) were added to cells at final 0.1 mg/mL API and incubated for 48 hours. Untreated samples were included as negative controls. Cells were collected and washed for genomic DNA extraction and gene editing was measured using ddPCR (FIG. 13b). With reference to FIG. 13b, gene editing in CD34+ hematopoietic stem cells was slightly less than 3 percent as compared with PBMCs with C₃₄+ cells removed, wherein the editing appears to be slightly higher.

The correction of sickle cell mutation was further confirmed by next generation sequencing (NGS). Amplicons were prepared using PCR and primers designed around SCD mutation in human hemoglobin gene (Forward: 5′-TTGTAACCTTGATACCAACC-3′ (SEQ ID NO: 8) and Reverse: 5′-CTTACATTTGCTTCTGACAC-3′ (SEQ ID NO: 9), PCR conditions: 95° C., 3 min; ×35 [95° C., 30 s; 49.6° C., 30 s; 72° C., 1 min]; 72° C., 10 min; 4° C. forever). PCR products were subjected to column clean-up (QIAquick Qiagen) and amplicon were evaluated on Qubit and later on 2% gel for size and purity. NGS analysis of the samples was performed by a fee for service provider on a blind basis using the IIIumina TruSeq Paired-End Sequencing workflow. Samples were then sequenced on IIIumina MiSeq (2×150 bp) platform (merged paired reads). Unique nucleotide sequences in the region of interest were identified and a relative abundance was calculated for each unique sequence. In edited samples, variant abundance of unique sequences with correction of mutant A to wild-type T is calculated and shown in FIGS. 14a to 14c (next to percent editing measured using ddPCR on genomic DNA of these samples). These data presented in FIG. 14a to 14c demonstrate that gene editing observed in the ddPCR was generally confirmed by the NGS results.

### Example 4: Preparation of Nanoparticles for Gene Editing and Gene Editing Results

In this experiment, 3.72 mg DNA (SEQ ID No: 2) was dissolved in 30 mL of WFI water. 3.72 mg PNA (PNA-1) was dissolved in 0.75 mL of WFI water and added to a mixture of polymers including PLGA (MW 31,000, 50:50, LA:GA) and mPEG-PLA (MW 2,000:20,000) which were dissolved in 14.25 mL of acetone. The final PNA concentration was 0.25 mg/mL in 95% acetone (95/5 acetone/water (v/v) based on composition in Table 3.

TABLE 3
Polymer Composition

| | MW (Da) | Mol % | Wt % | Weight (mg) |
|---|---|---|---|---|
| PLGA | 31,000 | 98 | 98.9 | 71.3 |
| mPEG-PLA | 22,000 | 2 | 1.1 | 0.78 |

DNA solution and PNA/polymer solution was then mixed through a “Tee” or “T” mixer at flow rate of 10 mL/min and 5 mL/min, respectively.

The above suspension was diluted with 90 mL of WFI water through another “Tee” (“T”) mixer.

Trehalose was added to the above solution to achieve a final concentration of 8% (wt/wt).

The suspension was then diafiltrated against 1.08 liter of 8% trehalose through a 100 kD MWCO membrane on TFF and then was further concentrated to 5 mL.

Particle size and PDI was measured using Malvern zetasizer. DNA and PNA concentration was determined on Nanodrop Spectrometer. Formulation was flash frozen and then stored at −20° C. freezer. Data for the resulting nanoparticles can be found in Table 4.

TABLE 4

| | |
|---|---|
| Formulation | B26066 |
| Organic Solvent | Acetone |
| Size (nm) | 153.9 |
| PDI | 0.36 |
| [DNA] (ug/mL) | 300 |
| [PNA] (ug/mL) | 240 |
| PNA yield (%) | 30.1 |
| DNA yield (%) | 37.6 |
| DNA/PNA ratio | 1.25 |
| Method | T-Mix |
| Purification | TFF 100 kD MWCO |

SC1 human cell line (homozygous for sickle mutation) were cultured at density of 0.5 million cells in final volume of 0.5 mL complete media (RPMI plus 20% FBS). For dose-dependent study, cells were treated with increasing doses of freshly prepared nanoparticles for 48 hours. Untreated cells were included as negative control. Cells were harvested, washed and then lysed to isolate whole genomic DNA. Samples were evaluated fluorometrically by Qubit Fluorometer with double stranded DNA (dsDNA) High Sensitivity (HS) Assay Kit and later analyzed by ddPCR using the condition described above with the addition of single stranded DNA (5′-CTTCTCCACAGGAGTCAGGTGC/3Phos-3′; SEQ ID NO: 7) to reduce the interference of PNA (PNA-1) that might be present in the sample during amplification. A dose-dependent increase in gene editing was observed.

With reference to FIG. 15A, data is presented for SC1 cells treated with the nanoparticle formulation prepared as described above. At 0.03 mg/mL DNA and 0.024 mg/mL PNA, gene editing at an average of about 2% was observed. At 0.1 mg/mL DNA and 0.08 mg/mL PNA, gene editing of about 5% was observed.

After treating cells as described above, the remaining formulation (i.e. formulation B26066), was stored frozen for 18 days and then the formulation was allowed to thaw. The formulation was then retested for gene editing activity in SC-1 cells (now about 3 weeks after production date) as described above. The gene editing data is shown below in FIG. 15B.

With reference to FIGS. 15A and 15B, it is clear that some gene editing was observed with the stored formulation at the highest dose (FIG. 15B), but far less editing than was observed with the freshly made formulation at that dose (compare FIG. 15A with FIG. 15B when dosing at 0.1 mg/mL DNA and 0.08 mg/mL PNA).

### Example 5: Effect of Storage Condition on Nanoparticles for Gene Editing

PLGA nanoparticles were prepared as described in Example 4, above, except that were concentrated on TFF approximately 2-fold less than B26066.

After TFF, the lot of nanoparticles was split into 3 aliquots; (i) one stored as a suspension at 4° C.; (ii) one flash frozen and stored at −20° C.; and (3) one flash frozen and lyophilized. The composition and characterization of the nanoparticles as formulated (prior to preparation for storage) is presented in Table 5. The lyophilized nanoparticles were reconstituted in 500 μL of water which provided for approximately equal PLGA/API concentration in all test articles.

TABLE 5

| | |
|---|---|
| Formulation | B26086 |
| Organic Solvent | Acetone |
| Size (nm) | 102.4 |
| PDI | 0.235 |
| [DNA] (ug/mL) | 144.6 |
| [PNA] (ug/mL) | 132.9 |
| PNA yield (%) | 16.7 |
| DNA yield (%) | 18.1 |
| DNA/PNA ratio | 1.09 |
| Method | T-Mix |
| Purification | TFF 100 kD MWCO |

After approximately 24 hours of storage, the nanoparticles for each of the three storage conditions were tested on SC1 cells and analyzed for gene editing as described in Example 4, above. The gene editing data for nanoparticles stored under these various conditions is presented in FIG. 16.

With reference to FIG. 16, the data appears to demonstrate that this batch of nanoparticles edited in all cases (approximately between 1 and 2% editing) without regard to storage conditions, but that the sample subjected to freezing for 24 hours performed best.

### Example 6: Preparation of Nanoparticles Containing PNA

In this experiment, 0.05 mL of 5 mg/mL PNA in water (SEQ ID NO: 2) was added to 0.95 mL of 5 mg/mL PLGA and 0.01 mL of mPEG-PLGA 5 mg/mL (MW 2,000:20,000) which were dissolved in acetone. The final PNA concentration was 0.25 mg/mL in 95% acetone.

DNA solution and PNA/polymer solution was then mixed through a microfluidic chip (NanoAssemblr, Precision Nanosystems) at a rate of 15 mL/min with 2 parts aqueous mixed with 1 part organic.

The resultant particle size was measured by dynamic light scattering and sample was filtered through a 0.45 um PES syringe filter. The suspension (3 mL) was passed down a Sepharose CL-2B size exclusion chromatography column pre-equilibrated with 8% trehalose. The purified suspension was collected in the void volume, flash frozen and lyophilized. After reconstitution in water, particle size was determined by light scattering and the PNA measured as described previously. The results are shown below in Table 6.

TABLE 6

| | | | | | | Lyophilized/ | | |
|---|---|---|---|---|---|---|---|---|
| | | | PLGA | | | reconstituted | | |
| | | % | Mol. Wt | diameter | | diameter | PDI | PNA |
| PLGA | Supplier | Lactide:glycolide | (kDa) | (nm) | PDI | (nm) | Lyo/recon | (ug/mL) |
| B6010-2 | Durect | 50:50 | 40,000 | 262.4 | 0.107 | 369.7 | 0.241 | 1.11 |
| AP040 | PolySci | 50:50 | 15,000-25,000 | 97 | 0.041 | 251.6 | 0.111 | 1.77 |
| | Tech | | | | | | | |
| AP061 | PolySci | 75:25 | 35,000-45,000 | 321.8 | 0.059 | 456 | 0.205 | 0.80 |
| | Tech | | | | | | | |
| AP132 | PolySci | 75:25 | 25,000-35,000 | 95.15 | 0.059 | 232.5 | 0.207 | 2.60 |
| | Tech | | | | | | | |

While the present teachings are described in conjunction with various embodiments, it is not intended that the present teachings be limited to such embodiments. On the contrary, the present teachings encompass various alternatives, modifications and equivalents, as will be appreciated by those of skill in the art.

1. A method comprising:
a) combining a first solution and a second solution at a junction under conditions suitable to produce nanoprecipitation in a post-junction fluid stream comprising post-junction fluid,
wherein:
(i) the first solution comprises a first solvent; and
(ii) the second solution comprises: (w) a neutral or positively charged nucleic acid mimic (NPNAM); (x) a polymer, and (y) a second solvent;
b) forming a nanoparticle comprising the polymer and the NPNAM in the post-junction fluid stream.

2. The method of claim 1, wherein the polymer comprises a synthetic polymer.

3. The method of claim 1, wherein:
(i) the first solution comprises water or an aqueous solution or buffer; and
(ii) the second solution comprises: (w) a neutral or positively charged nucleic acid mimic (NPNAM); (x) a polymer and (y) a water miscible organic solvent.

4. The method of claim 1, wherein said second solution comprises a second NPNAM or a second polymer.

5. (canceled)

6. The method of claim 1, wherein the NPNAM is encapsulated and/or entrapped in the nanoparticle.

7-8. (canceled)

9. The method of claim 1, wherein the first solution further comprises a load component.

10-12. (canceled)

13. The method of claim 9, wherein the load component comprises a nucleic acid that:
a) is a DNA oligomer of between 20 and 100 nucleotides in length;
b) comprises one or more phosphorothioate linkages; and/or
c) comprises at least two phosphorothioate linkages at each of its 3′ and 5′ termini.

14-18. (canceled)

19. The method of claim 1, wherein the NPNAM comprises a structure of Formula (I): 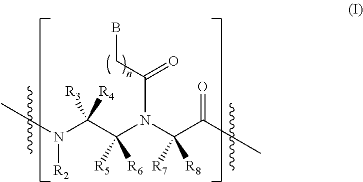
wherein B is a nucleobase;
R₂ is hydrogen, deuterium, or C₁-C₄ alkyl;
each of R₃, R₄, R₅, R₆, R₇ and R₈ is independently selected from the group consisting of: hydrogen, deuterium, fluorine, and a side chain selected from the group consisting of: IIIa, IIIb, IIIc, IIId, IIIe, IIIf, IIIg, IIIh, IIIi, IIIj, IIIk, IIIm, IIIn, IIIo, IIIp, IIIq, IIIr, IIIs, IIIt, IIIu, IIIv, IIIw, IIIx, IIIy, IIIz, IIIaa and IIIab, wherein each of IIIi, IIIj, IIIk, IIIm, IIIn, IIIo, IIIp, IIIq, IIIr, IIIs, IIIt, IIIu, IIIv, IIIw, IIIx, IIIy, and IIIz independently and optionally comprise a protecting group; 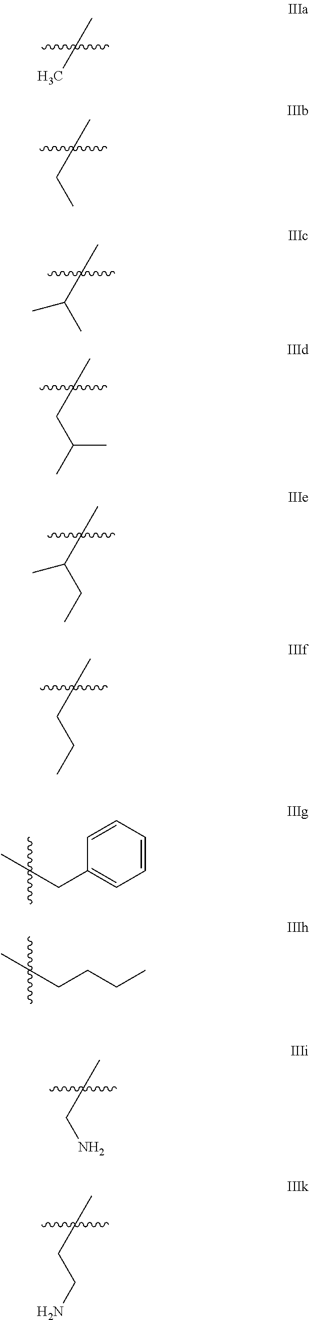 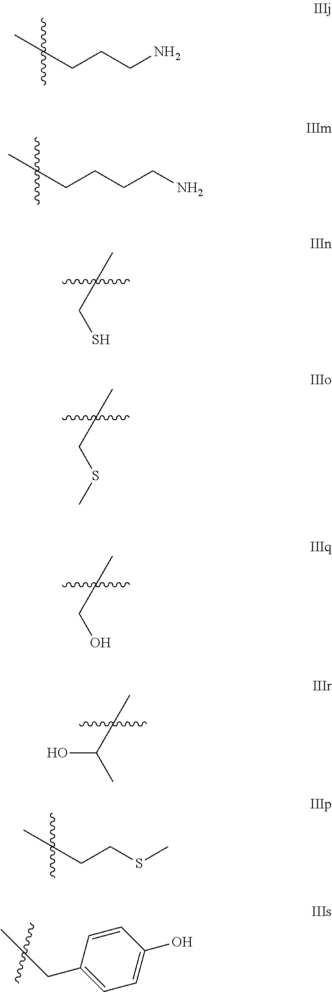
R₁₆ is selected from H, D and C₁-C₄ alkyl group; n is an integer from 0 to 3, inclusive; p is an integer from 0 to 10, inclusive; and
the symbol “ [symbol] ” cutting across a bond indicates a point of attachment of the moiety illustrated to another atom, moiety or chemical structure (or subcomponent thereof).

20-35. (canceled)

36. The method of claim 1, wherein the polymer is selected from polymers or polymer conjugates, co-polymers, block polymers, polymer mixtures and/or polymer blends of: polylactic acid (PLA), polyglycolic acid, (PGA), poly(lactic-coglycolic acid) (PLGA), poly(4-hydroxy-L-proline ester), other degradable polyesters, polyanhydrides, poly(ortho)esters, polyesters, polyurethanes, poly(butic acid), poly(valeric acid), poly(caprolactone), poly(hydroxyalkanoates), poly(lactide-co-caprolactone), poly(amine-co-ester) polymers and polyethylene glycol (PEG) polymers, and a combination of any two or more of the foregoing.

37-42. (canceled)

43. The method of claim 1, wherein the method further comprises the step of: (c) adding a first component or a second component to the post-junction fluid stream.

44-48. (canceled)

49. The method of claim 1, wherein the method further comprises the step of:
(c) diluting the post-junction fluid stream;
(d) adding a surface stabilizer to stabilize the nanoparticles formed in the post-junction fluid stream;
(e) separating or removing NPNAM that is not associated with a nanoparticle from the post-junction fluid stream;
(f) removing the organic solvent from the post-junction fluid stream;
(g) sterilizing the post-junction fluid comprising the nanoparticles; and/or
(h) finish and filling a sterile container with the nanoparticles.

50-65. (canceled)

66. A nanoparticle comprising:
a) a synthetic polymer; and
b) a neutral or positively charged nucleic acid mimic (NPNAM(s));
wherein the nanoparticle comprises one of the following properties:
(i) the amount of neutral or positively charged nucleic acid mimic(s) encapsulated and/or entrapped within the nanoparticle is greater than or equal to 2 percent (2%) by weight of NPNAM(s) to the total weight of the nanoparticle(s);
(ii) the diameter of the nanoparticle is between about 30 to about 350 nanometers; or
(iii) the neutral to negative surface charge of the nanoparticle is less than about −100 my.

67-75. (canceled)

76. The nanoparticle of claim 66, wherein the NPNAM comprises a structure of Formula (I): 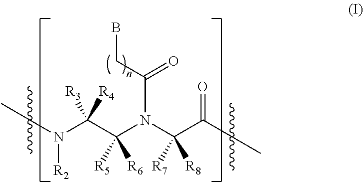
wherein B is a nucleobase;
R₂ can be hydrogen, deuterium or C₁-C₄ alkyl;
each of R₃, R₄, R₅, R₆, R₇ and R₈ can be independently selected from the group consisting of: hydrogen, deuterium, fluorine, and a side chain selected from the group consisting of: IIIa, IIIb, IIIc, IIId, IIIe, IIIf, IIIg, IIIh, IIIi, IIIj, IIIk, IIIm, IIIn, IIIo, IIIp, IIIq, IIIr, IIIs, IIIt, IIIu, IIIv, IIIw, IIIx, IIIy, IIIz, IIIaa and IIIab, wherein each of IIIi, IIIj, IIIk, IIIm, IIIn, IIIo, IIIp, IIIq, IIIr, IIIs, IIIt, IIIu, IIIv, IIIw, IIIx, IIIy, and IIIz independently and optionally comprise a protecting group; 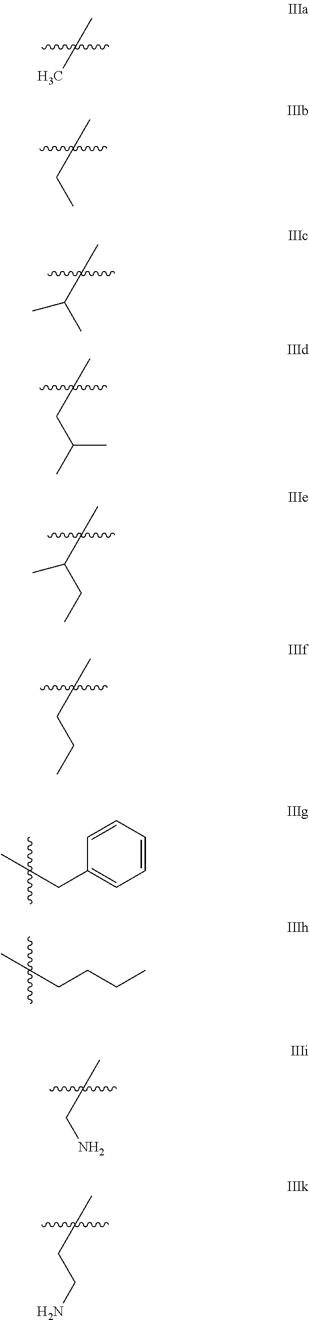 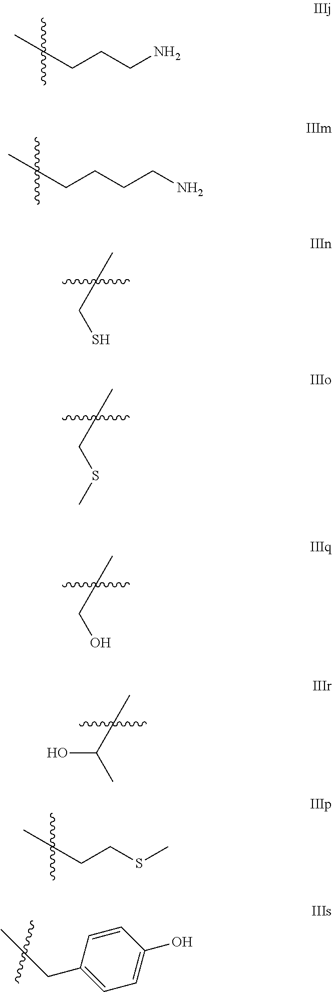
R₁₆ is selected from H, D and C₁-C₄ alkyl group; n is an integer from 0 to 3, inclusive; p is an integer from 0 to 10, inclusive; and
the symbol “ [symbol] ” cutting across a bond indicates a point of attachment of the moiety illustrated to another atom, moiety or chemical structure (or subcomponent thereof).

77-82. (canceled)

83. The nanoparticle of claim 66, wherein the nanoparticle further comprises a load component.

84-92. (canceled)

93. A preparation comprising a plurality of nanoparticles, wherein each nanoparticle of the plurality comprises:
a) a synthetic polymer; and
b) a neutral or positively charged nucleic acid mimic (NPNAM(s));
wherein the preparation comprises one of the following properties:
(i) the amount of neutral or positively charged nucleic acid mimic(s) encapsulated and/or entrapped within each nanoparticle of the plurality is greater than or equal to 0.05% by weight of NPNAM(s) to the total weight of the nanoparticle(s);
(ii) at least 5% of the nanoparticles of the preparation have an average diameter of between 5 and 500 nm;
(iii) at least 5% of the nanoparticles of the preparation have a neutral to negative surface charge of less than −100 my;
(iv) the preparation contains less than 0.05% by weight of free NPNAM, free synthetic polymer, or a free load component; and
(v) the preparation contains less than 0.05% by weight of empty nanoparticles.

94-105. (canceled)

106. The preparation of claim 93, wherein the NPNAM comprises a structure of Formula (I): 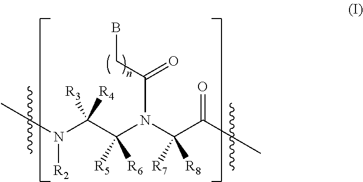
wherein B is a nucleobase;
R₂ can be hydrogen, deuterium or C₁-C₄ alkyl;
each of R₃, R₄, R₅, R₆, R₇ and R₈ can be independently selected from the group consisting of: hydrogen, deuterium, fluorine, and a side chain selected from the group consisting of: IIIa, IIIb, IIIc, IIId, IIIe, IIIf, IIIg, IIIh, IIIi, IIIj, IIIk, IIIm, IIIn, IIIo, IIIp, IIIq, IIIr, IIIs, IIIt, IIIu, IIIv, IIIw, IIIx, IIIy, IIIz, IIIaa and IIIab, wherein each of IIIi, IIIj, IIIk, IIIm, IIIn, IIIo, IIIp, IIIq, IIIr, IIIs, IIIt, IIIu, IIIv, IIIw, IIIx, IIIy, and IIIz independently and optionally comprise a protecting group; 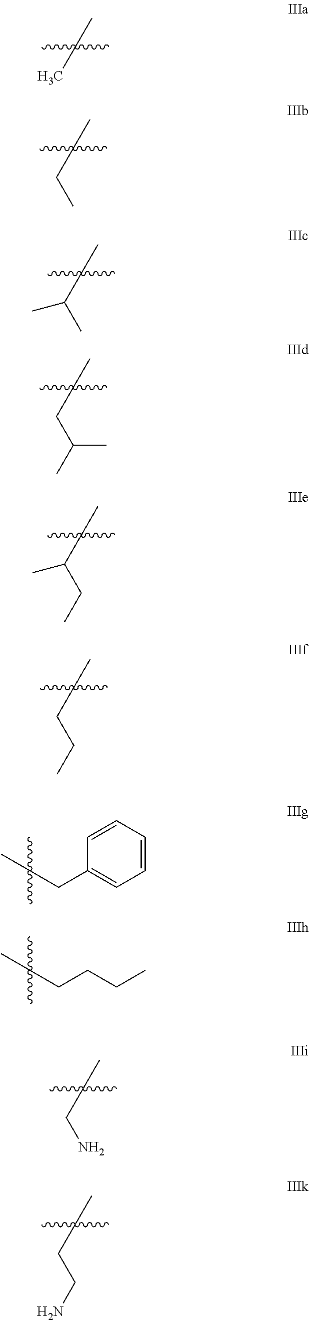 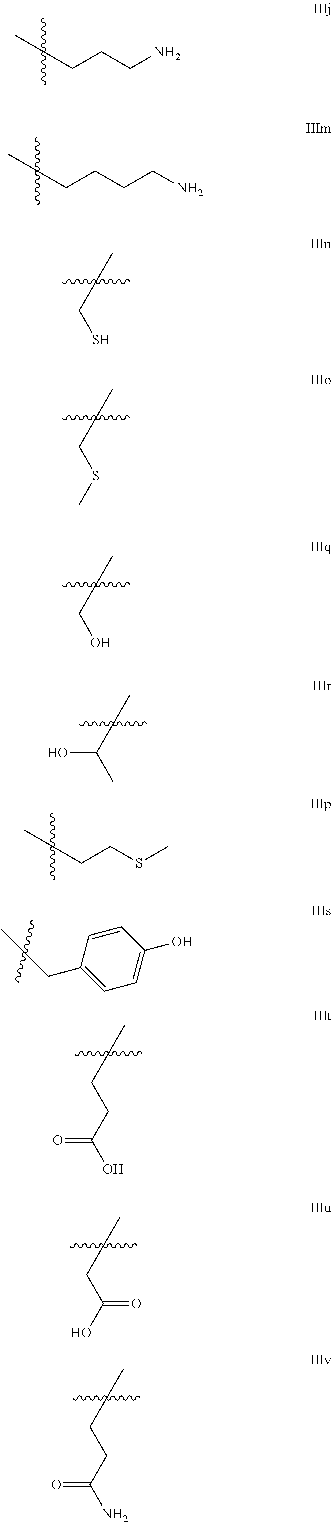 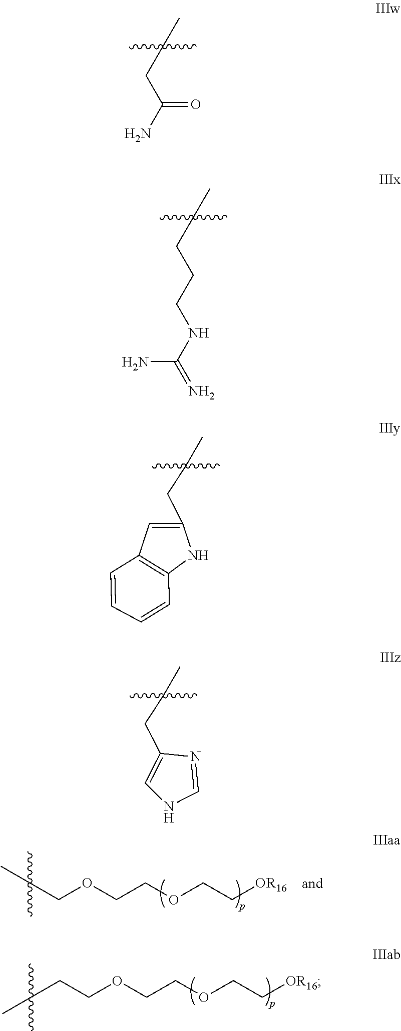
R₁₆ is selected from H, D and C₁-C₄ alkyl group; n is an integer from 0 to 3, inclusive; p is an integer from 0 to 10, inclusive; and
the symbol “ [symbol] ” cutting across a bond indicates a point of attachment of the moiety illustrated to another atom, moiety or chemical structure (or subcomponent thereof).

107-124. (canceled)

125. An apparatus comprising:
a) a first solvent supply comprising at least water or an aqueous buffer;
b) a second solvent supply comprising at least one water miscible organic solvent and at least one neutral or positively charged nucleic acid mimic;
c) a junction to which the first solvent supply and second solvent supply are in fluid connection; and
d) a post-junction chamber suitable to contain a post-junction fluid stream; and
wherein said junction permits mixing of solvent forced through said junction simultaneously from the first and second solvent supplies and into the post-junction conduit.

126-137. (canceled)

138. A method of manufacturing, or evaluating, a nanoparticle or preparation comprising a plurality of nanoparticles comprising:
a) providing a nanoparticle or preparation comprising a plurality of nanoparticles, wherein the nanoparticle comprises a synthetic polymer and an encapsulated and/or entrapped NPNAM, and optionally a load component; and
b) acquiring, directly or indirectly, a value for a preparation parameter; thereby manufacturing, or evaluating, the nanoparticle or preparation comprising the plurality of nanoparticles.

139-146. (canceled)

147. A method of altering a target nucleic acid, comprising:
a) providing a nanoparticle or preparation comprising a plurality of nanoparticles, wherein the nanoparticle comprises a synthetic polymer and an encapsulated and/or entrapped NPNAM, and optionally a load component; and
b) contacting the NPNAM of a nanoparticle with a target nucleic acid under conditions sufficient to alter the target nucleic acid,
thereby altering a target nucleic acid.

148-151. (canceled)

152. The method of claim 147, wherein altering comprises:
i) altering the state of association of the two strands of a target double stranded nucleic acid;
ii) altering the helical structure of a target double stranded nucleic acid;
iii) altering the topology in a strand of target double stranded nucleic acid;
iv) recruiting a nucleic acid modifying enzyme to a target nucleic acid;
v) cleaving a strand of a target double stranded nucleic acid;
vi) altering the sequence of a target nucleic acid; and/or
vii) altering the sequence of a target nucleic acid to the sequence of a template nucleic acid.

153-165. (canceled)
